(19) United States
(12) Patent Application Publication
(10) Pub. No.: US 2024/0391978 A1
(43) Pub. Date: Nov. 28, 2024

(54) APOLIPOPROTEIN FUSION PROTEINS FOR CELL-SPECIFIC IMMUNE REGULATION

(71) Applicant: BIO-TRIP B.V., Eindhoven (NL)

(72) Inventors: Roy VAN DER MEEL, Eindhoven (NL); Willem J.M. MULDER, Eindhoven (NL); Maarten MERKX, Eindhoven (NL); David Pepijn SCHRIJVER, Amsterdam (NL); Anne DE DREU, Helmond (NL); Ayla Martine HOKKE, Waalre (NL); Koen DE BRUIN, Utrecht (NL)

(21) Appl. No.: 18/694,460

(22) PCT Filed: Sep. 23, 2022
(86) PCT No.: PCT/EP2022/076593

§ 371 Date: Mar. 22, 2024

(30) Foreign Application Priority Data
Sep. 23, 2021 (EP) 21198622.9

(51) Int. Cl. C07K 14/775; A61K 9/51; A61K 38/00; A61P 37/04; C07K 14/54; C07K 14/55; C07K 14/705

(52) U.S. Cl. CPC C07K 14/775; A61K 9/5123; A61P 37/04; C07K 14/5406; C07K 14/55; C07K 14/70596; A61K 38/00; C07K 2319/00

(57) ABSTRACT

The invention relates to fusion proteins of apolipoprotein with an immunomodulatory biomolecule and/or a rerouting molecule. The fusion proteins can be used as a carrier for an immunomodulatory biomolecule as such or incorporated in a lipid nanoparticle. The fusion protein finds use in the treatment of immune related disorders, or targeting a payload to a specific target site.

### FIELD OF THE INVENTION

The invention relates to the field of fusion proteins and more particularly fusion proteins that find use in the treatment of immune related disorders. The invention further relates to lipid nanoparticles comprising the fusion proteins and methods of making such. Lastly the invention relates to methods of treatment using the fusion proteins or lipid nanoparticles.

### BACKGROUND OF THE INVENTION

Many promising therapeutics are hampered by poor circulation time due to rapid clearance of the therapeutic from the body. For example, it was found that cytokines may hold very promising uses in many immunological applications. However due to the very short half-life in the body, either no effect can be exerted on the intended target, or toxic amounts are needed to achieve an effect. Therefore, there is a need for improved methods to safely reduce the circulation half-life of therapeutics.

Additionally, many promising therapeutics suffer from the fact that they either do not, or poorly, reach the intended target site, or present undesired off-target effects. Therefore, there is a further need to improve targeting of therapeutics.

These, among other, problems are addressed by the products and methods as defined in the appended claims.

### SUMMARY OF THE INVENTION

The present invention is based on the inventors' finding that apolipoprotein can be used as a carrier for therapeutic agents, and that apolipoproteins may further be modified to target specific cells, tissues or organs. The inventors found that fusion proteins of immunomodulatory biomolecules, such as cytokines, with apolipoproteins or apolipoprotein mimetics demonstrate strongly increased half-life in blood, thereby opening up the possibility to use immunomodulatory biomolecules, such as cytokines, in a therapeutical manner without requiring toxic concentrations to be administered. Further it was realized that apolipoproteins or mimetics thereof allow targeting of immunomodulatory biomolecules, such as cytokines, when fused together.

It was further realized that it is possible to direct a fusion protein or apolipoprotein (or mimetic thereof) to an intended target by linking it to a rerouting molecule. Moreover, present inventors unexpectedly found that the fusion of an immunomodulatory biomolecule and/or a rerouting molecule to an apolipoprotein or an apolipoprotein mimetic allows to easily incorporate said immunomodulatory biomolecule and/or a rerouting molecule in a lipid nanoparticle and to expose said immunomodulatory biomolecule and/or a rerouting molecule to the environment surrounding said lipid nanoparticle. In this way the apolipoprotein (or mimetic thereof) or fusion protein can be targeted to cells, tissues or organs it would otherwise not or insufficiently reach, or it could be used to reduce off-target effects.

The fusion proteins may be used as such, meaning not as part of a lipoprotein or lipid nanoparticle. In such way the fusion protein may serve as a carrier to deliver an immunomodulatory biomolecule to a target site. Alternatively, the fusion protein may be used to prepare a lipid nanoparticle.

A first aspect of the invention provides an apolipoprotein lipid nanoparticle comprising
a fusion protein comprising an apolipoprotein or an apolipoprotein mimetic and an immunomodulatory biomolecule; and
phospholipids;
wherein the immunomodulatory biomolecule is a protein that enhances or suppresses an immune response.

A further aspect of the invention provides an apolipoprotein lipid nanoparticle comprising
a fusion protein comprising an apolipoprotein or an apolipoprotein mimetic and a rerouting molecule; and
phospholipids;
wherein the rerouting molecule is a molecule that allows that fusion protein to bind to a different target than to which the apolipoprotein or apolipoprotein mimetic would have bound and/or to bind to its intended target with a higher affinity.

A further aspect of the invention provides an apolipoprotein lipid nanoparticle comprising
a fusion protein comprising an apolipoprotein or an apolipoprotein mimetic, an immunomodulatory biomolecule, and a rerouting molecule; and
phospholipids;
   wherein the immunomodulatory biomolecule is a protein that enhances or suppresses an immune response; and
   wherein the rerouting molecule is a molecule that allows that fusion protein to bind to a different target than to which the apolipoprotein or apolipoprotein mimetic would have bound and/or to bind to its intended target with a higher affinity.

A further aspect of the invention provides an apolipoprotein lipid nanoparticle comprising
a fusion protein comprising an apolipoprotein or an apolipoprotein mimetic and an immunomodulatory biomolecule; wherein the immunomodulatory biomolecule is a protein that enhances or suppresses an immune response;
a fusion protein comprising an apolipoprotein or an apolipoprotein mimetic and a rerouting molecule; wherein the rerouting molecule is a molecule that allows that fusion protein to bind to a different target than to which the apolipoprotein or apolipoprotein mimetic would have bound and/or to bind to its intended target with a higher affinity; and
phospholipids.

A further aspect of the invention provides a method of manufacturing an apolipoprotein lipid nanoparticle as described herein, the method comprising the steps of:
a1) expressing and isolating one or more apolipoprotein fusion proteins to obtain one or more isolated apolipoprotein fusion proteins,
wherein the one or more apolipoprotein fusion proteins are selected from the group consisting of: an apolipoprotein or apolipoprotein mimetic fused to an immunomodulatory biomolecule;
an apolipoprotein or apolipoprotein mimetic fused to a rerouting molecule;
an apolipoprotein or apolipoprotein mimetic fused to an immunomodulatory biomolecule and a rerouting molecule; and combinations thereof; and/or
a2) chemically conjugating one or more apolipoproteins or apolipoprotein mimetics and isolating the one or more conjugated apolipoproteins to obtain one or more isolated conjugated apolipoproteins,
   wherein the one or more conjugated apolipoproteins are selected from the group consisting of: an apolipoprotein or apolipoprotein mimetic conjugated to an immunomodulatory biomolecule;
an apolipoprotein or apolipoprotein mimetic conjugated to a rerouting molecule;
an apolipoprotein or apolipoprotein mimetic conjugated to an immunomodulatory biomolecule and a rerouting molecule; and combinations thereof; and
b) combining the one or more isolated apolipoprotein fusion proteins obtained in step a1 and/or the one or more isolated conjugated apolipoproteins obtained in step a2 with phospholipids, and optionally sterols and/or lipids, to obtain an apolipoprotein lipid nanoparticle.

A further aspect of the invention provides an apolipoprotein lipid nanoparticle obtained by or obtainable by the method as taught herein.

A further aspect of the invention provides a pharmaceutical composition comprising the apolipoprotein lipid nanoparticle as taught herein, and a pharmaceutically acceptable carrier.

A further aspect of the invention provides the apolipoprotein lipid nanoparticle as taught herein or the pharmaceutical composition as taught herein for use as a medicament.

A further aspect of the invention provides the apolipoprotein lipid nanoparticle as taught herein or the pharmaceutical composition as taught herein for use in the treatment of an immune related disorder.

A further aspect of the invention provides the apolipoprotein lipid nanoparticle as taught herein or the pharmaceutical composition as taught herein for use in targeting said immunomodulatory biomolecule to a target cell, preferably a myeloid cell.

A further aspect of the invention provides the use of the apolipoprotein lipid nanoparticle as taught herein for delivering an immunomodulatory biomolecule to a target, preferably wherein the target is a cell, tissue, and/or organ.

A further aspect of the invention provides a fusion protein comprising an apolipoprotein or an apolipoprotein mimetic and an immunomodulatory biomolecule, wherein the immunomodulatory biomolecule is a protein that enhances or suppresses an immune response, for use in targeting said immunomodulatory biomolecule to a myeloid cell.

A further aspect of the invention provides a fusion protein comprising an apolipoprotein or an apolipoprotein mimetic and a rerouting molecule, wherein the rerouting molecule is a molecule that allows that fusion protein to bind to a different target than to which the apolipoprotein or apolipoprotein mimetic would have bound and/or to bind to its intended target with a higher affinity.

A further aspect of the invention provides a nucleic acid encoding the fusion protein comprising the apolipoprotein or an apolipoprotein mimetic and the rerouting molecule as taught herein.

A further aspect of the invention provides a pharmaceutical composition comprising the fusion protein as taught herein or the nucleic acid as taught herein, and a pharmaceutically acceptable carrier.

A further aspect of the invention provides the fusion protein as taught herein, the nucleic acid as taught herein or the pharmaceutical composition as taught herein for use as a medicament.

A further aspect of the invention provides the fusion protein as taught herein, the nucleic acid as taught herein or the pharmaceutical composition as taught herein for use in the treatment of an immune related disorder, preferably wherein the immune related disorder is an immune related disorder selected from the group consisting of cancer, inflammation, an infectious disease, an autoimmune disorder, allergy, organ transplant rejection, and graft-versus-host disease (GVH).

A further aspect provides the fusion protein as taught herein, the nucleic acid encoding the fusion protein as taught herein or the pharmaceutical composition as taught herein when comprising an immunomodulatory biomolecule, wherein the immunomodulatory biomolecule is a protein that enhances or suppresses an immune response, for use in targeting said immunomodulatory biomolecule to a target cell.

As supported by the example section, present inventors found that a fusion protein of an apolipoprotein or an apolipoprotein mimetic, preferably ApoA1, with IL-4 allows targeting IL-4 to the myeloid compartment. To their surprise, present inventors found that IL-4 can simultaneously reduce inflammation and induce trained immunity, particularly when targeted to the myeloid compartment. Therefore, the present inventors concluded that fusion proteins of an apolipoprotein or an apolipoprotein mimetic, preferably ApoA1, with IL-4 can be used to prevent immune related disorders by promoting trained immunity. Furthermore, present inventors found that IL-4's pharmacokinetic profile and bioavailability to innate immune cells can be further improved by integrating said fusion proteins into myeloid cell-avid lipid nanoparticles. Incorporation of such IL-4 fusion protein into a lipid nanoparticle does not hamper its ability to target to the myeloid compartment.

Accordingly, a further aspect of the invention provides a fusion protein comprising an apolipoprotein or an apolipoprotein mimetic and interleukin-4 (IL-4).

A further aspect of the invention provides a nucleic acid encoding the fusion protein comprising an apolipoprotein or an apolipoprotein mimetic and interleukin-4 (IL-4) as taught herein.

A further aspect of the invention provides a pharmaceutical composition comprising the fusion protein comprising an apolipoprotein or an apolipoprotein mimetic and interleukin-4 (IL-4) as taught herein or the nucleic acid encoding said fusion protein as taught herein, and a pharmaceutically acceptable carrier.

A further aspect of the invention provides the fusion protein comprising an apolipoprotein or an apolipoprotein mimetic and interleukin-4 (IL-4) as taught herein, the nucleic acid encoding such fusion protein as taught herein or the pharmaceutical composition comprising such fusion protein or nucleic acid for use as a medicament.

A further aspect of the invention provides the fusion protein comprising an apolipoprotein or an apolipoprotein mimetic and interleukin-4 (IL-4) as taught herein, the nucleic acid encoding such fusion protein as taught herein or the pharmaceutical composition comprising such fusion protein or nucleic acid for use in the treatment of an immune related disorder, preferably wherein the immune related disorder is a state of hyperinflammation followed by immune paralysis, preferably wherein the hyperinflammation and/or the immune paralysis is caused by an infectious disease such as COVID-19, by sepsis, myocardial infarction, stroke, cancer, or multiple sclerosis.

A further aspect of the invention provides the fusion protein comprising an apolipoprotein or an apolipoprotein mimetic and interleukin-4 (IL-4) as taught herein, the nucleic acid encoding such fusion protein as taught herein or the pharmaceutical composition comprising such fusion protein or nucleic acid for use in targeting IL-4 to a target cell, preferably a myeloid cell.

A further aspect of the invention provides a fusion protein comprising a myeloid-targeting molecule and IL-4, wherein the myeloid-targeting molecule is capable of targeting the IL-4 to a myeloid cell.

A further aspect of the invention provides a nucleic acid encoding the fusion protein comprising a myeloid-targeting molecule and IL-4 as taught herein.

A further aspect of the invention provides a nucleic acid comprising a nucleic acid sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 44 or comprising a nucleic acid sequence encoding a polypeptide having a sequence at least 90%, at least 91%, at least 92%, at least 93%, at least 94%, at least 95%, at least 96%, at least 97%, at least 98%, at least 99% or 100% identical to SEQ ID NO. 43 and further comprising means for targeted expression in a myeloid cell, wherein said mean are selected from:
a promoter for selective or inducible expression in said myeloid cell operatively linked to said nucleic acid; or
a viral expression vector comprising said nucleic acid capable of stably expressing said nucleic acid in said myeloid cell; or
a lipid nanoparticle comprising one or more apolipoproteins, phospholipids, said nucleic acid, and optionally sterol.

A further aspect of the invention provides a pharmaceutical composition comprising the fusion protein comprising a myeloid-targeting molecule and IL-4 as taught herein or the nucleic acid encoding said fusion protein as taught herein or the nucleic acid comprising means for targeted expression in a myeloid cell as taught herein, and a pharmaceutically acceptable carrier.

A further aspect of the invention the fusion protein comprising a myeloid-targeting molecule and IL-4 as taught herein or the nucleic acid encoding said fusion protein as taught herein or the nucleic acid comprising means for targeted expression in a myeloid cell as taught herein, or the pharmaceutical composition comprising said fusion protein or nucleic acid as taught herein for use as a medicament.

A further aspect of the invention provides the fusion protein comprising a myeloid-targeting molecule and IL-4 as taught herein or the nucleic acid encoding said fusion protein as taught herein or the nucleic acid comprising means for targeted expression in a myeloid cell as taught herein, or the pharmaceutical composition comprising said fusion protein or nucleic acid as taught herein for use in the treatment of an immune related disorder.

A further aspect of the invention provides in vivo, in vitro or ex vivo use of IL-4 in stimulating or promoting trained immunity in a cell, organ, tissue or an organism.

### BRIEF DESCRIPTION OF THE FIGURES

FIG. 1 depicts a schematic overview of the optional assembly of an exemplary apolipoprotein fusion in a lipid nanoparticle (sphere or disk) and subsequent binding to a target cell.

FIG. 2 depicts a schematic overview of different envisioned apolipoproteins and subsequent assembly in lipid nanoparticles. Depicted are the following fusion proteins (schematically): top left depicts both an apolipoprotein fused to an immunomodulatory biomolecule, and an apolipoprotein fused to a rerouting molecule; bottom left depicts an apolipoprotein fused to an immunomodulatory biomolecule; top right depicts an apolipoprotein fused to a rerouting molecule; bottom right depicts an apolipoprotein fused to an immunomodulatory biomolecule and a rerouting molecule.

FIG. 3 shows SDS-PAGE gels demonstrating the expression and purification of different apolipoprotein fusion constructs (ApoA1-IL1B, ApoA1-IL38, ApoA1-IL2). The rectangles indicate the bands corresponding to the desired proteins. (P: pellet containing cell debris, SN: supernatant containing soluble protein fraction, FT: flow through of SN applied to Ni-NTA column, A: first wash using 10 mM imidazole, A50: second wash using 50 mM imidazole, E1: elution fraction 1, E2: elution fraction 2, E3: elution fraction 3, E4: elution fraction 4, FW: final wash using 500 mM imidazole.)

FIG. 4 shows results of dynamic light scattering measurements for four lipid nanoparticles, three of which each contain a different apolipoprotein (ApoA1-IL1B, ApoA1-IL38, ApoA1-IL4) and one containing apoA1 as a control nanoparticle. Mean number diameter (darker grey) and polydispersity index (PdI) (lighter grey) have been determined for these nanoparticles over the course of 11 days.

FIG. 5 shows an SDS-PAGE gel demonstrating the expression and purification of apoA1 in which serine to cysteine mutations have been introduced, at either position 147 or 279. The rectangle indicates the band corresponding to the mutated apoA1.

FIG. 6 shows the quadrupole time-of-flight (Q-ToF) results for the apoA1 mutants. In both graphs, the chromatogram is plotted in the upper right corner, with below this the m/z values of the main peak from the chromatogram. The deconvoluted mass spectra show the presence of the desired mutant apoA1 proteins.

FIG. 7 shows the HPLC-MS chromatogram of interleukin (IL)-4 which has been modified to contain one N-terminal azide. The mass corresponding to the proteins represented by the peaks in the chromatogram are indicated.

FIG. 8 shows SDS-PAGE gels demonstrating the purity of the used IL-4, the IL-4 modified with an N-terminal azide (reaction at 4° C. or 20° C.), apoA1 coupled to a PEG-linker containing DBCO group, and the reaction products of coupling IL-4 modified to contain an azide to apoA1 with linker and DBCO group (reaction at 4° C. or 20° C.). The rectangle indicates the bands corresponding to the desired conjugation product.

FIG. 9 shows the results of a HEK293 IL-4 reporter cell assay in which the binding of commercially obtained IL-4 (mammalian), recombinantly expressed IL-4 (bacterial), recombinantly expressed apoA1-IL4 fusion protein, and chemically conjugated apoA1-IL4 fusion protein are evaluated. The absorbance corresponds to the level of binding of IL-4 to its receptor.

FIG. 10 shows an SDS-PAGE analysis of the chemical (right panel) and recombinant (left panel) apoA1-IL2 fusion constructs (apoA1-IL2 wild-type “ApoA1-IL2” or apoA1-IL2 mutant “ApoA1-IL2v4”). The rectangle indicates the band corresponding to the apoA1-IL2 or apoA1-IL2v4 fusion construct, respectively.

FIG. 11 shows the successful formulation of discoidal nanoparticles comprising apoA1-IL2 fusion proteins using cryogenic transmission electron microscopy (cryo-TEM) (Right panel) and the analysis of nanoparticle size and stability in PBS for 21 days using dynamic light scattering (DLS) (Left panel).

FIG. 12 shows the ability of the apoA1-IL2 fusion proteins to stimulate CD4+ or CD8+ T-cell proliferation. Abbreviations: PHA, phytohemagglutinin.

FIG. 13 shows an SDS-PAGE analysis of the chemical (right panel) and recombinant (left panel) apoA1-IL1p fusion constructs. The arrow (left panel) or rectangle (right panel) indicates the band corresponding to the apoA1-IL1p fusion construct.

FIG. 14 shows the successful formulation of discoidal nanoparticles comprising apoA1-IL1p fusion proteins using cryogenic transmission electron microscopy (cryo-TEM) (Right panel) and the analysis of nanoparticle size and stability in PBS for 21 days using dynamic light scattering (DLS) (Left panel).

FIG. 15 shows an SDS-PAGE analysis of the chemical (lower panel) and recombinant (upper panel) apoA1-IL38 fusion constructs. The arrow (upper panel) or rectangle (lower panel) indicates the band corresponding to the apoA1-IL38 fusion construct.

FIG. 16 shows the successful formulation of discoidal nanoparticles comprising apoA1-IL38 fusion proteins using cryogenic transmission electron microscopy (cryo-TEM) (Right panel) and the analysis of nanoparticle size and stability in PBS for 21 days using dynamic light scattering (DLS) (Left panel).

FIG. 17 IL4 inhibits acute inflammation, yet induces trained immunity. (A) Schematic of in vitro direct inflammation experiments. (B) TNF, IL6, and IL1Ra levels after 24 h stimulation of human primary monocytes. (C) Schematic of in vitro trained immunity experiments. (D) TNF and IL6 levels after re-stimulation of β-glucan-trained cells. (E) TNF and IL6 levels after restimulation of IL4-trained cells. (F) Seahorse analysis of glycolytic (left) and mitochondrial (right) metabolism in IL4-trained cells. Data are presented as mean±SD.

FIG. 18 Immune and epigenetic mechanisms mediating IL4-induced trained immunity. (A) Schematic overview of previously described premier IL4 signaling pathways. (B) TNF and IL6 levels after 24 h stimulation of monocytes whilst blocking key IL4 signaling routes. (C) TNF and IL6 levels after re-stimulation of cells that were trained with IL4 whilst blocking key IL4 signaling routes. (D) Heatmap of the transcriptome of IL4-trained cells, before and after re-stimulation. (E) Transcription factor motif enrichment analysis in IL4 trained immunity (heatmap indicates z-scores). (F) Pathway enrichment analyses of the IL4 trained immunity transcriptome. (G) TNF and IL6 levels after re-stimulation of cells that were trained with IL4 in the presence of a SET7 methyltransferase inhibitor. (H) ChIP-qPCR AUC analysis of TNF in IL4-trained cells. Data in bar graphs are presented as mean±SD.

FIG. 19 Engineering apoA1-IL4 fusion protein. (A) Schematic overview of apoA1-based fusion protein platform. (B) Schematic of apoA1-IL4 fusion protein structure. (C) SDS-PAGE and (D) Western blot of recombinantly expressed proteins. Antibodies specific for endogenous IL4 and apoA1. (E) Chromatogram and Q-TOF-MS spectrum of apoA1-IL4. (F) Kinetics of apoA1-IL4 binding to IL4Rα using SPR. (G) Activation of HEK-Blue cells expressing IL4Rα and IL13Rα1 by apoA1-IL4. Data are presented as mean±SD.

FIG. 20 Integrating apoA1-IL4 in nanoparticles platform. (A) Schematic representation of discoidal (upper panel) and spherical IL4-aNPs (lower panel) and (B) cryoTEM images. (C) IL4-aNP size distribution and (D) stability of IL4-aNPs over time as determined by the dynamic light scattering. IL4-aNP size is reported as the number mean. (E) Super-resolution fluorescence microscopy (dSTORM) images of human monocytes incubated with either fluorescently-labeled apoA1(-IL4) or (IL4-)aNPs and stained with anti-IL4Rα antibody. The co-localization between the proteins and IL4Rα can be appreciated by the arrows. White regions of interest are magnified in subsequent images on the right. Data are presented as mean±SD.

FIG. 21 Immunological in vitro, in vivo and ex vivo therapeutic evaluation of IL4-aNPs. (A) Schematic overview of the direct inflammation and trained immunity experiments in vitro. (B) TNF and IL6 levels after 24 h stimulation of monocytes in the presence of IL4-aNPs. (C) TNF and IL6 levels after restimulation of IL4(-aNP)-trained cells. (D) Schematic overview of murine in vivo tolerance model, including IL4-nanotherapy. (E) Serum TNF and IL6 levels following LPS re-challenge of mice treated IL4m-aNPs. The Mann-Whitney U test was used for statistical comparisons. (F) Schematic overview of human experimental endotoxemia model, including ex vivo tolerance reversal. (G) TNF and IL6 levels after ex vivo re-stimulation of human in vivo LPS-tolerized cells. (H) TNF and IL6 fold increase after ex vivo re-stimulation of human in vivo LPS-tolerized cells. Data are presented as mean±SD.

FIG. 22 depicts a schematic overview of the optional assembly of an exemplary apolipoprotein fusion comprising an apolipoprotein and a rerouting protein in a lipid nanoparticle (disc).

FIG. 23 shows the expression of VHHCD8-apoA1 fusion protein in Clearcoli cells. Minor protein contaminants are present after IMAC purification [lane E1]. The most prominent band corresponds to the fusion protein with a molecular weight of 43.3 kDa (rectangle).

FIG. 24 shows the successful formulation of discoidal nanoparticles comprising VHHCD8-apoA1 fusion proteins using cryogenic transmission electron microscopy (cryo-TEM) (Right panel) and the analysis of nanoparticle size and poly dispersity index (PDI) for 14 days using dynamic light scattering (DLS) (Left panel).

FIG. 25 shows the mean fluorescence intensity (MFI) of fluorescently labelled VHHCD8-apoA1 and apoA1 in mouse splenocytes (upper panel: CD3+ T cells from splenocytes; lower panel: all cells from the spleen).

FIG. 26 shows the mean fluorescence intensity (MFI) of discoidal and spherical aNPs formulated with VHHCD8-apoA1 and apoA1 and comprising a fluorescent dye in the lipid structure of the particle in mouse splenocytes.

FIG. 27 In vivo pharmacokinetics, biodistribution and safety profile after intravenous injection. (A) PET/CT 2 render at 24 h after injecting ⁸⁹Zr-labeled constructs. (B)⁸⁹Zr-labeled construct blood half-life (n=5, as fitted with a two-phase decay function). (C) Ex vivo gamma counting of tissues 24 h after ⁸⁹Zr-labeled construct injection (n=5), number represents ratio target to clearance organs. (D) Cell type-specific biodistribution of DiO-labeled discoid IL4-aNPs in spleen and bone marrow, as measured by flow cytometry. (E) ⁸⁹Zr-IL4-aNP blood half-life in non-human primates. (F) Organ SUVmean over time in ⁸⁹Zr-IL4-aNPs injected non-human primates (n=2). (G) Organ specific SUVmean 48 h after ⁸⁹Zr-IL4-aNPs injection in non-human primates (n=2). (H) PET/MRI scan of non-human primate 48 h after ⁸⁹Zr-IL4-aNPs injection. Data are presented as mean±SD where appropriate.

### DETAILED DESCRIPTION OF THE INVENTION

The recitation of numerical ranges by endpoints includes all integer numbers and, where appropriate, fractions subsumed within the respective ranges, as well as the recited endpoints. This applies to numerical ranges irrespective of whether they are introduced by the expression “from . . . to . . . ” or the expression “between . . . and . . . ” or another expression. Any numerical range recited herein is intended to include all sub-ranges subsumed therein.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

Similarly, it should be appreciated that in the description of illustrative embodiments of the invention, various features of the invention are sometimes grouped together in a single embodiment, figure, or description thereof for the purpose of streamlining the disclosure and aiding in the understanding of one or more of the various inventive aspects.

As used herein, the singular form terms “A,” “an,” and “the” include plural referents unless the content clearly dictates otherwise. Thus, for example, reference to “a cell” includes a combination of two or more cells, and the like.

As used herein, the term “and/or” refers to a situation wherein one or more of the stated cases may occur, alone or in combination with at least one of the stated cases, up to with all of the stated cases. For example, if a list is described as comprising group A, B, and/or C, the list can comprise A alone, B alone, C alone, A and B in combination, A and C in combination, B and C in combination, or A, B, and C in combination.

As used herein, the term “antigen” refers to a substance to which a binding portion of an antibody may bind. The specific immunoreactive sites within the antigen are known as “epitopes” (or antigenic determinants). A target for an antibody, or antigen-binding portion thereof, may comprise an antigen, such as is defined herein.

As used herein, the term “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . −10, −11, etc. Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

As used herein, the word “comprise” or variations thereof such as “comprises” or “comprising” will be understood to include a stated element, integer or step, or group of elements, integers or steps, but not to exclude any other element, integer or steps, or groups of elements, integers or steps. The verb “comprising” includes the verbs “essentially consisting of” and “consisting of”.

As used herein, the term “conventional techniques” refers to a situation wherein the methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al., Molecular Cloning. A Laboratory Manual, 2nd Edition, Cold Spring Harbor Laboratory Press, Cold Spring Harbor, N. Y., 1989; Ausubel et al., Current Protocols in Molecular Biology, John Wiley & Sons, New York, 1987 and periodic updates; and the series Methods in Enzymology, Academic Press, San Diego.

As used herein, the term “identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: (Computational Molecular Biology, Lesk, A. M., ED., Oxford University Press, New York, 1988; Biocomputing: Informatics And Genome Projects, Smith, D. W., ED., Academic Press, New York, 1993; Computer Analysis Of Sequence Data, Part I, Griffin, A. M., And Griffin, H. G., EDS., Humana Press, New Jersey, 1994; Sequence Analysis In Molecular Biology, Von Heinje, G., Academic Press, 1987; and Sequence Analysis Primer; Gribskov, M. and Devereux, J., eds., M Stockton Press, New York, 1991). While there exist a number of methods to measure identity between two nucleotide sequences or amino acid sequences, the term “identity” is well known to skilled artisans (Carillo, H., and Lipton, D., SIAM J. Applied Math (1988) 48:1073). Methods commonly employed to determine identity or similarity between two sequences include, but are not limited to, those disclosed in Guide To Huge Computers, Martin J. Bishop, ed., Academic Press, San Diego, 1994, and Carillo, H., and Lipton, D., Siam J. Applied Math (1988) 48:1073. Methods to determine identity and similarity are codified in computer programs. Preferred computer program methods to determine identity and similarity between two sequences include, but are not limited to, GCS program package (Devereux, J., et al., Nucleic Acids Research (1984) 12(1):387), BLASTP, BLASTN, FASTA (Atschul, S. F. et al., J. Molec. Biol. (1990) 215:403).

As an illustration, by a polynucleotide having a nucleotide sequence having at least, for example, 95% “identity” to a reference nucleotide sequence encoding a polypeptide of a certain sequence, it is intended that the nucleotide sequence of the polynucleotide is identical to the reference sequence except that the polynucleotide sequence may include up to five point mutations per each 100 nucleotides of the reference amino acid sequence. In other words, to obtain a polynucleotide having a nucleotide sequence at least 95% identical to a reference nucleotide sequence, up to 5% of the nucleotides in the reference sequence may be deleted and/or substituted with another nucleotide, and/or a number of nucleotides up to 5% of the total nucleotides in the reference sequence may be inserted into the reference sequence. These mutations of the reference sequence may occur at the 5′ or 3′ terminal positions of the reference nucleotide sequence, or anywhere between those terminal positions, interspersed either individually among nucleotides in the reference sequence or in one or more contiguous groups within the reference sequence.

Similarly, by a polypeptide having an amino acid sequence having at least, for example, 95% “identity” to a reference amino acid sequence of SEQ ID NO: X is intended that the amino acid sequence of the polypeptide is identical to the reference sequence except that the amino acid sequence may include up to five amino acid alterations per each 100 amino acids of the reference amino acid of SEQ ID NO: X. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As used herein, the term “in vitro” refers to experimentation or measurements conducted using components of an organism that have been isolated from their natural conditions.

As used herein, the term “ex vivo” refers to experimentation or measurements done in or on tissue from an organism in an external environment with minimal alteration of natural condition.

As used herein, the term “nucleic acid”, “nucleic acid molecule” and “polynucleotide” is intended to include DNA molecules and RNA molecules. A nucleic acid (molecule) may be single-stranded or double-stranded, but preferably is double-stranded DNA.

As used herein, the terms “sequence” when referring to nucleotides, or “nucleic acid sequence”, “nucleotide sequence” or “polynucleotide sequence” refer to the order of nucleotides of, or within, a nucleic acid and/or polynucleotide. Within the context of the current invention a first nucleic acid sequence may be comprised within or overlap with a further nucleic acid sequence.

As used herein, the term “subject” or “individual” or “animal” or “patient” or “mammal,” used interchangeably, refer to any subject, particularly a mammalian subject, for whom diagnosis, prognosis, or therapy is desired. Mammalian subjects include humans, domestic animals, farm animals, and zoo-, sports-, or pet-animals such as dogs, cats, guinea pigs, rabbits, rats, mice, horses, cattle, cows, bears, and so on. As defined herein a subject may be alive or dead. Samples can be taken from a subject post-mortem, i.e. after death, and/or samples can be taken from a living subject.

As used herein, terms “treatment”, “treating”, “palliating”, “alleviating” or “ameliorating”, used interchangeably, refer to an approach for obtaining beneficial or desired results including, but not limited to, therapeutic benefit. By therapeutic benefit is meant eradication or amelioration or reduction (or delay) of progress of the underlying disease being treated. Also, a therapeutic benefit is achieved with the eradication or amelioration or reduction (or delay) of progress of one or more of the physiological symptoms associated with the underlying disease such that an improvement or slowing down or reduction of decline is observed in the patient, notwithstanding that the patient can still be afflicted with the underlying disease.

As used herein the term “vector” refers to a nucleic acid molecule capable of transporting another nucleic acid to which the nucleic acid molecule capable of transporting has been linked. One type of vector is a “plasmid”, which refers to a circular double stranded DNA loop into which additional DNA segments may be ligated. The term “vector” may also refer to the viral particle (i.e. viral vector) which contains the nucleic acid of interest.

A portion of this invention contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent invention, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention relates, unless otherwise indicated. Other specifically defined terms are to be construed in a manner consistent with the definition as provided herein. The preferred materials and methods are described herein, although any methods and materials similar or equivalent to those described herein can be used in the practice for testing of the present invention.

Unless otherwise defined, all technical and scientific terms used herein have the same meaning as commonly understood by a person of ordinary skill in the art.

The present invention is based on the inventors' finding that apolipoprotein or an apolipoprotein mimetic can be used as a carrier for therapeutic agents, and that apolipoproteins may further be modified to target specific cells, tissues or organs. The inventors found that fusion proteins of cytokines with apolipoproteins or apolipoprotein mimetics demonstrate strongly increased half-life in blood, thereby opening up the possibility to use cytokines in a therapeutical manner without requiring toxic concentrations to be administered. Further it was realized that apolipoproteins or apolipoprotein mimetics allow targeting of cytokines when fused together, such as to a target cell, tissue and/or organ, like a myeloid cell. The inventors realized that this concept is more broadly applicable and can also be used for a broad spectrum of immunomodulatory biomolecules such as cytokines, chemokines, hormones, growth factors etc.

It was further realized that it is possible to direct a fusion protein or apolipoprotein or an apolipoprotein mimetic to an intended target by linking it to a rerouting molecule. In this way the apolipoprotein (or the apolipoprotein mimetic) or the fusion protein can be targeted to cells, tissues or organs it would otherwise not or insufficiently reach, or it could be used to reduce off-target effects.

Therefore, in a first aspect, the invention relates to a fusion protein comprising, consisting essentially of, or consisting of an apolipoprotein or an apolipoprotein mimetic and an immunomodulatory biomolecule, wherein the immunomodulatory biomolecule is a protein that modulates (e.g. enhances or suppresses) an immune response.

In a second aspect the invention relates to a fusion protein comprising, consisting essentially of, or consisting of an apolipoprotein or an apolipoprotein mimetic and a rerouting molecule, wherein the rerouting molecule is a molecule that allows, when fused to the apolipoprotein, the apolipoprotein to bind to a different target than it would bind when the apolipoprotein was not fused to the rerouting molecule and/or to bind to its intended target with a higher affinity.

In a third aspect the invention relates to a fusion protein comprising, consisting essentially of, or consisting of an apolipoprotein or an apolipoprotein mimetic and an immunomodulatory biomolecule and a rerouting molecule.

The fusion protein may be conveniently denoted as being a fusion protein of an apolipoprotein or an apolipoprotein mimetic with an immunomodulatory biomolecule and/or a rerouting molecule.

The fusion proteins may be used as such, meaning not as part of a lipoprotein, lipid or apolipoprotein lipid nanoparticle. In such way the fusion protein may serve as a carrier to deliver an immunomodulatory biomolecule to a target site. Alternatively, the rerouting molecule can be used to target the fusion protein to a specific site, such as a cell, tissue, or organ.

Apolipoproteins are proteins that bind lipids such as triglycerides and cholesterol to form lipoproteins. They transport lipids (and fat soluble vitamins) in blood, cerebrospinal fluid and lymph. The lipid components of lipoproteins are insoluble in water. However, because of their detergent-like (amphipathic) properties, apolipoproteins and other amphipathic molecules (such as phospholipids) can surround the lipids, creating a lipoprotein particle that is itself water-soluble, and can thus be carried through water-based circulation (i.e., blood, lymph). In addition to stabilizing lipoprotein structure and solubilizing the lipid component, apolipoproteins interact with lipoprotein receptors and lipid transport proteins, thereby participating in lipoprotein uptake and clearance.

In lipid transport, apolipoproteins function as structural components of lipoprotein particles, ligands for cell-surface receptors and lipid transport proteins, and cofactors for enzymes. Different lipoprotein particles contain different classes of apolipoproteins, which influence their function. For example, apolipoprotein A-I (apoA1) is the major structural protein component of high-density lipoproteins (HDL), although it is present in other lipoproteins in smaller amounts, and HDL comprises other apolipoproteins.

It is envisioned that the invention is not limited to a particular type of apolipoprotein or apolipoprotein mimetic (e.g. ApoA1, ApoB or ApoE). Therefore in an embodiment, the apolipoprotein of the fusion protein of the invention, such as the apolipoprotein of the fusion protein according to the first, second or third aspect of the invention is selected from apoA1, ApoA-1 Milano, apoA2, apoA4, apoA5, apoB48, apoB100, apoC-1, apoC-II, apoC-III, apoC-IV, apoD, apoE, apoF, apoH, apoL1, apoL2, apoL3, apoL4, apoL5, apoL6, apoLD1, apoM, apoO, apoOL, or combinations thereof, or a mimetic thereof. For example, the apolipoprotein may be selected from apoA1, ApoA-1 Milano, apoA2, apoA4, apoA5, apoB48, apoB100, apoC-1, apoC-II, apoC-Ill, apoC-IV, apoE, apoL1, apoL2, apoL3, apoL4, apoL5, apoL6, or combinations thereof or mimetics thereof. More preferably the apolipoprotein may be selected from apoA1, apoA4, apoC3, apoD, apoE, apoL1, apoL3, or combinations thereof or mimetics thereof. In other words, in particular embodiments, the apolipoprotein is an ApoA1, ApoA-1 Milano, ApoA4, ApoC3, ApoD, ApoE, ApoL1, ApoL3 or the apolipoprotein mimetic is a mimetic of an ApoA1, ApoA-1 Milano, ApoA4, ApoC3, ApoD, ApoE, ApoL1, ApoL3. Even more preferably, the apolipoprotein is ApoA1.

In particular embodiments, the apolipoprotein may also be an apolipoprotein fragment. Preferably, the apolipoprotein fragment retains the biological activity of the apolipoprotein, such as the ability of the apolipoprotein to integrate into a lipid nanoparticle or to target an immunomodulatory biomolecule to a target, such as to the myeloid compartment. In particular embodiments, the apolipoprotein fragment comprises at least the ATP Binding Cassette Subfamily A Member 1 (ABCA1), ATP Binding Cassette Subfamily G Member 1 (ABCG1) and/or Scavenger receptor class B type 1 (SR-BI) binding regions of the full-length apolipoprotein, thereby allowing binding to a myeloid cell. In particular embodiments, the apolipoprotein fragment comprises at least the alpha helices of the full-length apolipoprotein. These helices are hydrophilic on one side (interact with aqueous environment) and hydrophobic (interacts with lipids in the particle) on the other side.

The term “fragment” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, or ≥200, consecutive amino acids of the corresponding full-length peptide, polypeptide, or protein.

In particular embodiments, the apolipoprotein fragment comprises the myeloid-binding portion of full-length apolipoprotein.

In particular embodiments, the apolipoprotein may also be an apolipoprotein mutant comprising a mutation that allows chemical conjugation of the apolipoprotein to an immunomodulatory biomolecule and/or a rerouting molecule. In particular embodiments, the apolipoprotein may also be an apolipoprotein mutant comprising a serine to cysteine substitution, such as the ApoA1 mutant as defined by SEQ ID NO: 1, 7, 9 or 11 as described elsewhere herein.

Peptide sequences for the different proteins described herein, or nucleic acid sequences for the genes encoding the different proteins described herein, are readily available to the skilled person, for example from the UCSC Genome Browser (http://genome.ucsc.edu/), Ensembl genome browser (https://www.ensembl.org) and NCBI (https://www.ncbi.nlm.nih.gov/protein). Consensus sequences for different proteins or genes are readily derived from these sources, although it is understood a certain variation may be present due to but not limited to genetic variation and multiple splice variants of the gene. Therefore, when referring to a specific protein, this should be interpreted to encompass sequence variations due to genetic variation and splice variants. Therefore, when used herein when referring to a certain protein, this should be interpreted as the corresponding consensus protein sequence as retrieved from the Ensembl genome browser, or the consensus nucleic acid (gene) sequence as retrieved from the Ensembl genome browser, or a protein sequence 80%, 85%, 90%, 91%, 92%, 93%, 94%, 95%, 96%, 97%, 98%, 99% or 100% identical to the corresponding consensus protein sequence as retrieved from the Ensembl genome browser, or a gene sequence 70%, 75%, 80%, 85%, 90%, 91%, 92%, 93%, 94%, 95%, 96%, 97%, 98%, 99% or 100% identical to the corresponding consensus gene sequence as retrieved from the Ensembl genome browser, or a nucleic acid sequence encoding a protein 80%, 85%, 90%, 91%, 92%, 93%, 94%, 95%, 96%, 97%, 98%, 99% or 100% identical to the corresponding consensus protein sequence as retrieved from the Ensembl genome browser.

Apolipoprotein mimetics are synthetic peptides or proteins that mimic the function or structure of apolipoproteins. Several apolipoprotein mimetics are known and for example Wolska et al. (Cells. 2021 March; 10(3): 597., incorporated by reference in its entirety) review different apoA1, apoE and apoC-II mimetics described in the literature. For example, ApoA1 mimetic peptides have largely been designed based on their ability to efflux cholesterol from cells. As this process has not been shown to depend upon a specific protein-protein interaction, most apoA1 mimetic peptides are simply just amphipathic helices and, in fact, many have no primary amino acid homology to apoA1. Exemplary ApoA1 mimetics are ApoA1 mimetic 18A, ApoA1 mimetic 2F and ApoA1 mimetic 37 pA, which are represented by the peptides sequences corresponding to SEQ ID Nos 51, 52 and 53.

For example, apoE has several putative atheroprotective functions, many different types of apoE-based peptides have been reported. One of the main goals in the design of these peptides is to facilitate the hepatic clearance of apoB-containing lipoproteins. As apoE can only bind to its receptor when bound to lipids, these peptides usually have not only the receptor-binding motif from the N-terminal domain of apoE, but also a lipid-binding region based on the C-terminal domain of apoE or some other sequence.

For example apoC-II mimetics have been described either based on a shortened first helix (18A) linked to the LPL-activation domain of apoC-II, or mimetics where both the first and second helix are based on the native apoC-II helices with amino acid substitutions to enhance bihelical binding to lipoproteins.

Therefore, when used herein, an apolipoprotein mimetic refers to a synthetic protein or peptide which shares a structural and/or functional feature with the respective apolipoprotein. For example, the shared structural feature may be a primary, secondary or tertiary peptide structure such as the peptide sequence, presence of structures such as an alpha helix or beta sheet or three-dimensional structure of the peptide, or the functional feature may be a similarity in binding to a certain target such as a receptor. Preferably the apolipoprotein is capable of binding lipids, more preferably forming lipid particles, in a similar manner as the corresponding apolipoprotein.

In particular embodiments, the apolipoprotein mimetic is able to bind to a myeloid cell to the same or a similar extent as the respective apolipoprotein. For example, the apolipoprotein mimetic of ApoA1 is able to bind to a myeloid cell to the same or a similar extent as ApoA1.

In an embodiment the fusion protein is a fusion protein of ApoA1 (or a mutant thereof), preferably human ApoA1, with an immunomodulatory biomolecule and/or a rerouting molecule.

By means of an example, the human ApoA1 protein sequence is annotated under NCBI Genbank (http://www.ncbi.nlm.nih.gov/) accession number NP_001304947.1 (isoform 1 preproprotein), and Uniprot (www.uniprot.org) accession number P02647.1.

In particular embodiments, the ApoA1 is wild-type ApoA1 (e.g. as derived from the human precursor of ApoA1 as defined by SEQ ID NO. 1, of which the first 18 amino acids form the signal peptide) or an ApoA1 mutant (e.g. as defined by SEQ ID NO. 7, 9 or 11). In particular embodiments, the ApoA1 is wild-type human ApoA1 as defined by SEQ ID NO. 78.

For example, in order to chemically conjugate apoA1 to an immunomodulatory biomolecule and/or a rerouting molecule, a reactive handle is typically needed. Therefore, apoA1 mutants comprising a cysteine in the place of a serine at position 147 (e.g. as defined by SEQ ID NO. 9) or 279 (e.g. as defined by SEQ ID NO: 11) could be useful to prepare chemically conjugated ApoA1 fusion proteins.

In an embodiment, the ApoA1 is a peptide with, or the ApoA1 comprises, consists essentially of or consists of, an amino acid sequence at least 90%, at least 91%, at least 92%, at least 93%, at least 94%, at least 95%, at least 96%, at least 97%, at least 98%, at least 99% or 100% identical to SEQ ID NO. 1, SEQ ID NO. 3, SEQ ID NO. 5, SEQ ID NO. 7, SEQ ID NO. 9 or SEQ ID NO. 11, or comprising, consisting essentially of or consisting of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 2, SEQ ID NO. 4, SEQ ID NO. 6, SEQ ID NO. 8, SEQ ID NO. 10 or SEQ ID NO. 12.

It is noted that the ApoA1 sequence as defined by SEQ ID NO. 3, SEQ ID NO. 7, SEQ ID NO. 9 or SEQ ID NO. 11 comprise N-terminally the amino acid sequence GLVPRGSIDD (SEQ ID NO. 79), which is a thrombin cleavage site. For example, the ApoA1 sequence as defined by SEQ ID NO. 5 comprises N-terminally a 6His tag followed by the amino acid sequence GLVPRGSIDD (SEQ ID NO. 79). Here, the thrombin cleavage site as could be used to remove the N-terminal His tag from the peptide.

In an embodiment, the ApoA1 is a peptide with, or the ApoA1 comprises, consists essentially of or consists of, an amino acid sequence at least 90%, at least 91%, at least 92%, at least 93%, at least 94%, at least 95%, at least 96%, at least 97%, at least 98%, at least 99% or 100% identical to SEQ ID NO. 7, wherein SEQ ID NO. 7 comprises a cysteine at position 7 of SEQ ID NO. 7. Such ApoA1 mutant may be referred to herein as “S14C” mutant.

In an embodiment, the ApoA1 is a peptide with, or the ApoA1 comprises, consists essentially of or consists of, an amino acid sequence at least 90%, at least 91%, at least 92%, at least 93%, at least 94%, at least 95%, at least 96%, at least 97%, at least 98%, at least 99% or 100% identical to SEQ ID NO. 9, wherein SEQ ID NO. 7 comprises a cysteine at position 150 of SEQ ID NO. 9. Such ApoA1 mutant may be referred to herein as “S147C” or “S157C” mutant.

In an embodiment, the ApoA1 is a peptide with, or the ApoA1 comprises, consists essentially of or consists of, an amino acid sequence at least 90%, at least 91%, at least 92%, at least 93%, at least 94%, at least 95%, at least 96%, at least 97%, at least 98%, at least 99% or 100% identical to SEQ ID NO. 11, wherein SEQ ID NO. 7 comprises a cysteine at position 239 of SEQ ID NO. 11. Such ApoA1 mutant may be referred to herein as “S279C” or “S239C” mutant.

In an embodiment the fusion protein is a fusion protein of an ApoA1 mimetic with an immunomodulatory biomolecule and/or a rerouting molecule. In an embodiment, the ApoA1 mimetic is a peptide with or the ApoA1 mimetic comprises, consists essentially of or consists of, an amino acid sequence at least 90%, at least 91%, at least 92%, at least 93%, at least 94%, at least 95%, at least 96%, at least 97%, at least 98%, at least 99% or 100% identical to SEQ ID NO. 51, SEQ ID NO. 52, or SEQ ID NO. 53.

In an embodiment the fusion protein is a fusion protein of ApoE with an immunomodulatory biomolecule and/or a rerouting molecule. In an embodiment, the ApoE is a peptide with or the ApoE comprises, consists essentially of or consists of, an amino acid sequence at least 90%, at least 91%, at least 92%, at least 93%, at least 94%, at least 95%, at least 96%, at least 97%, at least 98%, at least 99% or 100% identical to SEQ ID NO. 13, SEQ ID NO. 15, SEQ ID NO. 17 or SEQ ID NO. 19, or comprising, consisting essentially of or consisting of, an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 14, SEQ ID NO. 16, SEQ ID NO. 18 or SEQ ID NO. 20.

As described elsewhere herein, the fusion protein of ApoA1 may be a fusion protein of ApoA1 and a cytokine, such as IL-1β (IL-1B), IL-2, IL-4 or IL-38, preferably IL-4.

Accordingly, in an embodiment the fusion protein is a fusion protein of ApoA1 (including mutants thereof) with interleukin (IL)-1B, preferably human IL-1B.

In an embodiment, the ApoA1-IL1B fusion protein comprises or consists of a polypeptide with an amino acid sequence at least 90%, at least 91%, at least 92%, at least 93%, at least 94%, at least 95%, at least 96%, at least 97%, at least 98%, at least 99% or 100% identical to SEQ ID NO. 21 or 82, or comprising or consisting of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 22 or 83.

In an embodiment the fusion protein is a fusion protein of ApoA1 (including mutants thereof) with IL-2, preferably human IL-2.

In an embodiment, the ApoA1-IL2 fusion protein comprises or consists of a polypeptide with an amino acid sequence at least 90%, at least 91%, at least 92%, at least 93%, at least 94%, at least 95%, at least 96%, at least 97%, at least 98%, at least 99% or 100% identical to SEQ ID NO. 23, SEQ ID NO. 25, SEQ ID NO. 27, SEQ ID NO. 29, SEQ ID NO. 31, SEQ ID NO. 33, SEQ ID NO. 58 or SEQ ID NO. 60, or comprising or consisting of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 24, SEQ ID NO. 26, SEQ ID NO. 28, SEQ ID NO. 30, SEQ ID NO. 32,SEQ ID NO. 34, SEQ ID NO. 59 or SEQ ID NO. 61.

In an embodiment the fusion protein is a fusion protein of ApoA1 (including mutants thereof) with IL-4, preferably human IL-4.

In an embodiment, the ApoA1-IL4 fusion protein comprises or consists of a polypeptide with an amino acid sequence at least 90%, at least 91%, at least 92%, at least 93%, at least 94%, at least 95%, at least 96%, at least 97%, at least 98%, at least 99% or 100% identical to SEQ ID NO. 35, SEQ ID NO. 37 or SEQ ID NO. 39, or comprising or consisting of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 36, SEQ ID NO. 38 or SEQ ID NO. 40.

In an embodiment the fusion protein is a fusion protein of ApoA1 (including mutants thereof) with IL-38 (also known as IL1F10), preferably human IL-38.

In an embodiment, the ApoA1-1L38 fusion protein comprises or consists of a polypeptide with an amino acid sequence at least 90%, at least 91%, at least 92%, at least 93%, at least 94%, at least 95%, at least 96%, at least 97%, at least 98%, at least 99% or 100% identical to SEQ ID NO. 80 or 84, or comprising or consisting of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 81 or 85.

As described elsewhere herein, the ApoA1 may also be fused to a rerouting molecule, such as a rerouting molecule capable of binding to lymphocytes, preferably T cells, more preferably CD8+ T cells. For example, the rerouting molecule may be a VHHCD8 as described in Woodham A. W. et al., Nanobody-antigen conjugates elicit HPV-specific antitumor immune responses, Cancer Immunology Research, 2018, Vol. 6, issue 7, and comprising an amino acid sequence as shown in Supplemental Table 1 of said reference.

In an embodiment the fusion protein is a fusion protein of ApoA1 (including mutants thereof) with VHH8CD8.

In an embodiment, the VHHCD8-apoA1 fusion protein comprises or consists of a polypeptide with an amino acid sequence at least 90%, at least 91%, at least 92%, at least 93%, at least 94%, at least 95%, at least 96%, at least 97%, at least 98%, at least 99% or 100% identical to SEQ ID NO. 54 or 56, or comprising or consisting of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 55 or 57.

When used herein, fusion protein when referring to an apolipoprotein fusion protein should be interpreted as an apolipoprotein or apolipoprotein mimetic and covalently attached thereto an immunomodulatory biomolecule and/or a rerouting molecule. The covalent attachment may be due to the in frame coding of a peptide or protein sequence by the nucleotide sequence that encodes the fusion protein. Alternatively, the covalent attachment may be due to covalent linkage of the immunomodulatory biomolecule and/or a rerouting molecule to the apolipoprotein, for example via a sulfur bond such as a thioether bond, formed at a cysteine residue of the apolipoprotein. It is understood that the immunomodulatory biomolecule and/or the rerouting molecule and/or the apolipoprotein (or mimetic thereof) may include the site-specific incorporation of non-natural amino acids such as para-azidophenylalanine, which can be used in subsequent (strain-promoted) “click” (conjugation) reactions with alkyne modified reagents.

In particular embodiments, the fusion protein comprises a linker, such as a flexible linker, between the apolipoprotein or apolipoprotein mimetic and said immunomodulatory biomolecule and/or rerouting molecule. The linker may be a glycine-serine linker, such as a (GGS)n-linker, wherein n is 0, 1, 2, 3, 4, 5, 6, 7, 8, 9 or 10, preferably a (GGS)4-linker.

In particular embodiments, the fusion protein may comprise one or more tags, such as at the N- and/or C-terminal end of the fusion protein. The one or more tags, such as a 6His-tag or strep-tag may allow purification of the fusion protein.

Said immunomodulatory biomolecule and/or rerouting molecule may be covalently attached to any portion of the apolipoprotein or apolipoprotein mimetic. A linker, such as a flexible linker, may be used to allow such covalent attachment.

In particular embodiments, said immunomodulatory biomolecule is located N- or C-terminally of said apolipoprotein or apolipoprotein mimetic in said fusion protein.

In particular embodiments, said rerouting molecule is located N- or C-terminally of said apolipoprotein or apolipoprotein mimetic in said fusion protein.

In particular embodiments, wherein said fusion protein comprises an immunomodulatory biomolecule and a rerouting molecule, said apolipoprotein or apolipoprotein mimetic may be located N- or C-terminally of both said immunomodulatory biomolecule and said rerouting molecule or may be located in between said immunomodulatory biomolecule and said rerouting molecule (e.g. with the immunomodulatory biomolecule N- or C-terminally of the apolipoprotein or apolipoprotein mimetic).

When used herein an immune response refers to a reaction which occurs within an organism by the immune system. The immune response may be the innate immune response or the adaptive immune response or the complement immune system. Immune responses when referred herein include but are not limited to: secretion of a pro-inflammatory molecule; secretion of an anti-inflammatory molecule; phagocytosis; antibody production, presentation or secretion; antigen presentation; activation, proliferation, suppression or differentiation of an immune cell; binding of an immune cell to a target, initiation of an immune related cellular signaling cascade, or combinations thereof.

When used herein an immunomodulatory biomolecule refers to a molecule that enhances or suppresses an immune response. The biomolecule may interfere with, change, stimulate or suppress the innate immune response or the adaptive immune response or the complement immune system. The biomolecule may be a protein, peptide or organic compound. The compound may be isolated or derived from a natural source, cloned or synthesized. Non limiting examples of immunomodulatory biomolecules are cytokines, chemokines, hormones, growth factors and hematopoietic growth factors and antibodies (or antigen binding fragments thereof), although the skilled person may be aware of additional immunomodulatory molecules.

Therefore, in an embodiment the immunomodulatory biomolecule is selected from a cytokine, a chemokine, a hormone, a growth factor, a hematopoietic growth factor, an antibody or antigen binding fragment thereof, or combinations thereof.

In an embodiment the immunomodulatory biomolecule may be a cytokine. Cytokines are known to the skilled person to be small proteins of approximately 5 to 20 kDa and are important in cell signaling. For example, a cytokine may refer to: a four-alpha-helix bundle family cytokine, such as the interleukin (IL)-2 subfamily, the interferon (IFN) subfamily or the IL-10 subfamily; the IL-1 family; the cysteine knot cytokines such as the transforming growth factor (TGF) beta family; the IL-17 family. Therefore, the cytokine is preferably selected from IL18, interleukin 18 binding protein (IL18BP), interleukin 1 alpha (IL1A), interleukin 1 beta (IL1B), interleukin-1 family member 10 (IL1F10), IL1F3/IL1RA, IL1F5, IL1F6, IL1F7, IL1F8, interleukin 1 receptor like 2 (IL1RL2), IL1F9, IL33, B-cell activating factor (BAFF), 4-1 BBL, TNF super family member 8 (TNFSF8), cluster of differentiation 40 (CD40) ligand (CD40LG), CD70, CD95L/CD178, ectodysplasin-A1 (EDA-A1), TNFSF14, lymphotoxin alpha (LTA)/TNFB, lymphotoxin beta (LTB), TNFalpha, TNFSF10, TNFSF11, TNFSF12, TNFSF13, TNFSF15, TNFSF4, interferon alpha (IFNA)1, IFNA10, IFNA13, IFNA14, IFNA2, IFNA4, IFNA7, interferon beta (IFNB) 1, interferon epsilon (IFNE), interferon gamma (IFNG), interferon zeta (IFNZ), IFNA8, IFNA5/IFNaG, IFNω/IFNW1, cardiotrophin like cytokine factor 1 (CLCF1), ciliary neurotrophic factor (CNTF), IL11, IL31, IL6, Leptin, leukemia inhibitory factor (LIF), oncostatin M (OSM), IL10, IL19, IL20, IL22, IL24, IL28B, IL28A, IL29, TGF-beta 1/TGFB1, TGF-beta 2/TGFB2, TGF-beta 3/TGFB3. In a preferred embodiment the cytokine is selected from the IL-2 subfamily, the interferon subfamily, the IL-10 subfamily, the IL-1 family, the TGFbeta family, or the IL-17 family, or combinations thereof, more preferably wherein the cytokine is selected from IL-1β, IL-2, IL-4, IL-38, or combinations thereof. More preferably, wherein the cytokine is IL-4.

In particular embodiments, the cytokine is a human cytokine.

In an embodiment the fusion protein is a fusion protein of an apolipoprotein or a mimetic thereof with IL-1B. IL-1B is also known as IL1B, IL-1β, IL1F2 or interleukin 1 beta, and is a cytokine protein that in humans is encoded by the IL1B gene. By means of an example, the human IL-1B protein sequence is annotated under NCBI Genbank (http://www.ncbi.nlm.nih.gov/) accession number NP_000567.1 (proprotein), and Uniprot (www.uniprot.org) accession number P01584.2.

In an embodiment, the IL-1B is a peptide with, or comprising, consisting essentially of or consisting of, an amino acid sequence at least 90%, at least 91%, at least 92%, at least 93%, at least 94%, at least 95%, at least 96%, at least 97%, at least 98%, at least 99% or 100% identical to SEQ ID NO. 45, or comprising, consisting essentially of or consisting of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 46.

In an embodiment the fusion protein is a fusion protein of an apolipoprotein or a mimetic thereof with IL-2. IL-2 is also known as IL2 TCGF, lymphokine or interleukin 2, and is an interleukin that regulates the activities of leukocytes that are responsible for immunity. By means of an example, the human IL-2 protein sequence is annotated under NCBI Genbank (http://www.ncbi.nlm.nih.gov/) accession number NP_000577.2, and Uniprot (www.uniprot.org) accession number P60568.1.

In particular embodiments, IL-2 may be wild-type IL-2 or an IL-2 mutant.

In particular embodiments, IL-2 may comprise one or more amino acid substitutions as described in Wang A. et al., Science, Site-specific mutagenesis of the human interleukin-2 gene: structure-function analysis of the cysteine residues., 1984, Vol. 224, No. 4656 or Klein C. et al., Cergutuzumab amunaleukin (CEA-IL2v), a CEA-targeted IL-2 variant-based immunocytokine for combination cancer immunotherapy: Overcoming limitations of aldesleukin and conventional IL-2-based immunocytokines, Oncolmmunology, 2017, Volume 6, issue 3, e1277306.

In particular embodiments, IL-2 may comprise one or more, such as one, two, three or all four of the following amino acid substitutions: F42A, Y45A, L72G and/or C125A (the positions of the substitutions are indicated vis-à-vis the sequence of the mature human IL-2 protein). It is noted that the first 20 amino acids of the human IL-2 amino acid sequence as defined by SEQ ID NO. 47 (human IL-2 precursor) represent the signal peptide, accordingly, IL-2 may comprise one or more, such as one, two, three or all four of the following amino acid substitutions, F62A, Y65A, L92G and/or C145A, wherein the positions are indicated vis-à-vis SEQ ID NO. 47.

In an embodiment, the IL-2 is a peptide with, or comprising, consisting essentially of or consisting of, an amino acid sequence at least 90%, at least 91%, at least 92%, at least 93%, at least 94%, at least 95%, at least 96%, at least 97%, at least 98%, at least 99% or 100% identical to SEQ ID NO. 47, or comprising, consisting essentially of or consisting of, an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 48.

In an embodiment the fusion protein is a fusion protein of an apolipoprotein or a mimetic thereof with IL-4. IL-4 is also known as BSF-1, IL4 or interleukin 4, is a cytokine that induces differentiation of naive helper T cells. By means of an example, the human IL-4 protein sequence is annotated under NCBI Genbank (http://www.ncbi.nlm.nih.gov/) accession number NP_000580.1 (isoform 1 precursor), and Uniprot (www.uniprot.org) accession number P05112.1.

In an embodiment, the IL-4 is a peptide with, or comprising, consisting essentially of or consisting of, an amino acid sequence at least 90%, at least 91%, at least 92%, at least 93%, at least 94%, at least 95%, at least 96%, at least 97%, at least 98%, at least 99% or 100% identical to SEQ ID NO. 43, or comprising, consisting essentially of or consisting of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 44.

In a further embodiment, the fusion protein is a fusion protein of ApoA1 or a mimetic thereof with IL-4.

In an embodiment the fusion protein is a fusion protein of an apolipoprotein or a mimetic thereof with IL-38. IL-38 is also known as IL38, IL1F10 interleukin 38, interleukin 1 family member 10 or IL1-theta, and is a protein that in humans is encoded by the IL1F10 gene. By means of an example, the human IL-38 protein sequence is annotated under NCBI Genbank (http://www.ncbi.nlm.nih.gov/) accession number NP_775184.1, and Uniprot (www.uniprot.org) accession number Q8WWZ1.1. In an embodiment, the IL-38 is a peptide with, or comprising, consisting essentially of or consisting of, an amino acid sequence at least 90%, at least 91%, at least 92%, at least 93%, at least 94%, at least 95%, at least 96%, at least 97%, at least 98%, at least 99% or 100% identical to SEQ ID NO. 49, or comprising, consisting essentially of or consisting of an amino acid sequence encoded by a nucleic acid with a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 50.

In an embodiment the immunomodulatory biomolecule may be a chemokine. The chemokine is preferably selected from chemokine (C-C motif) ligand 1 (CCL1)/TCA3, CCL11, CCL12/MCP-5, CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, C-C motif chemokine ligand 3 like 3 (CCL3L3), CCL4, CCL4L1/LAG-1, CCL5, CCL6, CCL7, CCL8, CCL9, C-X3-C motif chemokine ligand 1 (CX3CL1), C-X-C motif chemokine ligand 1 (CXCL1), CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, IL8/CXCL8, X-C motif chemokine ligand 1 (XCL1), XCL2, FAM19A1, FAM19A2, FAM19A3, FAM19A4 and FAM19A5. In an alternative embodiment the chemokine is selected from a CC chemokine, a CXC chemokine, a C chemokine, a CX₃C chemokine or combinations thereof.

In an embodiment the immunomodulatory biomolecule may be a hormone. Hormones are known to the skilled person to be signaling molecules in multicellular organisms, that are transported to distant organs to regulate physiology and behavior. In an embodiment the hormone is selected from Adrenaline (also known as epinephrine), Melatonin, Noradrenaline (also known as norepinephrine), Triiodothyronine, Thyroxine, Dopamine, Prostaglandins, Leukotrienes, Prostacyclin, Thromboxane, Amylin (also known as Islet Amyloid Polypeptide), Anti-Müllerian hormone (also known as Müllerian-inhibiting factor/hormone), Adiponectin, Adrenocorticotropic hormone (also known as corticotropin), Angiotensinogen, Angiotensin, Antidiuretic hormone (also known as vasopressin, arginine vasopressin), Atrial natriuretic peptide (also known as atriopeptin), Brain natriuretic peptide, Calcitonin, Cholecystokinin, Corticotropin-releasing hormone, Cortistatin, Enkephalin, Endothelin, Erythropoietin, Follicle-stimulating hormone, Galanin, Gastric inhibitory polypeptide, Gastrin, Ghrelin, Glucagon, Glucagon-like peptide-1, Gonadotropin-releasing hormone, Growth hormone-releasing hormone, Hepcidin, Human chorionic gonadotropin, Human placental lactogen, Growth hormone, Inhibin, Insulin, Insulin-like growth factor (also known as somatomedin), Leptin, Lipotropin, Luteinizing hormone, Melanocyte stimulating hormone, Motilin, Orexin, Osteocalcin, Oxytocin (also known as pitocin), Pancreatic polypeptide, Parathyroid hormone, Pituitary adenylate cyclase-activating peptide, Prolactin (also known as leuteotropic hormone), Prolactin-releasing hormone, Relaxin, Renin, Secretin, Somatostatin (also known as growth hormone-inhibiting hormone or growth hormone release-inhibiting hormone or somatotropin release-inhibiting factor or somatotropin release-inhibiting hormone), Thrombopoietin, Thyroid-stimulating hormone (also known as thyrotropin), Thyrotropin-releasing hormone, Vasoactive intestinal peptide, Guanylin or Uroguanylin.

In an embodiment the immunomodulatory biomolecule may be a growth factor. Growth factors are known to the skilled person as naturally occurring substances capable of stimulating cell proliferation, wound healing, and occasionally cellular differentiation. In an embodiment the growth factor is selected from Adrenomedullin (AM), Angiopoietin (Ang), Autocrine motility factor, Bone morphogenetic proteins (BMPs), Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Fibroblast growth factor 1(FGF1), Fibroblast growth factor 2(FGF2), Fibroblast growth factor 3(FGF3), Fibroblast growth factor 4(FGF4), Fibroblast growth factor 5(FGF5), Fibroblast growth factor 6(FGF6), Fibroblast growth factor 7(FGF7), Fibroblast growth factor 8(FGF8), Fibroblast growth factor 9(FGF9), Fibroblast growth factor 10(FGF10), Fibroblast growth factor 11(FGF11), Fibroblast growth factor 12(FGF12), Fibroblast growth factor 13(FGF13), Fibroblast growth factor 14(FGF14), Fibroblast growth factor 15(FGF15), Fibroblast growth factor 16(FGF16), Fibroblast growth factor 17(FGF17), Fibroblast growth factor 18(FGF18), Fibroblast growth factor 19(FGF19), Fibroblast growth factor 20(FGF20), Fibroblast growth factor 21(FGF21), Fibroblast growth factor 22(FGF22), Fibroblast growth factor 23(FGF23), Foetal Bovine Somatotrophin (FBS), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), IL-1, IL-2, IL-3, IL-4, IL-5, IL-6, IL-7, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)-Anti-apoptotic survival factor, T-cell growth factor (TCGF), Thrombopoietin (TPO), Transforming growth factors, Transforming growth factor alpha (TGF-α), Transforming growth factor beta (TGF-β), Tumor necrosis factor-alpha (TNF-α), Vascular endothelial growth factor (VEGF), WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11 and WNT16. In a preferred embodiment the growth factor is selected from VEGF, EGF, CNTF, LIF, Ephrins, FGF, GDNF, HDF, HDGF, IGF, KGF, MSF, NRG, BDNF, NGF, Neurotrophin, PGF, PDGF, RNLS, TCGF, TGF, TNF, WNT or combinations thereof.

In an embodiment the immunomodulatory biomolecule is a hematopoietic growth factor. In an embodiment, the hematopoietic growth factor is selected from IL-3, colony stimulating factor 1 (CSF-1 (M-CSF)), granulocyte-macrophage (GM)-CSF, granulocyte (G)-CSF, a member of the IL-12 family of interleukins or erythropoietin or combinations thereof.

When used herein the term target refers to the object the apolipoprotein (or apolipoprotein mimetic) or fusion protein preferentially binds to. A target may refer to a receptor or cell surface molecule such as a protein or proteoglycan, a cell, a cell type, a tissue or tissue type or an organ.

In particular embodiments, the fusion protein or lipid nanoparticle comprising said fusion protein is capable of binding to a myeloid cell. This may for example be a result of the inherent nature of apolipoproteins, such as ApoA1, to target myeloid cells. When used herein, the terms myeloid cell refers to blood cells that are derived from a progenitor cell for granulocytes, monocytes, erythrocytes, or platelets. Myeloid cells are a major cellular compartment of the immune system comprising monocytes, dendritic cells, tissue macrophages, and granulocytes. The term myeloid compartment, when used herein, refers to the totality of myeloid cells in an organism.

When used herein a rerouting molecule refers to a molecule that allows, when fused to a protein such as an apolipoprotein or apolipoprotein-immunomodulatory biomolecule fusion protein, the protein to bind to a different target than it would bind when the protein was not fused to the rerouting molecule (or in other words, a different target than that to which it would have innately bound), and/or to bind to its intended target with a higher affinity. Binding to a different target may also encompass binding to a particular subset of target cells, including binding to a particular subset of a set of cells which would normally be bound by the apolipoprotein or apolipoprotein-immunomodulatory biomolecule fusion protein. Non limiting examples of rerouting molecules are an antibody or antigen binding fragment thereof or an antibody fragment, a rerouting peptide or a rerouting protein, preferably wherein the rerouting peptide or rerouting protein is a ligand of a receptor present on the target.

It is appreciated that apolipoproteins bind to specific ligands. For example, it is thought that the different apolipoproteins found in different lipoproteins (e.g. HDL, LDL, VLDL, etc.) are responsible for differences in targeting and binding and thus function of the lipoproteins. Without wishing to be bound by theory, it is thought that part of the apolipoprotein has an amphipathic nature and is responsible, together with phospholipids and/or sterols to bind lipids in an aqueous environment, while different parts of the molecule are responsible for interacting with other molecules, e.g. binding to protein receptors. It is further assumed that apolipoproteins may circulate also as proteins, meaning not as lipoproteins. Therefore, the possibility to modify binding affinity of the apolipoprotein (by fusion to a rerouting molecule) provides interesting opportunities, as it allows to fine-tune targeting or binding of the apolipoprotein. Several applications are envisioned for such a fusion protein:

First the rerouting molecule may simply be used to reroute the apolipoprotein or lipoprotein, for example to make changes in the lipid homeostasis. For example, one could envision that LDL or HDL values in the blood of a subject could be altered by using apolipoprotein fusion proteins with a rerouting molecule. This could potentially be exploited for treatment of lipid disorders such as high blood cholesterol levels.

Second it may be used to reroute a lipoprotein (lipid nanoparticle) with a payload to a predetermined target. Lipoproteins or lipid nanoparticles pose interesting methods of carrying a payload such as a pharmaceutical compound. It allows delivery of lipophilic compounds through blood, as the compound may be dissolved in the lipid core of a lipoprotein/lipid nanoparticle. An additional advantage is that the lipoprotein consists of naturally occurring compounds and thus is seen as native by the immune system, avoiding triggering an immune response by the pharmaceutical compound.

Third, it may be combined with an immunomodulatory biomolecule (either as a single fusion protein (apolipoprotein fused to both a rerouting molecule and immunomodulatory biomolecule) or as a combination of two distinct fusion proteins), and as such allow the rerouting of the immunomodulatory biomolecule. As described above, apolipoprotein can essentially serve as a carrier for an immunomodulatory biomolecule. Advantages are that it greatly reduces clearing of the immunomodulatory biomolecule, making it feasible to use immunomodulatory biomolecules that are easily cleared from the blood in a therapeutic setting (e.g. cytokines). One issue that might arise is that an immunomodulatory biomolecule/apolipoprotein fusion protein does not arrive at the intended target for the immunomodulatory biomolecule (i.e. the site, cell, tissue or organ where it is intended to exert its effect). This may be solved by further including a rerouting molecule.

Fourth, the fusion protein comprising the apolipoprotein or apolipoprotein mimetic and rerouting molecule allows to prepare a lipid nanoparticle, wherein the rerouting molecule is exposed to the environment (i.e. aqueous environment) surrounding said apolipoprotein lipid nanoparticle, as a result of its fusion to the apolipoprotein.

Data generated by the inventors suggests that using a rerouting molecule the apolipoprotein fusion protein can successfully be rerouted to a different target. Therefore, in an embodiment, the rerouting molecule is selected from an antibody or an antigen binding fragment thereof, a rerouting peptide or a rerouting protein, preferably wherein the rerouting peptide or rerouting protein is a ligand of a receptor present on the target.

In an embodiment the rerouting molecule may be an antibody or an antigen binding fragment thereof. It is envisioned that any type of antigen binding molecule can in principle be used as a rerouting molecule in the fusion protein according to the invention.

The antibody may be any immunologic binding agent, such as a whole antibody, including without limitation a chimeric, humanized, human, recombinant, transgenic, grafted and single chain antibody, and the like, or any fusion proteins, conjugates, fragments, or derivatives thereof that contain one or more domains that selectively bind to an antigen of interest. The antibody may be a whole immunoglobulin molecule, a monoclonal antibody, a chimeric antibody, a humanized antibody, a human antibody, or an immunologically effective fragment of any of these. Thus, the antibody may encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments.

The terms “specifically bind” or “specifically interact” as used throughout this specification mean that an agent binds to or influences one or more desired molecules or analytes substantially to the exclusion of other molecules which are random or unrelated, and optionally substantially to the exclusion of other molecules that are structurally related. The terms do not necessarily require that an agent binds exclusively to its intended target(s). For example, an agent may be said to specifically bind to target(s) of interest if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, such as, e.g., at least about 1000-fold or more greater, at least about 1×10⁴-fold or more greater, or at least about 1×10⁵-fold or more greater, than its affinity for a non-target molecule.

Therefore, in an embodiment, the antibody or antigen binding fragment thereof is selected from a Fragment antigen-binding region (Fab), a Fab₂, a single-chain variable fragment (scFv), a scFv-Fc, a dAb-Fc, a free light chain antibody, a half antibody, a bispecific Fab2, a Fab₃, a trispecific Fab3 a diabody, a bispecific diabody, a triabody, a trispecific triabody, a minibody, an IgG, an immunoglobulin new antigen receptor (IgNAR), a monovalent IgG, a VₕH or a variable domain of new antigen receptor (VNAR). The antibody or antigen binding fragment may also be a designed antigen binding protein such as but not limited to affibodies, FN3 domains, DARPins or de novo designed protein receptors. It is appreciated that antibodies or antigen binding fragments thereof with a lower molecular weight are preferred due to their reduced size, therefore in a preferred embodiment the antibody or antigen binding fragment thereof is a Fab, scFv, single domain antibody, VₕH or VNAR.

In an embodiment the rerouting molecule may be a rerouting peptide. Non limiting examples of rerouting peptides are receptor binding peptides, ligand mimicking peptides.

Therefore, in an embodiment, the rerouting peptide is selected from programmed cell death protein 1 (PD1) or signal-regulatory protein alpha (SIRPa). It is however understood that any peptide with binding specificity to a cell surface receptor could be used as a rerouting molecule.

In an embodiment the rerouting molecule may be a rerouting protein such as a receptor ligand, a receptor, or interacting protein. Therefore, in an embodiment, the rerouting protein is selected from CD40L or GP120. CD40L can be used to target cells expressing the CD40 receptor. GP120 can be used to bind directly to the CD4 T-Cell co-receptor. It is however understood that any protein with binding specificity to a cell surface receptor could be used as a rerouting molecule.

When used herein, the term lipoprotein refers to a particle, generally a nanoparticle, of at least one apolipoprotein and lipid molecules, dispersed or dissolved in an aqueous environment.

When used herein, the term rerouting refers to targeting the fusion protein to a different target it would normally bind to, or preventing the binding of the regular target of the apolipoprotein or preventing off-target binding. For example, apoA1 is known to bind receptors on myeloid cells, thus the rerouting molecule may be used to bind different cells or prevent binding of myeloid cells. In other words, the rerouting molecule may bind, preferably specifically bind, to non-myeloid cells.

Accordingly, in particular embodiments, the rerouting molecule is capable of binding, preferably specifically binding, to cells that are not myeloid cells, but that may differentiate into myeloid cells, such as a hematopoietic stem and progenitor cell (HSPC), like a hematopoietic stem cell (HSC), a multipotent progenitor (MPP), or a common myeloid progenitor cell (CMP).

In particular embodiments, the rerouting molecule is capable of binding, preferably specifically binding, to a non-myeloid cell, such as a non-myeloid immune cell or an endothelial cell. Endothelial cells may be targeted by use of a rerouting molecule capable of binding to a surface marker of endothelial cells. For example, endothelial cells may be targeted by use of a rerouting molecule capable of binding to Factor VIII-related antigen such as Factor VIII, a rerouting molecule capable of binding to CD31/PECAM-1 such as CD31, a rerouting molecule capable of binding to Angiotensin-converting enzyme (ACE/CD143) such as angiotensin, a rerouting molecule capable of binding to CD34 such as L-selectin or a rerouting molecule capable of binding to endoglin (CD105).

In particular embodiments, the non-myeloid cell is a lymphocyte, such as a T cell, a B cell or a natural killer (NK) cell. Preferably, the lymphocyte is a T cell, even more preferably a CD8+ T cell.

In particular embodiments, if the target cell is a T cell, the rerouting molecule may be an antibody or antigen binding fragment thereof binding, preferably specifically binding, to CD8. For example, the rerouting molecule may be a VHHCD8 as described in Woodham A. W. et al., Nanobody-antigen conjugates elicit HPV-specific antitumor immune responses, Cancer Immunology Research, 2018, Vol. 6, issue 7, and comprising an amino acid sequence as shown in Supplemental Table 1 of said reference.

In particular embodiments, if the target cell is a T and/or B cell, the rerouting molecule or rerouting peptide may be PD1, CD40L or GP120.

The term rerouting may also encompass increasing the specificity of a fusion protein to a particular target, such as a particular target cell. For example, apoA1 is known to bind receptors on myeloid cells, thus the rerouting molecule may be used to bind a particular subtype of myeloid cells.

Accordingly, in particular embodiments, the rerouting molecule is capable of binding, preferably specifically binding, to a myeloid cell selected from the group consisting of megakaryocyte, eosinophil, basophil, erythrocyte, monocyte such as dendritic cell or macrophage, and a neutrophil. For example, SIRPalpha as rerouting molecule may allow to target immunosuppressive macrophages.

It is envisioned the apolipoproteins fusion proteins as described herein may be used as proteins or as lipid nanoparticles. As described above apolipoprotein may circulate as such (meaning not incorporated in a lipoprotein or lipid nanoparticle). This application may be suitable to deliver an immunomodulatory biomolecule, for example a cytokine like IL-4, to a target site. It is known that apolipoproteins can circulate as proteins but may also form lipoproteins in situ. It may however also be advantageous to include the fusion protein in a lipid nanoparticle. Therefore, in an aspect the invention relates to a lipid nanoparticle comprising one or more fusion proteins as herein, the lipid nanoparticle further comprising phospholipids, and optionally sterols. Lipid nanoparticles are conveniently denoted herein as “apolipoprotein lipid nanoparticles”. The fusion protein may be a fusion protein of an apolipoprotein with an immunomodulatory biomolecule, or a fusion protein of an apolipoprotein with a rerouting molecule, or an apolipoprotein with an immunomodulatory biomolecule and a rerouting molecule or combinations thereof. The apolipoprotein may also be an apolipoprotein mimetic. Without being bound to any hypothesis, it is believed that the apolipoprotein or apolipoprotein mimetic may function as a scaffold to help the formation of the nanoparticle together with the phospholipids, and optionally sterols.

In an aspect of the invention, the lipid nanoparticle comprises a fusion protein comprising an apolipoprotein or an apolipoprotein mimetic and an immunomodulatory biomolecule; and phospholipids.

In an aspect of the invention, the lipid nanoparticle comprises a fusion protein comprising an apolipoprotein or an apolipoprotein mimetic and a rerouting molecule; and phospholipids.

The apolipoprotein lipid nanoparticle may comprise both an immunomodulatory biomolecule and a rerouting molecule. This may be achieved by incorporating either one fusion protein comprising both the immunomodulatory biomolecule and the rerouting molecule or by incorporating two fusion proteins, one comprising the immunomodulatory biomolecule and one comprising the rerouting molecule, into the apolipoprotein lipid nanoparticle.

Accordingly, in an aspect of the invention, the lipid nanoparticle comprises a fusion protein comprising an apolipoprotein or an apolipoprotein mimetic, an immunomodulatory biomolecule, and a rerouting molecule; and phospholipids.

In an aspect of the invention, the lipid nanoparticle comprises (i) a fusion protein comprising an apolipoprotein or an apolipoprotein mimetic and an immunomodulatory biomolecule; and phospholipids and (ii) a fusion protein comprising an apolipoprotein or an apolipoprotein mimetic and a rerouting molecule; and phospholipids.

When used herein a lipid nanoparticle (LNP) refers to an assembly of phospholipids and one or more apolipoproteins that is soluble in an aqueous solution. The particles may further comprise sterol and/or lipids (e.g. triglycerides). If the LNP comprises both sterol and lipids, the lipids are encapsulated by the phospholipids and sterols. Accordingly, the lipid nanoparticles thus differ in their structure from liposomes.

In particular embodiments, the phospholipid is selected from a phosphatidylcholine (PC), a phosphatidylethanolamine (PE), a phosphatidylserine and a phosphatidylglycerol or combinations thereof. In further particular embodiments, the phospholipid is selected from the group consisting of 1,2-diphytanoyl-sn-glycero-3-phosphocholine (PHPC), dilauroylphosphatidylcholine (DLPC), dimyristoylphosphatidylcholine (DMPC), dipalmitoylphosphatidylcholine (DPPC), distearoylphosphatidylcholine (DSPC), dioleoylphosphatidylcholine (DOPC), dilauroylphosphatidylglycerol (DLPG), dimyristoylphosphatidylglycerol (DMPG), dipalmitoylphosphatidylglycerol (DPPG), distearoylphosphatidylglycerol (DSPG), dioleoylphosphatidylglycerol (DOPG), dilauroyl phosphatidylethanolamine (DLPE), dimyristoyl phosphatidylethanolamine (DMPE), dipalmitoyl phosphatidylethanolamine (DPPE), distearoyl phosphatidylethanolamine (DSPE), dilauroyl phosphatidylserine (DLPS), dimyristoyl phosphatidylserine (DMPS), dipalmitoyl phosphatidylserine (DPPS), distearoyl phosphatidylserine (DSPS), 1-palmitoyl-2-oleoyl-sn-glycero-3-phosphocholine (POPC), 1,2-dioleoyl-sn-glycero-3-phosphoethanolamine (DOPE), or combinations thereof.

In particular embodiments, the ratio of apolipoprotein (or apolipoprotein mimetic) to phospholipid based on percentage molar weight is between 1:25 and 1:400, more preferably between 1:50 and 1:200, even more preferably between 1:75 and 1:150. In particular embodiments, the ratio of fusion protein as taught herein to phospholipid based on percentage molar weight is between 1:25 and 1:400, more preferably between 1:50 and 1:200, even more preferably between 1:75 and 1:150.

In particular embodiments, the ratio of apolipoprotein (or apolipoprotein mimetic) to phospholipid based on weight is from 3:1 to 1:100. In particular embodiments, the ratio of fusion protein as taught herein to phospholipid based on weight is from 3:1 to 1:100.

In particular embodiments, the sterol is selected from cholesterol, desmosterol, stigmasterol, β-sitosterol, ergosterol, hopanoids, hydroxysteroid, phytosterol, steroids, hydrogenated cholesterol, campesterol, zoosterol, or combinations thereof.

The nanoparticles may comprise further components such as additional proteins or a payload. Therefore, in an embodiment the lipid nanoparticle as defined herein further comprises lipids. In a further embodiment, the lipid nanoparticle as defined herein further comprises a payload.

When used herein the term payload refers to a compound included in a lipid nanoparticle, not being an apolipoprotein, phospholipid or sterol. The payload may for example be a pharmaceutical compound. The lipid nanoparticle is particularly suitable for lipophilic payloads but may also be used for amphipathic molecules. The pharmaceutical compound many be an organic compound, peptide, protein, nucleic acid or nucleic acid analog, biologic or lipid. Therefore, in an embodiment the lipid nanoparticle further comprises a payload, preferably wherein the payload is selected from a nucleic acid or a nucleic acid analog, a therapeutic, a biologic or combinations thereof.

For example, the payload may be a nucleic acid or a nucleic acid analog. Examples may be but are not limited to mRNA, siRNA, miRNA, piRNA, snRNA, snoRNA, srRNA or tsRNA. The nucleic acid analogue may be peptide nucleic acid (PNA), Morpholino and locked nucleic acid (LNA), as well as glycol nucleic acid (GNA), threose nucleic acid (TNA) and hexitol nucleic acids (HNA), or mixtures or combinations thereof. Alternatively, the payload may be a small organic compound such as a small molecule drug. Generally, the small organic compound is synthesized. The therapeutic may for example be an anticancer therapy such as a chemotherapy. Alternatively, the payload may be a biologic. When used herein, the term biologic is used to indicate a biopharmaceutical, also known as a biologic(al) medical product, and can be any pharmaceutical drug product manufactured in, extracted from, or semi-synthesized from biological sources. Biologics can be composed of sugars, proteins, nucleic acids, or complex combinations of these substances, or may be living cells or tissues.

In particular embodiments, the lipid nanoparticle comprises a native (e.g. unfused) apolipoprotein, an apolipoprotein mimetic or a combination thereof, in addition to the apolipoprotein or an apolipoprotein mimetic which forms part of the fusion protein as described herein.

In particular embodiments, the lipid nanoparticle has an average size of 10 to 100 nm, such as from 30 to 100 nm.

In particular embodiments, the lipid nanoparticle is a sphere, a ribbon or a disc, preferably a sphere or a disc, more preferably a sphere.

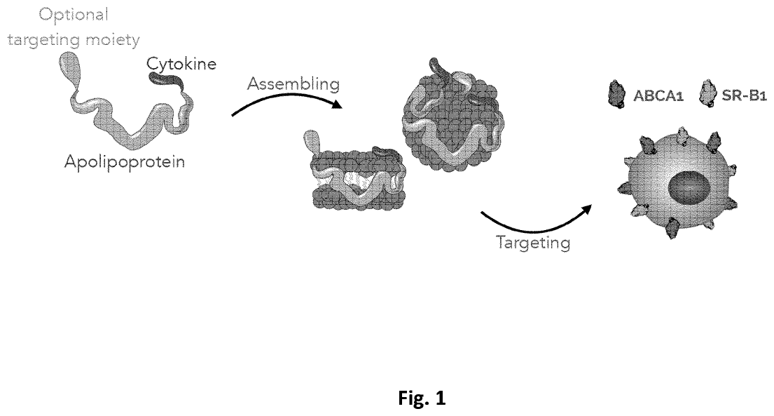

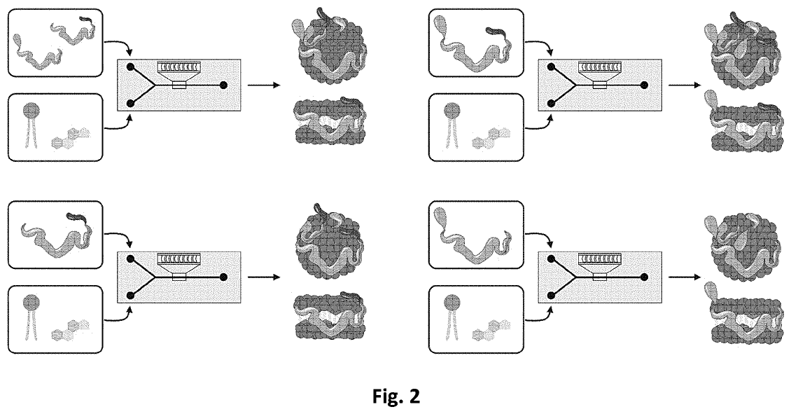

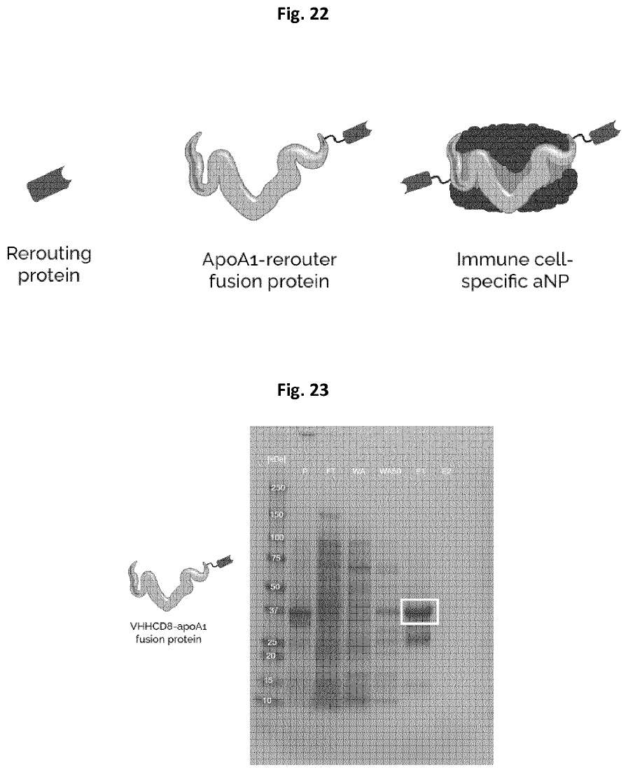

The apolipoprotein or apolipoprotein mimetic forms part of the lipid nanoparticle structure. In particular embodiments, at least a part of said fusion protein is exposed to the environment (i.e. aqueous environment) surrounding said apolipoprotein lipid nanoparticle. Typically, part of the apolipoprotein or apolipoprotein mimetic is exposed to the environment surrounding said apolipoprotein lipid nanoparticle (e.g. see FIG. 1, FIG. 2 and FIG. 22). Furthermore, fusion of an immunomodulatory biomolecule and/or a rerouting molecule to the apolipoprotein or apolipoprotein mimetic typically allows said immunomodulatory biomolecule and/or rerouting molecule to be wholly exposed to the environment surrounding said apolipoprotein lipid nanoparticle (e.g. see FIG. 1, FIG. 2 and FIG. 22). In other words, the immunomodulatory biomolecule and/or a rerouting molecule are not embedded within the lipid nanoparticle. As a result thereof, said immunomodulatory biomolecule and/or said rerouting molecule will be able to move freely and exert its natural function(s), such as its cell targeting function.

In particular embodiments, wherein the lipid nanoparticle comprises a payload, the lipid nanoparticle comprises a core surrounded by a surface layer, wherein the core comprises the payload and the surface layer comprises the apolipoprotein or apolipoprotein mimetic, the phospholipids, the immunomodulatory biomolecule and/or rerouting molecule, and optionally the sterols.

In particular embodiments, the lipid nanoparticle is not a phospholipid bilayer. Also provided herein are methods of manufacturing such lipid nanoparticles. Accordingly, in a further aspect, the invention relates to a method of manufacturing a lipid nanoparticle a method of manufacturing an apolipoprotein lipid nanoparticle as taught herein, the method comprising the steps of:
a1) expressing and isolating one or more apolipoprotein fusion proteins to obtain one or more isolated apolipoprotein fusion proteins,
wherein the one or more apolipoprotein fusion proteins are selected from the group consisting of: an apolipoprotein or apolipoprotein mimetic fused to an immunomodulatory biomolecule, such as IL-4;
an apolipoprotein or apolipoprotein mimetic fused to a rerouting molecule;
an apolipoprotein or apolipoprotein mimetic fused to an immunomodulatory biomolecule, such as IL-4, and a rerouting molecule; and combinations thereof; and/or
a2) chemically conjugating one or more apolipoproteins or apolipoprotein mimetics and isolating the one or more conjugated apolipoproteins to obtain one or more isolated conjugated apolipoproteins,
   wherein the one or more conjugated apolipoproteins are selected from the group consisting of: an apolipoprotein or apolipoprotein mimetic conjugated to an immunomodulatory biomolecule, such as IL-4;
an apolipoprotein or apolipoprotein mimetic conjugated to a rerouting molecule;
an apolipoprotein or apolipoprotein mimetic conjugated to an immunomodulatory biomolecule, such as IL-4, and a rerouting molecule; and combinations thereof; and
b) combining the one or more isolated apolipoprotein fusion proteins obtained in step a1 and/or the one or more isolated conjugated apolipoproteins obtained in step a2 with phospholipids and optionally sterols and/or lipids to obtain an apolipoprotein lipid nanoparticle.

In a further aspect, the invention relates to a method of manufacturing a lipid nanoparticle as defined herein, the method comprising the steps of:
a1) expressing and isolating an apolipoprotein fusion protein to obtain an isolated apolipoprotein fusion protein, where the apolipoprotein fusion protein is an apolipoprotein fused to a cytokine and a targeting moiety and/or wherein the apolipoprotein fusion protein is an apolipoprotein fused to a cytokine and/or an apolipoprotein fused to a targeting moiety; and/or
a2) chemically conjugating an apolipoprotein and isolating the conjugated apolipoprotein to obtain an isolated conjugated apolipoprotein, where the conjugated apolipoprotein is an apolipoprotein conjugated to a cytokine and a targeting moiety and/or wherein the conjugated apolipoprotein is an apolipoprotein conjugated to a cytokine and/or an apolipoprotein conjugated to a targeting moiety;
b) combining the isolated apolipoprotein fusion protein obtained in step a1 and/or the isolated conjugated apolipoprotein obtained in step a2 with phospholipids, and optionally sterols and/or lipids, to obtain a lipid nanoparticle.

In a further aspect, the invention relates to an apolipoprotein lipid nanoparticle obtained by or obtainable by the method of manufacturing an apolipoprotein lipid nanoparticle as taught herein.

It is understood that the fusion protein can be expressed as a chimeric fusion protein of the apolipoprotein with the immunomodulatory biomolecule and/or the rerouting molecule or can be chemically conjugated to the immunomodulatory biomolecule and/or the rerouting molecule or can be produced be a combination of these. Expression of chimeric proteins is known to the skilled person and can be used when the immunomodulatory biomolecule and/or the rerouting molecule is a peptide or protein. It is well within the knowledge of the skilled person to use molecular techniques to produce a nucleic acid encoding such protein, for example by cloning an immunomodulatory biomolecule and/or the rerouting molecule encoding sequence in frame with an apolipoprotein (or mimetic) encoding sequence, for example at C or N terminal sequence encoding nucleotides. An advantage of using chimeric protein expression is that all expressed protein will be fusion protein.

Alternatively chemical conjugation may be used. Suitable methods for chemical conjugation of the immunomodulatory biomolecule and/or the rerouting molecule to the apolipoprotein (or mimetic thereof) are known to the skilled person. Non limiting examples are strain promoted cycloaddition, aminolysis and Michael type addition. For example, an existing or introduced cysteine residue may be used on either the apolipoprotein or the immunomodulatory biomolecule and/or the rerouting molecule.

For example, as described elsewhere herein, the ApoA1 protein may comprise a cysteine in the place of a serine at position 147 or 279. Introduction of a cysteine residue may be achieved by point mutation of a nucleotide in the encoding nucleotide sequence, or by introduction of a cysteine encoding codon. An advantage of chemical conjugation is that it is not limited to the use of peptide or protein sequences but can be applied to any type of organic molecule.

It is understood that the fusion protein, phospholipids, and optional components such as sterols and lipids may be rapidly mixed to obtain a lipid nanoparticle. Optionally added may be lipids and/or a payload as defined herein.

A further aspect of the invention provides a pharmaceutical composition comprising the fusion protein as taught herein, the nucleic acid as taught herein or the lipid nanoparticle as taught herein, and a pharmaceutically acceptable carrier.

In a further aspect the invention relates to fusion protein as defined herein or the lipid nanoparticle as defined herein, or the lipid nanoparticle obtained or obtainable by the method as described herein, or the nucleic acid as defined herein, or the pharmaceutical composition as defined herein for use as a medicament. It is envisioned that either the fusion protein (e.g. by the action of the immunomodulatory biomolecule) or the payload comprised in the nanoparticle may be used to treat, ameliorate or alleviate a symptom in a subject.

In a further aspect, the invention relates to the fusion protein as defined herein or the lipid nanoparticle as defined herein, or the lipid nanoparticle obtained or obtainable by the method as defined herein, or the nucleic acid as defined herein, or the pharmaceutical composition as defined herein for use in the treatment of an immune related disorder. In other words, the invention relates to a method of treating an immune related disorder in a subject in need thereof, comprising administering to said subject a therapeutically effective amount of the fusion protein as defined herein or the lipid nanoparticle as defined herein, or the lipid nanoparticle obtained or obtainable by the method as defined herein, or the nucleic acid as defined herein, or the pharmaceutical composition as defined herein to the subject in need thereof. Also provided herein is the use of the fusion protein as defined herein or the lipid nanoparticle as defined herein, or the lipid nanoparticle obtained or obtainable by the method as defined herein, or the nucleic acid as defined herein, or the pharmaceutical composition as defined herein for the manufacture of a medicament for the treatment of an immune related disorder in a subject.

An immune related disorder as used herein comprises any disorder wherein the immune system plays a role in the disease development. An immune related disorder may refer to a disorder where the immune system is suppressed or where it is (over)activated. Examples of immune related disorders are cancer, infection, sepsis, autoimmune diseases, and cardiovascular diseases. Examples of autoimmune diseases are Type 1 diabetes, Rheumatoid arthritis (RA), Psoriasis/psoriatic arthritis, Multiple sclerosis (MS), Systemic lupus erythematosus (SLE), Inflammatory bowel disease (IBD), Addison's disease, Graves' disease, Sjögren's syndrome, Hashimoto's thyroiditis, Myasthenia gravis, Autoimmune vasculitis, Pernicious anemia, and Celiac disease.

Therefore, in an embodiment the immune related disorder is selected from the group consisting of cancer, infection, sepsis, Type 1 diabetes, Rheumatoid arthritis (RA), Psoriasis/psoriatic arthritis, Multiple sclerosis (MS), Systemic lupus erythematosus (SLE), Inflammatory bowel disease (IBD), Addison's disease, Graves' disease, Sjögren's syndrome, Hashimoto's thyroiditis, Myasthenia gravis, Autoimmune vasculitis, Pernicious anemia, and Celiac disease.

In further particular embodiments, the immune related disorder is selected from the group consisting of cancer, inflammation, an infectious disease, an autoimmune disorder such as multiple sclerosis, allergy, organ transplant rejection, and graft-versus-host disease (GVH).

Unexpectedly, present inventors found that IL-4 can paradoxically reduce inflammation and induce trained immunity simultaneously, particularly when targeted to the myeloid compartment. In many inflammatory problems (such as sepsis, stroke and myocardial infarction), hyperinflammation and immunosuppression concurrently happen. Therefore, the present inventors concluded that fusion proteins of an apolipoprotein or an apolipoprotein mimetic, preferably ApoA1, with IL-4 can be used to prevent immune related disorders by simultaneously reducing inflammation and promoting trained immunity.

Accordingly, in particular embodiments, wherein said immunomodulatory biomolecule is IL-4, the immune related disorder is a disease that benefits from the reduction of inflammation and/or the promotion of trained immunity.

Accordingly, in particular embodiments, wherein said immunomodulatory biomolecule is IL-4, the immune related disorder is a state of hyperinflammation followed by immune paralysis, preferably wherein the hyperinflammation and/or the immune paralysis is caused by an infectious disease such as COVID-19, by sepsis, myocardial infarction, stroke, cancer, or multiple sclerosis.

In particular embodiments, wherein said immunomodulatory biomolecule is IL-4, the apolipoprotein nanoparticle or fusion protein simultaneously reduces inflammation and induces trained immunity.

In particular embodiments, wherein said immunomodulatory biomolecule is IL-2, the apolipoprotein nanoparticle or fusion protein may be used to stimulate T-cell proliferation.

In particular embodiments, wherein said immunomodulatory biomolecule is IL-1p, the apolipoprotein nanoparticle or fusion protein may be used to induce trained immunity.

In particular embodiments, wherein said immunomodulatory biomolecule is IL-38, the apolipoprotein nanoparticle or fusion protein may be used to reduce trained immunity.

In a further aspect, the invention relates to the use of a fusion protein as defined herein or a lipid nanoparticle as defined herein, or a lipid nanoparticle obtained or obtainable by the method as defined herein or the nucleic acid as defined herein, or the pharmaceutical composition as defined herein in delivering a compound or an immunomodulatory molecule to a target, preferably wherein the target is a cell, tissue, and/or organ. In an embodiment the method is an ex vivo or in vitro method. In an alternative embodiment the method is an in vivo method. Typically, the apolipoprotein or rerouting molecule will bind to a cell surface protein, such as a receptor. Therefore, the target may be a protein such as a receptor, a cell or cell type (expressing said protein), a tissue or tissue type (expressing said protein) or an organ (expressing said protein). It is understood that by choosing or adapting the rerouting molecule the fusion protein can be targeted to different proteins. For example, the receptor binding domain of a ligand can be used to target a specific receptor. Alternatively known binding partners of cell surface proteins can be used to reroute the fusion protein.

In a further aspect, the invention relates to a nucleic acid encoding the fusion protein as defined herein. The nucleic acid may be comprised in a vector such as a viral vector for stable integration in a cell, or an expression vector to enable transient expression.

In particular embodiments, the vector comprises a myeloid specific or enhanced promoter or promoter element may be used in a vector to drive myeloid specific expression of IL-4. Suitable promoters are known to the skilled person, non limiting examples are: lysM, Csf1r, CD11c, CX3CR1, Langerin/CD207, MMLV LTR, Visna virus LTR, DC-STAMP, Human MSR, MSR-A, huCD68, CD4, CD2 and Iba-AIF-1 (see e.g. Hume., Journal of leukocyte biology, Volume 89, Issue 4, April 2011, Pages 525-538 for a review). Such promoter or promoter elements can be incorporated in a vector such as for example a lentiviral vector to stable transfect cells and allowing specific expression of the transgene in myeloid cells.

In the inventors' quest to simultaneously resolve hyperinflammation and immune paralysis, they extensively studied the role of interleukin (IL)-4 in trained immunity and tolerance. When investigating its effects on monocytes in vitro [Czimmerer, Z. et al. The Transcription Factor STAT6 Mediates Direct Repression of Inflammatory Enhancers and Limits Activation of Alternatively Polarized Macrophages. Immunity 48, 75-90.e6 (2018); Essner, R., Rhoades, K., McBride, W. H., Morton, D. L. & Economou, J. S. IL-4 down-regulates IL-1 and TNF gene expression in human monocytes. J. Immunol. 142, 3857 (1989); Woodward, E. A., Prêle, C. M., Nicholson, S. E., Kolesnik, T. B. & Hart, P. H. The anti-inflammatory effects of interleukin-4 are not mediated by suppressor of cytokine signalling-1 (SOCS1). Immunology 131, 118-127 (2010)], the inventors discovered that IL-4 simultaneously downregulates inflammatory programs and induces trained immunity. They found that IL-4's unique properties allow overcoming lipopolysaccharide-induced immunoparalysis in monocytes.

However, owing to its unspecific nature and unfavorable pharmacokinetic properties, IL-4 is unsuitable as a myeloid cell-regulating therapeutic. To overcome these limitations, the inventors now found that routing IL-4 to the myeloid compartment is an attractive therapeutic avenue. In support of this concept, the inventors herein describe and developed a fusion protein combining IL-4 with apolipoprotein A-1 (apoA1), the main protein constituent of high-density lipoprotein (HDL), as described elsewhere herein. The resulting apoA1-IL-4 fusion protein readily integrates into myeloid cell-avid lipid nanoparticles (apoA1-IL4-nanoparticles), significantly improving IL-4's pharmacokinetic profile and bioavailability to innate immune cells. The inventors evaluated apoA1-IL-4-nanoparticles' in vivo behavior and safety profile in mice and non-human primates using quantitative nuclear imaging techniques and blood chemistry measurements. Finally, the inventors studied apoA1-IL-4-nanoparticles' therapeutic potential in multiple translational inflammation and sepsis models, institutionalizing a new paradigm for the management of hyperinflammation-induced immunoparalysis.

Therefore the inventors concluded that IL-4 is a promising new therapeutic which can be used to prevent immune related disorders by promoting trained immunity, provided that the IL-4 can be targeted to the myeloid compartment. As described elsewhere herein, the inventors demonstrate this can be achieved though covalent attachment of IL-4 to an apolipoprotein, however it is envisioned that at least the following methods may be used to achieve targeting of the myeloid compartment:
fusion of IL-4 to an apolipoprotein as described elsewhere herein;
fusion of IL-4 to a myeloid-targeting molecule, preferably wherein the myeloid-targeting molecule is an antibody or antigen binding fragment thereof which binds a myeloid marker, or a ligand or peptide that allows targeting of the myeloid compartment;
targeted expression of IL-4 in or near the myeloid compartment.

Accordingly, in a further aspect, the invention relates to a fusion protein of a myeloid-targeting molecule and IL-4, preferably wherein the myeloid-targeting molecule is capable of targeting IL-4 to the myeloid cell.

In particular embodiments, the myeloid-targeting molecule is a chemical substance, such as a small organic molecule, or is a biological molecule, such as a biological polymer, such as for example a protein, polypeptide or peptide, nucleic acid, saccharide, polysaccharide. Preferably, said molecule is a protein, polypeptide or peptide.

As detailed above, the invention is based on the following findings further supported by the experimental examples provided below:
IL-4 surprisingly is able to induce trained immunity, making IL-4 an interesting therapeutic for immune related disorders, particularly for immune paralysis; and
unfavorable pharmacological properties of injected IL-4 can be avoided by targeting IL-4 to the myeloid compartment.

In particular embodiments, the IL-4 is a polypeptide comprising an amino acid sequence at least 90%, at least 91%, at least 92%, at least 93%, at least 94%, at least 95%, at least 96%, at least 97%, at least 98%, at least 99% or 100% identical to SEQ ID NO. 43, or a circular permutation thereof. In a further embodiment the IL-4 polypeptide is encoded be a nucleic acid having a sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 44.

It is understood a fusion with identical function may also be constructed using circular permutation. A circular permutation is a relationship between proteins whereby the proteins have a changed order of amino acids in their peptide sequence. The result is a protein structure with different connectivity, but overall similar three-dimensional shape. For example a first protein has a sequence a-b-c, after the permutation a second protein has a sequence c-a-b while maintaining the same three-dimensional shape. Therefore in a further embodiment the IL-4 polypeptide comprises two sequence which together are at least 90%, at least 91%, at least 92%, at least 93%, at least 94%, at least 95%, at least 96%, at least 97%, at least 98%, at least 99% or 100% identical to SEQ ID NO. 43.

When used herein, the term “targeting”, when referring to targeting a myeloid cell or targeting the myeloid compartment should be understood to mean bring in proximity of the myeloid cell or the myeloid compartment, or to enrich in the proximity of the myeloid cell or the myeloid compartment. This implies that when targeting (by the myeloid-targeting molecule) on average more molecules of IL-4 are in proximity of a myeloid cell or the myeloid compartment. In proximity herein means being located such that the IL-4 can interact with the myeloid cell, e.g. by binding of one of its receptors.

When used herein, a a myeloid-targeting molecule is intended to indicate any molecule, but preferably a peptide, protein or part of a protein such as a protein domain, that, when fused to IL-4 allows IL-4 to be targeted to a myeloid cell. As explained in more detail below, the following options are demonstrated or envisioned to serve as myeloid-targeting molecules: apolipoproteins, antibodies or antigen binding fragments thereof, myeloid cell specific ligands of receptors or membrane molecules.

When used herein, the terms myeloid cell refers to blood cells that are derived from a progenitor cell for granulocytes, monocytes, erythrocytes, or platelets. Myeloid cells are a major cellular compartment of the immune system comprising monocytes, dendritic cells, tissue macrophages, and granulocytes. The term myeloid compartment, when used herein, refers to the totality of myeloid cells in an organism.

In particular embodiments, the myeloid-targeting molecule is selected from an antibody or an antigen binding fragment thereof, a myeloid-targeting peptide or a myeloid-targeting protein, preferably wherein the myeloid-targeting peptide or myeloid-targeting protein is a ligand of a receptor present on the target.

For example, an antibody or antigen binding fragment thereof can be used that specifically binds to an antigen which is highly expressed or exclusively present on myeloid cells. Suitable targets are known to the skilled person, non-limiting examples are CD11b, CD11c, CD14 or co-stimulatory molecules such as CD80, CD83, CD86, CD40 or HLA-DR. Therefore in an embodiment the myeloid-targeting peptide or protein is selected from an antibody or an antigen binding fragment thereof which selectively binds to CD11b, CD11c, CD14, CD80, CD83, CD86, CD40 or HLA-DR.

In particular embodiments, the antibody or antigen binding fragment thereof is selected from a Fab, a Fab2, a scFv, a scFv-Fc, a dAb-Fc, a free light chain antibody, a half antibody, a bispecific Fab¬2¬, a Fab3, a trispecific Fab¬3¬ a diabody, a bispecific diabody, a triabody, a trispecific triabody, a minibody, an IgG, an IgNAR, a monovalent IgG, a VhH or a VNAR.

Alternatively a ligand or cofactor may be used which specifically or predominantly binds to a receptor or factor expressed on a myeloid cell, non-limiting examples being CD40L (CD154) and FC domains, but the skilled person is aware of other suitable ligands or cofactors. Therefore, in an embodiment the a myeloid-targeting molecule is a myeloid-targeting peptide or a myeloid-targeting protein wherein the myeloid-targeting protein or myeloid-targeting peptide is selected from CD40L (CD154) and FC domains.

In a further aspect, the invention relates to a nucleic acid encoding the fusion protein of a myeloid-targeting molecule and IL-4 as taught herein. In an embodiment the invention relates to a nucleic acid having a nucleic acid sequence at least 70%, 75%, 80%, 85%, 90%, 91%, 92%, 93%, 94%, 95%, 96%, 97%, 98%, 99% or 100% identical to SEQ ID NO. 44 or comprising a nucleic acid sequence encoding a polypeptide having a sequence at least 90%, at least 91%, at least 92%, at least 93%, at least 94%, at least 95%, at least 96%, at least 97%, at least 98%, at least 99% or 100% identical to SEQ ID NO. 43.

The nucleic acid sequence can be used to express the fusion protein according to the invention, therefore in a preferred embodiment the nucleic acid is or is comprised in a vector, such as a protein expression vector or viral vector. The fusion protein can be expressed ex vivo, e.g. to be administrated to a subject later. Alternatively, the vector may be used to transiently or stably transform a cell in the subject. For example it may be particularly beneficial to transform hepatocytes, fibroblasts or myocytes, allowing them to express the fusion protein which can subsequently be released in the blood circulation to allow targeting of the myeloid compartment. When used herein, the term vector refers to a plasmid or virus designed for gene expression in cells. The vector is used to introduce a specific gene into a target cell, and can commandeer the cell's mechanism for protein synthesis to produce the protein encoded by the gene. The vector is engineered to contain regulatory sequences that act as enhancer and promoter regions and lead to efficient transcription of the gene carried on the expression vector. The skilled person is aware how to adapt the enhancer and promoter regions for example for cell type specific or inducible expression of the gene (and subsequent translation into a protein).

It is further envisioned that instead of expressing an IL-4 fusion protein with a myeloid-targeting molecule which targets a myeloid cell, IL-4 can be expressed in or near a myeloid cell to ensure targeting of the IL-4 to the myeloid cell. Therefore, a further aspect provides a nucleic acid comprising a nucleic acid sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 44 or comprising a nucleic acid sequence encoding a polypeptide having a sequence at least 90%, at least 91%, at least 92%, at least 93%, at least 94%, at least 95%, at least 96%, at least 97%, at least 98%, at least 99% or 100% identical to SEQ ID NO. 43 and further comprising means for targeted expression in a myeloid cell, wherein said mean are selected from:
a promoter for selective or inducible expression in said myeloid cell operatively linked to said nucleic acid; or
a viral expression vector comprising said nucleic acid capable of stably expressing said nucleic acid in said myeloid cell; or
a lipid nanoparticle comprising one or more apolipoproteins, phospholipids, said nucleic acid and optionally sterol.

For example, a myeloid specific or enhanced promoter or promoter element may be used in a vector to drive myeloid specific expression of IL-4. Suitable promoters are known to the skilled person, non limiting examples are: lysM, Csf1r, CD11c, CX3CR1, Langerin/CD207, MMLV LTR, Visna virus LTR, DC-STAMP, Human MSR, MSR-A, huCD68, CD4, CD2 and Iba-AIF-1 (see e.g. Hume., Journal of leukocyte biology, Volume89, Issue4, April 2011, Pages 525-538 for a review). Such promoter or promoter elements can be incorporated in a vector such as for example a lentiviral vector to stable transfect cells and allowing specific expression of the transgene in myeloid cells.

For example, viruses such as modified retroviruses may be used to specifically infect and drive expression of IL-4 in myeloid cells.

For example, lipid nanoparticles comprising apolipoproteins, cholesterol and phospholipids comprising mRNA encoding IL-4 may be used to specifically target myeloid cells and translate the mRNA into protein in the myeloid cells.

The inventors have demonstrated that IL-4 can reduce inflammation and induce trained immunity, particularly when targeted to the myeloid compartment. Therefore, a further aspect provides the fusion protein of a myeloid-targeting molecule and IL-4 or a nucleic acid encoding said fusion protein for use as a medicament. A further aspect provides the fusion protein of a myeloid-targeting peptide or protein and IL-4 or a nucleic acid encoding said fusion protein for use in the treatment of an immune related disorder, preferably wherein the immune related disorder is a state of hyperinflammation followed by immune paralysis, preferably wherein the hyperinflammation and/or the immune paralysis is caused by an infectious disease such as COVID-19, by sepsis, myocardial infarction or stroke.

In other words, a further aspect provides a method of treating a subject in need thereof comprising administering the fusion protein of a myeloid-targeting peptide or protein and IL-4 or a nucleic acid encoding said fusion protein, preferably wherein the method of treatment is a method of treating an immune-related disorder.

The present inventors have shown for the first time that IL-4 can induce trained immunity. Further the inventors demonstrate that the unfavorable pharmacological properties of IL-4 (e.g. the extreme short half-life) can be avoided by targeting myeloid cells in an organism. Experimental data detailed below demonstrates the use of IL-4 as a targeted therapeutic in cases of hyperinflammation followed by immune paralysis, such as is the case in infectious disease such as COVID-19, by sepsis, myocardial infarction or stroke.

Accordingly, in particular embodiments, the fusion protein or nucleic acid for use as taught herein comprises reducing inflammation and/or stimulating or promoting trained immunity.

A further aspect provides in vivo, in vitro or ex vivo use of IL-4 in stimulating or promoting trained immunity in a cell, organ, tissue or an organism.

It will be appreciated by persons skilled in the art that numerous variations and/or modifications may be made to the above-described aspects and/or embodiments, without departing from the broad general scope of the present invention. The present aspects and/or embodiments are, therefore, to be considered in all respects as illustrative and not restrictive. The present invention includes the following non-limiting examples.

The present application also provides aspects and embodiments as set forth in the following Statements*:
Statement 1*. A fusion protein of an apolipoprotein or an apolipoprotein mimetic with an immunomodulatory biomolecule, wherein the immunomodulatory biomolecule is a protein that enhances or suppresses an immune response.
Statement 2*.A fusion protein of an apolipoprotein or an apolipoprotein mimetic with a rerouting molecule, wherein the rerouting molecule is a molecule that allows that fusion protein to bind to a different target than to which the apolipoprotein or apolipoprotein mimetic would have bound (or in other words, wherein the rerouting molecule is a molecule that allows, when fused to the apolipoprotein, the apolipoprotein to bind to a different target than it would bind when the apolipoprotein was not fused to the rerouting molecule).
Statement 3*.A fusion protein of an apolipoprotein or an apolipoprotein mimetic with an immunomodulatory biomolecule and a rerouting molecule.
Statement 4*.The fusion protein according to any one of the previous statements*, wherein the immunomodulatory biomolecule is selected from a cytokine, a chemokine, a hormone, a growth factor, a hematopoietic growth factor or combinations thereof.
Statement 5*.The fusion protein according to statement 4*, wherein the cytokine is selected from the IL-2 subfamily, the interferon subfamily, the IL-10 subfamily, the IL-1 family, the TGFbeta family, or the IL-17 family, or combinations thereof, more preferably wherein the cytokine is selected from IL-1P, IL-2, IL-4, IL-38, or combinations thereof; and/or
   wherein the chemokine is selected from a CC chemokine, a CXC chemokine, a C chemokine, a CX₃C chemokine or combinations thereof; and/or
   wherein the growth factor is selected from VEGF, EGF, CNTF, LIF, Ephrins, FGF, GDNF, HDF, HDGF, IGF, KGF, MSF, NRG, BDNF, NGF, Neurotrophin, PGF, PDGF, RNLS, TCGF, TGF, TNF and WNT or combinations thereof; and/or
   wherein the hematopoietic growth factor is selected from IL-3, CSF-1 (M-CSF), GM-CSF, G-CSF, a member of the IL-12 family of interleukins or erythropoietin or combinations thereof.
Statement 6*.The fusion protein according to any one of the previous statements*, wherein the rerouting molecule is selected from an antibody or an antigen binding fragment thereof, a rerouting peptide or a rerouting protein, preferably wherein the rerouting peptide or rerouting protein is a ligand of a receptor present on the target.
Statement 7*.The fusion protein according to statement 6*, wherein the antibody or antigen binding fragment thereof is selected from a Fab, a Fab₂, a scFv, a scFv-Fc, a dAb-Fc, a free light chain antibody, a half antibody, a bispecific Fab2, a Fab₃, a trispecific Fab3 a diabody, a bispecific diabody, a triabody, a trispecific triabody, a minibody, an IgG, an IgNAR, a monovalent IgG, a VhH or a VNAR; and/or
   wherein the rerouting peptide is selected from PD1 or SIRPa; and/or
   wherein the rerouting protein is selected from CD40L or GP120.
Statement 8*.The fusion protein according to any one of the previous statements*, wherein the apolipoprotein or apolipoprotein mimetic is an ApoA1, ApoA4, ApoC3, ApoD, ApoE, ApoL1, ApoL3 or a mimetic thereof.
Statement 9*. A lipid nanoparticle comprising one or more fusion proteins as defined in statement 1* or 4* or 5* or 8* and/or one or more fusion protein as defined in statement 2* or 6* or 7* or 8* and/or one or more fusion protein as defined in statements 3* to 8*, the lipid nanoparticle further comprising phospholipids, and sterols.
Statement 10*. Lipid nanoparticle as defined in statement 9*, wherein the lipid nanoparticle further comprises lipids.
Statement 11*. Lipid nanoparticle according to statement 9* or 10*, wherein the lipid nanoparticle further comprises a payload, preferably wherein the payload is selected from a nucleic acid or a nucleic acid analog, a therapeutic, a biologic or combinations thereof.
Statement 12*. Method of manufacturing a lipid nanoparticle as defined in any one of statements 9* to 11*, the method comprising the steps of:
a1) expressing and isolating an apolipoprotein fusion protein to obtain an isolated apolipoprotein fusion protein, where the apolipoprotein fusion protein is an apolipoprotein fused to a cytokine and a targeting moiety and/or wherein the apolipoprotein fusion protein is an apolipoprotein fused to a cytokine and/or an apolipoprotein fused to a targeting moiety; and/or
a2) chemically conjugating an apolipoprotein and isolating the conjugated apolipoprotein to obtain an isolated conjugated apolipoprotein, where the conjugated apolipoprotein is an apolipoprotein conjugated to a cytokine and a targeting moiety and/or wherein the conjugated apolipoprotein is an apolipoprotein conjugated to a cytokine and/or an apolipoprotein conjugated to a targeting moiety;
b) combining the isolated apolipoprotein fusion protein obtained in step a1 and/or the isolated conjugated apolipoprotein obtained in step a2 with phospholipids, sterols and optionally lipids to obtain a lipid nanoparticle.
Statement 13*. The fusion protein according to any one of statements 1* to 8* or the lipid nanoparticle according to any one of statements 9* to 11*, or the lipid nanoparticle obtained or obtainable by the method of statement 12* for use as a medicament.
Statement 14*. The fusion protein according to any one of statements 1* to 8* or the lipid nanoparticle according to any one of statements 9* to 11*, or the lipid nanoparticle obtained or obtainable by the method of statement 12* for use in the treatment of an immune related disorder.
Statement 15*. Use of a fusion protein according to any one of statements 1* to 8* or a lipid nanoparticle according to any one of statements 9* to 11*, or a lipid nanoparticle obtained or obtainable by the method of statement 12* in delivering a compound to a target, preferably wherein the target is a cell, tissue, and/or organ.
Statement 16*. A nucleic acid encoding the fusion protein as defined in any one of statements 1* to 8*.

The present application also provides aspects and embodiments as set forth in the following Statements:
Statement 1. An apolipoprotein lipid nanoparticle comprising
   a fusion protein comprising an apolipoprotein or an apolipoprotein mimetic and an immunomodulatory biomolecule; and
   phospholipids;
wherein the immunomodulatory biomolecule is a protein that enhances or suppresses an immune response.
Statement 2. An apolipoprotein lipid nanoparticle comprising
   a fusion protein comprising an apolipoprotein or an apolipoprotein mimetic and a rerouting molecule; and
   phospholipids;
wherein the rerouting molecule is a molecule that allows that fusion protein to bind to a different target than to which the apolipoprotein or apolipoprotein mimetic would have bound and/or to bind to its intended target with a higher affinity.
Statement 3. An apolipoprotein lipid nanoparticle comprising
   a fusion protein comprising an apolipoprotein or an apolipoprotein mimetic, an immunomodulatory biomolecule, and a rerouting molecule; and
   phospholipids;
wherein the immunomodulatory biomolecule is a protein that enhances or suppresses an immune response; and
wherein the rerouting molecule is a molecule that allows that fusion protein to bind to a different target than to which the apolipoprotein or apolipoprotein mimetic would have bound and/or to bind to its intended target with a higher affinity.
Statement 4. An apolipoprotein lipid nanoparticle comprising
   the fusion protein as defined in statement 1;
   the fusion protein as defined in statement 2; and
   phospholipids.
Statement 5. The apolipoprotein lipid nanoparticle according to any one of statements 1 to 4, wherein the apolipoprotein lipid nanoparticle further comprises sterols.
Statement 6. The apolipoprotein lipid nanoparticle according to any one of statements 1 to 5, wherein the apolipoprotein lipid nanoparticle further comprises lipids, preferably triglycerides.
Statement 7. The apolipoprotein lipid nanoparticle according to any one of statements 1 to 6, wherein said apolipoprotein lipid nanoparticle is a sphere, a ribbon or a disc.
Statement 8. The apolipoprotein lipid nanoparticle according to any one of statements 1 to 7, wherein at least a part of said fusion protein is exposed to the environment surrounding said apolipoprotein lipid nanoparticle, preferably wherein said immunomodulatory biomolecule and/or said rerouting molecule is exposed to the environment surrounding said apolipoprotein lipid nanoparticle.
Statement 9. The apolipoprotein lipid nanoparticle according to any one of statements 1 or 3 to 8, wherein the immunomodulatory biomolecule is selected from the group consisting of a cytokine, a chemokine, a hormone, a growth factor, a hematopoietic growth factor, and combinations thereof.
Statement 10. The apolipoprotein lipid nanoparticle according to statement 9, wherein the cytokine is selected from the group consisting of the IL-2 subfamily, the interferon subfamily, the IL-10 subfamily, the IL-1 family, the TGFbeta family, or the IL-17 family, and combinations thereof, more preferably wherein the cytokine is selected from the group consisting of IL-1p, IL-2, IL-4, IL-38, and combinations thereof; and/or
   wherein the chemokine is selected from the group consisting of a CC chemokine, a CXC chemokine, a C chemokine, a CX₃C chemokine, and combinations thereof; and/or
   wherein the growth factor is selected from the group consisting of VEGF, EGF, CNTF, LIF, Ephrins, FGF, GDNF, HDF, HDGF, IGF, KGF, MSF, NRG, BDNF, NGF, Neurotrophin, PGF, PDGF, RNLS, TCGF, TGF, TNF and WNT, and combinations thereof; and/or wherein the hematopoietic growth factor is selected from the group consisting of IL-3, CSF-1 (M-CSF), GM-CSF, G-CSF, a member of the IL-12 family of interleukins or erythropoietin, and combinations thereof.
Statement 11. The apolipoprotein lipid nanoparticle according to statement 9 or 10, wherein the cytokine is IL-4.
Statement 12. The apolipoprotein lipid nanoparticle according to any one of statements 2 to 11, wherein the rerouting molecule is selected from an antibody or an antigen binding fragment thereof, a rerouting peptide or a rerouting protein, preferably wherein the rerouting peptide or rerouting protein is a ligand of a receptor present on the target.
Statement 13. The apolipoprotein lipid nanoparticle according to statement 12, wherein the antibody or antigen binding fragment thereof is selected from the group consisting of a Fab, a Fab₂, a scFv, a scFv-Fc, a dAb-Fc, a free light chain antibody, a half antibody, a bispecific Fab2, a Fab₃, a trispecific Fab3 a diabody, a bispecific diabody, a triabody, a trispecific triabody, a minibody, an IgG, an IgNAR, a monovalent IgG, a VhH, and a variable new antigen receptor (VNAR).
Statement 14. The apolipoprotein lipid nanoparticle according to any one of statements 2 to 13, wherein the rerouting molecule is capable of binding to a hematopoietic stem and progenitor cell (HSPC), such as a hematopoietic stem cell (HSC), a multipotent progenitor (MPP), or a common myeloid progenitor cell (CMP).
Statement 15. The apolipoprotein lipid nanoparticle according to any one of statements 2 to 13, wherein the rerouting molecule is capable of binding to a myeloid cell selected from the group consisting of megakaryocyte, eosinophil, basophil, erythrocyte, monocyte such as dendritic cell or macrophage, and a neutrophil.
Statement 16. The apolipoprotein lipid nanoparticle according to statement 15, wherein the rerouting peptide is SIRPa.
Statement 17. The apolipoprotein lipid nanoparticle according to any one of statements 2 to 13, wherein the rerouting molecule is capable of binding to a non-myeloid cell, such as a non-myeloid immune cell or an endothelial cell.
Statement 18. The apolipoprotein lipid nanoparticle according to statement 17, wherein the rerouting molecule is capable of binding to lymphocytes, preferably T cells, more preferably CD8+ T cells.
Statement 19. The apolipoprotein lipid nanoparticle according to statement 17, wherein the rerouting molecule is an antibody or antigen binding fragment thereof specifically binding to CD8 or wherein the rerouting peptide is PD1, CD40L or GP120.
Statement 20. The apolipoprotein lipid nanoparticle according to any one of statements 1 to 19, wherein the apolipoprotein is an ApoA1, ApoA-1 Milano, ApoA4, ApoC3, ApoD, ApoE, ApoL1, ApoL3 or the apolipoprotein mimetic is a mimetic of an ApoA1, ApoA-1 Milano, ApoA4, ApoC3, ApoD, ApoE, ApoL1, ApoL3.
Statement 21. The apolipoprotein lipid nanoparticle according to any one of statements 1 to 20, wherein the apolipoprotein lipid nanoparticle comprises a payload, preferably wherein the payload is selected from a nucleic acid or a nucleic acid analog, a therapeutic, a biologic or combinations thereof.
Statement 22. Method of manufacturing an apolipoprotein lipid nanoparticle as defined in any one of statements 1 to 21, the method comprising the steps of:
a1) expressing and isolating one or more apolipoprotein fusion proteins to obtain one or more isolated apolipoprotein fusion proteins,
   wherein the one or more apolipoprotein fusion proteins are selected from the group consisting of: an apolipoprotein or apolipoprotein mimetic fused to an immunomodulatory biomolecule;
   an apolipoprotein or apolipoprotein mimetic fused to a rerouting molecule;
   an apolipoprotein or apolipoprotein mimetic fused to an immunomodulatory biomolecule and a rerouting molecule; and combinations thereof; and/or
a2) chemically conjugating one or more apolipoproteins or apolipoprotein mimetics and isolating the one or more conjugated apolipoproteins to obtain one or more isolated conjugated apolipoproteins,
   wherein the one or more conjugated apolipoproteins are selected from the group consisting of: an apolipoprotein or apolipoprotein mimetic conjugated to an immunomodulatory biomolecule;
   an apolipoprotein or apolipoprotein mimetic conjugated to a rerouting molecule;
   an apolipoprotein or apolipoprotein mimetic conjugated to an immunomodulatory biomolecule and a rerouting molecule; and combinations thereof; and
b) combining the one or more isolated apolipoprotein fusion proteins obtained in step a1 and/or the one or more isolated conjugated apolipoproteins obtained in step a2 with phospholipids and optionally sterols and/or lipids to obtain an apolipoprotein lipid nanoparticle.
Statement 23. An apolipoprotein lipid nanoparticle obtained by or obtainable by the method of statement 22.
Statement 24. A pharmaceutical composition comprising the apolipoprotein lipid nanoparticle according to any one of statements 1 to 21 or 23, and a pharmaceutically acceptable carrier.
Statement 25. The apolipoprotein lipid nanoparticle according to any one of statements 1 to 21 or 23 or the pharmaceutical composition according to statement 24 for use as a medicament.
Statement 26. The apolipoprotein lipid nanoparticle according to any one of statements 1 to 21 or 23 or the pharmaceutical composition according to statement 24 for use in the treatment of an immune related disorder.
Statement 27. The apolipoprotein lipid nanoparticle for use according to statement 26 or the pharmaceutical composition for use according to statement 26, wherein the immune related disorder is selected from the group consisting of cancer, inflammation, an infectious disease, an autoimmune disorder, allergy, organ transplant rejection, and graft-versus-host disease (GVH).
Statement 28. The apolipoprotein lipid nanoparticle for use according to statement 26 or the pharmaceutical composition for use according to statement 26, wherein the immunomodulatory biomolecule is IL-4 and wherein the immune related disorder is a state of hyperinflammation followed by immune paralysis, preferably wherein the hyperinflammation and/or the immune paralysis is caused by an infectious disease such as COVID-19, by sepsis, myocardial infarction, stroke, cancer, or multiple sclerosis.
Statement 29. The apolipoprotein lipid nanoparticle according to any one of statements 1, 3 to 21 or 23 or the pharmaceutical composition according to statement 24 for use in targeting said immunomodulatory biomolecule to a target cell.
Statement 30. The apolipoprotein lipid nanoparticle according to any one of statements 1, 3 to 13, 15, 16, 20, 21 or 23 or the pharmaceutical composition according to statement 24 for use in targeting said immunomodulatory biomolecule to a myeloid cell.
Statement 31. Use of the apolipoprotein lipid nanoparticle according to any one of statements 1, 3 to 21 or 23 for delivering an immunomodulatory biomolecule to a target, preferably wherein the target is a cell, tissue, and/or organ.
Statement 32. A fusion protein comprising an apolipoprotein or an apolipoprotein mimetic and an immunomodulatory biomolecule, wherein the immunomodulatory biomolecule is a protein that enhances or suppresses an immune response, for use in targeting said immunomodulatory biomolecule to a myeloid cell.
Statement 33. The fusion protein for use according to statement 32, wherein the fusion protein further comprises a rerouting molecule, wherein the rerouting molecule is a molecule that allows that fusion protein to bind to a different target than to which the apolipoprotein or apolipoprotein mimetic would have bound and/or to bind to its intended target with a higher affinity, preferably wherein the rerouting molecule is a rerouting molecule as defined in statement 15.
Statement 34. A fusion protein comprising an apolipoprotein or an apolipoprotein mimetic and a rerouting molecule, wherein the rerouting molecule is a molecule that allows that fusion protein to bind to a different target than to which the apolipoprotein or apolipoprotein mimetic would have bound and/or to bind to its intended target with a higher affinity.
Statement 35. The fusion protein according to statement 34, wherein the rerouting molecule is a rerouting molecule as defined in any one of statements 12 to 19.
Statement 36. The fusion protein according to statement 34 or 35, wherein the fusion protein further comprises an immunomodulatory biomolecule, wherein the immunomodulatory biomolecule is a protein that enhances or suppresses an immune response, preferably wherein the immunomodulatory biomolecule is an immunomodulatory biomolecule as defined in statement 9 or 10.
Statement 37. A nucleic acid encoding the fusion protein according to any one of statements 34 to 36.
Statement 38. A pharmaceutical composition comprising the fusion protein according to any one of statements 34 to 36 or the nucleic acid according to statement 37, and a pharmaceutically acceptable carrier.
Statement 39. The fusion protein according to any one of statements 34 to 36, the nucleic acid according to statement 37 or the pharmaceutical composition according to statement 38 for use as a medicament.
Statement 40. The fusion protein according to any one of statements 34 to 36, the nucleic acid according to statement 37 or the pharmaceutical composition according to statement 38 for use in the treatment of an immune related disorder, preferably wherein the immune related disorder is an immune related disorder selected from the group consisting of cancer, inflammation, an infectious disease, an autoimmune disorder, allergy, organ transplant rejection, and graft-versus-host disease (GVH).
Statement 41. The fusion protein according to statement 36, the nucleic acid encoding the fusion protein according to statement 36 or the pharmaceutical composition according to statement 38 when being dependent from statement 36 for use in targeting said immunomodulatory biomolecule to a target cell.
Statement 42. A fusion protein comprising an apolipoprotein or an apolipoprotein mimetic and interleukin-4 (IL-4).
Statement 43. The fusion protein according to statement 42, wherein the fusion protein further comprises a rerouting molecule, wherein the rerouting molecule is a molecule that allows that fusion protein to bind to a different target than to which the apolipoprotein or apolipoprotein mimetic would have bound and/or to bind to its intended target with a higher affinity, preferably wherein the rerouting molecule is a rerouting molecule as defined in any one of statements 12 to 19.
Statement 44. The fusion protein according to statement 42 or 43, wherein the apolipoprotein or apolipoprotein mimetic is as defined in statement 20.
Statement 45. A nucleic acid encoding the fusion protein according to any one of statements 42 to 44.
Statement 46. A pharmaceutical composition comprising the fusion protein according to any one of statements 42 to 44 or the nucleic acid according to statement 45, and a pharmaceutically acceptable carrier.
Statement 47. The fusion protein according to any one of statements 42 to 44, the nucleic acid according to statement 45, or the pharmaceutical composition according to statement 46 for use as a medicament.
Statement 48. The fusion protein according to any one of statements 42 to 44, the nucleic acid according to statement 45, or the pharmaceutical composition according to statement 46 for use in the treatment of an immune related disorder.
Statement 49. The fusion protein for use according to statement 48, the nucleic acid for use according to statement 48, or the pharmaceutical composition for use according to statement 48, wherein the immune related disorder is a state of hyperinflammation followed by immune paralysis, preferably wherein the hyperinflammation and/or the immune paralysis is caused by an infectious disease such as COVID-19, by sepsis, myocardial infarction, stroke, cancer, or multiple sclerosis.
Statement 50. The fusion protein according to any one of statements 42 to 44, the nucleic acid according to statement 45, or the pharmaceutical composition according to statement 46 for use in targeting IL-4 to a target cell.
Statement 51. The fusion protein according to any one of statements 42 to 44, the nucleic acid according to statement 45, or the pharmaceutical composition according to statement 46 for use in targeting IL-4 to a myeloid cell.
Statement 52. A fusion protein comprising a myeloid-targeting molecule and IL-4, wherein the myeloid-targeting molecule is capable of targeting the IL-4 to a myeloid cell.
Statement 53. The fusion protein according to statement 52, wherein the IL-4 is a polypeptide comprising an amino acid sequence at least 90%, at least 91%, at least 92%, at least 93%, at least 94%, at least 95%, at least 96%, at least 97%, at least 98%, at least 99% or 100% identical to SEQ ID NO. 43, or a circular permutation thereof.
Statement 54. The fusion protein according to statement 52 or 53, wherein the myeloid-targeting molecule is selected from an antibody or an antigen binding fragment thereof, a myeloid-targeting peptide or a myeloid-targeting protein, preferably wherein the myeloid-targeting peptide or myeloid-targeting protein is a ligand of a receptor present on the target.
Statement 55. The fusion protein according to statement 54, wherein the antibody or antigen binding fragment thereof is selected from a Fab, a Fab2, a scFv, a scFv-Fc, a dAb-Fc, a free light chain antibody, a half antibody, a bispecific Fab¬2¬, a Fab3, a trispecific Fab¬3¬ a diabody, a bispecific diabody, a triabody, a trispecific triabody, a minibody, an IgG, an IgNAR, a monovalent IgG, a VhH or a VNAR.
Statement 56. A nucleic acid encoding the fusion protein according to any one of statements 52 to 55.
Statement 57. A nucleic acid comprising a nucleic acid sequence at least 70%, at least 75%, at least 80%, at least 85%, at least 90%, at least 91%, at least 92%, at least 93%, at least 94%, at least 95%, at least 96%, at least 97%, at least 98%, at least 99% or 100% identical to SEQ ID NO. 44 or comprising a nucleic acid sequence encoding a polypeptide having a sequence at least 90%, at least 91%, at least 92%, at least 93%, at least 94%, at least 95%, at least 96%, at least 97%, at least 98%, at least 99% or 100% identical to SEQ ID NO. 43 and further comprising means for targeted expression in a myeloid cell, wherein said mean are selected from:
   a promoter for selective or inducible expression in said myeloid cell operatively linked to said nucleic acid; or
   a viral expression vector comprising said nucleic acid capable of stably expressing said nucleic acid in said myeloid cell; or
   a lipid nanoparticle comprising one or more apolipoproteins, phospholipids, said nucleic acid, and optionally sterol.
Statement 58. A pharmaceutical composition comprising the fusion protein according to any one of statements 52 to 55 or the nucleic acid according to statement 56 or 57, and a pharmaceutically acceptable carrier.
Statement 59. The fusion protein according to any one of statements 52 to 55, the nucleic acid according to statement 56 or 57, or the pharmaceutical composition according to statement 58 for use as a medicament.
Statement 60. The fusion protein according to any one of statements 52 to 55, the nucleic acid according to statement 56 or 57, or the pharmaceutical composition according to statement 58 for use in the treatment of an immune related disorder.
Statement 61. The fusion protein for use according to statement 60, the nucleic acid for use according to statement 60, or the pharmaceutical composition for use according to statement 60, wherein the immune related disorder is a state of hyperinflammation followed by immune paralysis, preferably wherein the hyperinflammation and/or the immune paralysis is caused by an infectious disease such as COVID-19, by sepsis, myocardial infarction or stroke.
Statement 62. In vivo, in vitro or ex vivo use of IL-4 in stimulating or promoting trained immunity in a cell, organ, tissue or an organism.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The herein disclosed aspects and embodiments of the invention are further supported by the following non-limiting examples.

### EXAMPLES

### Example 1. Preparation of Fusion Proteins of an Apolipoprotein Fused to an Immunomodulatory Biomolecule and Incorporation Thereof into Lipid Nanoparticles

### Materials & Methods

Protein Expression, Purification, and Characterization of ApoA1-S147C (e.g. SEQ ID NO. 9) and ApoA1-S279C (e.g. SEQ ID NO. 11) Mutants, apoA1-IL4 Fusion Proteins (e.g. SEQ ID NO. 35), apoA1-IL2 (e.g. SEQ ID NO. 23) or apoA1-IL2v4 (e.g. SEQ ID NO. 31) Fusion Protein, apoA1-IL1p Fusion Protein (e.g. SEQ ID NO. 21), and apoA1-IL38 Fusion Protein (e.g. SEQ ID NO. 80) (FIGS. 1-9)

Bacterial cells containing the pET-vector coding for the desired protein were grown in 40 mL of 2YT medium supplemented with extra NaCl (10 g/L) and 100 μg/mL ampicillin was inoculated with transformed bacteria and grown overnight. The following day, 2YT medium containing 10 g/L NaCl and 100 μg/mL ampicillin was inoculated with the overnight culture and incubated at 37° C. at 150 rpm until an OD600 of 0.6-0.8 was reached. Isopropyl R-d-thiogalacopyranoside (IPTG) was added to a final concentration of 0.1 mM. The culture was further incubated at 20° C. at 150 rpm overnight. Bacteria were pelleted using centrifugation and lysed using Bugbuster® protein extraction reagent (Novagen) per gram cell pellet supplied with Benzonase® Nuclease (Merck), following the manufacturer's protocols. Lysates were centrifuged and resulting supernatant containing protein of interest was then processed on a IMAC column with Ni-NTA HisBind® Resin (Merck Millipore).

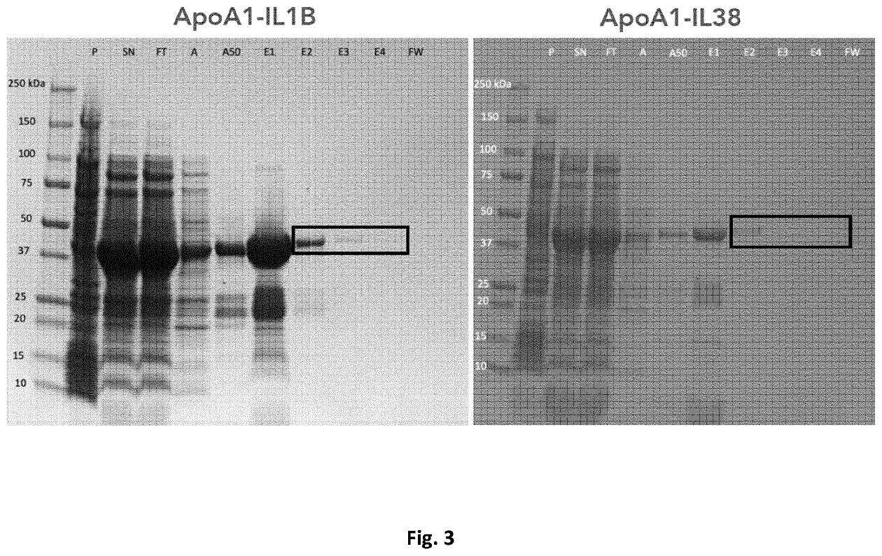

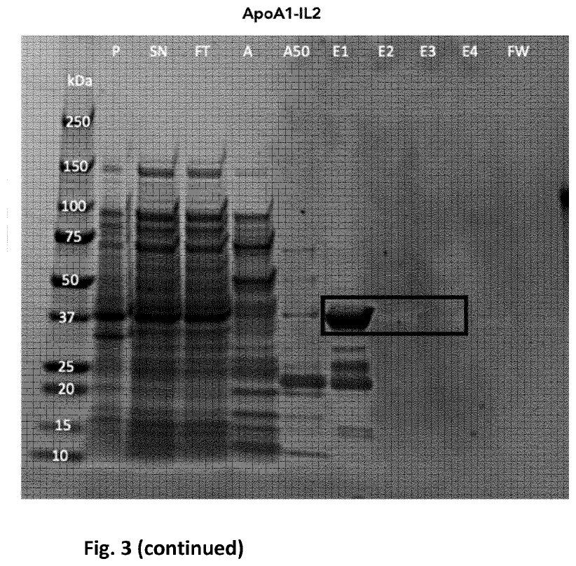

Obtained fractions were analyzed using SDS-PAGE (Samples were combined with sample buffer (1:1) and run on a Mini-PROTEAN® TGX™ Precast Gel (Bio-Rad). Resulting gels were stained using Coomassie G-250 Stain and de-stained using dH₂O FIGS. 3, 5).

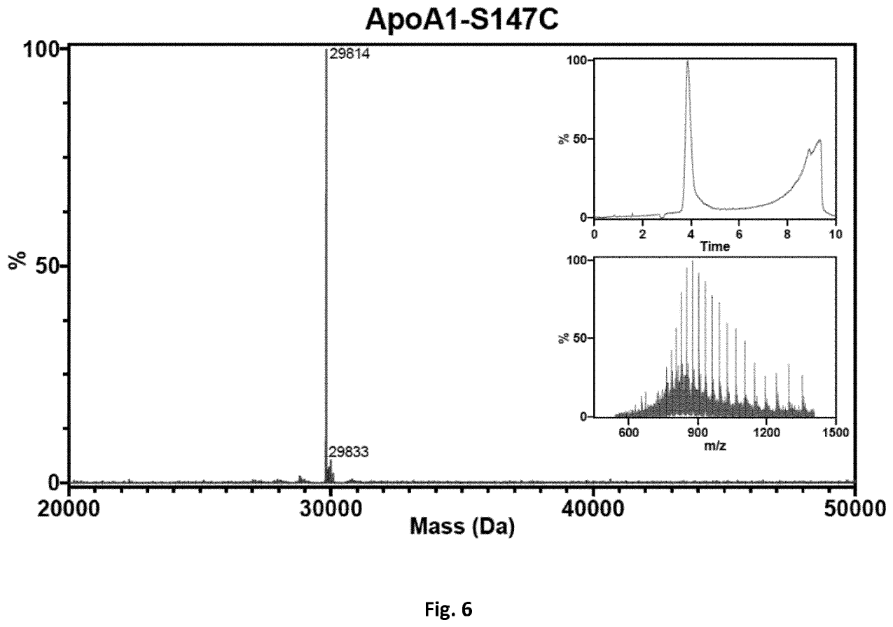

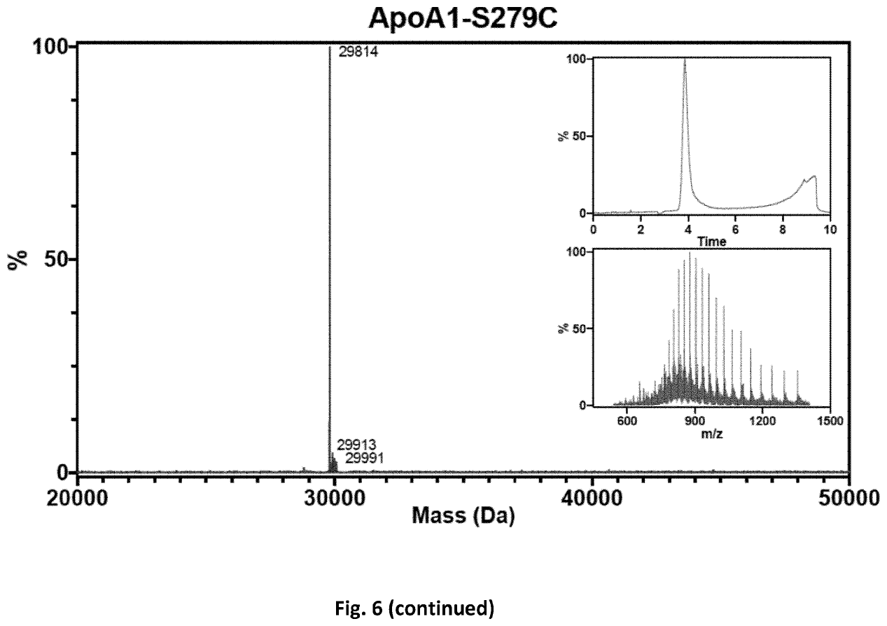

Obtained purified proteins of interest were further characterized using Q-ToF. Samples were diluted in 0.1% formic acid in dH2O to a concentration of 0.01 to 0.1 mg/mL. After filtration using a PD Spintrap™ G-25 column (0.5 mL, Cytiva), the samples were measured using a Waters ACQUITY UPLC I-Class system with a Xevo G2 Quadrupole Time of Flight mass spectrometer. The proteins were separated by a C8A reverse-phase column. A gradient of 15% to 75% acetonitrile in 0.1% formic acid in dH2O was used. Resulting data was analyzed using MassLynx (Waters) with the MaxEnt algorithm (FIG. 6).

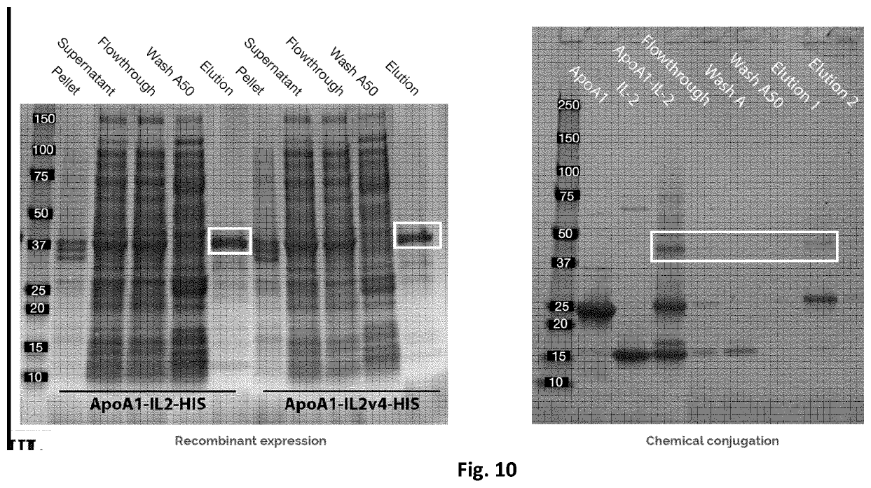

Protein Expression, Purification, and Characterization of ApoA1-S147C (e.g. SEQ ID NO. 9) and ApoA1-S279C (e.g. SEQ ID NO. 11) Mutants, apoA1-IL4 Fusion Proteins (e.g. SEQ ID NO. 35), apoA1-IL2 (e.g. SEQ ID NO. 58) or apoA1-IL2v4 (e.g. SEQ ID NO. 60) Fusion Protein, apoA1-IL1p Fusion Protein (e.g. SEQ ID NO. 21 or SEQ ID NO. 82), and apoA1-IL38 Fusion Protein (e.g. SEQ ID NO. 80 or SEQ ID NO. 84) (FIGS. 10-16)

Bacterial expression and protein purification): performed as described in the section “bacterial expression and protein purification” of Example 2.

Bacterial lysis and protein purification: performed as described in the section “bacterial lysis and protein purification” as described in Example 2.

### Discoidal Lipid Nanoparticle (LNP) Formulation

To formulate HDL-based LNPs, phospholipids, apoA1 fusion proteins and cholesterol are used. For discoidal LNPs, the fusion protein (in PBS) is mixed with a mixture of cholesterol and 1,2-dimyristoyl-sn-glycero-3-phosphocholine (DMPC) (in 95% acetonitrile/5% methanol) in a 1:10:100 molar ratio, using a staggered herringbone microfluidic mixer.

Dynamic Light Scattering measurements were performed on a Zetasizer Nano ZSP with the nanobiologics to assess size and heterogeneity. 100 μL of sample volume was pipetted into a transparent cuvette (Sarstedt) which was inserted into a Malvern Zetasizer Nano ZSP. The sample was measured in triplicate with 10 cycles per run at a temperature of 25° C.

Azide Introduction in Immunomodulatory Biomolecule and Conjugation to apoA1 or a Cysteine Mutant Thereof

ApoA1-IL4 fusion (for data represented in FIGS. 1-9): IL-4 was buffer exchanged to DEA buffer (50 mM diethanolamine, pH 7.5) and concentrated to 0.75 mM. Imidazole-1-sulfonyl azide hydrochloride (Fluorochem) was dissolved in dH2O to obtain a 20 mg/mL stock solution. The pH was adjusted to 7. This was then added to the IL-4 in a 227.5:1 molar ratio (17.5 molar equivalents per amine in IL-4). The reaction mixture was incubated at 4° C. overnight. The DBCO-PEG12-maleimide linker (Sigma-Aldrich) was dissolved in DMSO. The dissolved linker was then added to apoA1 S279C (0.5 mM) to a final concentration of 5 mM. This reaction mixture was also incubated at 4° C. overnight. The resulting functionalized IL-4-Az and apoA1-DBCO were then buffer exchanged to PBS (pH 7.9) to remove excess azide transfer reagent and DBCO-maleimide linker. The solutions were added together in a 1:2 apoA1: IL-4 molar ratio, and incubated at 4° C. overnight. The following day the mixture was purified on a IMAC column with Ni-NTA HisBind® Resin (Merck Millipore). Fractions were collected and analyzed using SDS-PAGE. Analysis was performed using Q-ToF (same method as described above) and a HEK293 IL-4 reporter assay.

ApoA 1-cytokine fusion (for data represented in FIGS. 10-16): Alternatively, stock solutions of imidazole-1-sulfonyl azide (2 mg/mL) in MQ were prepared. The pH of this stock was set to 7.5. To 100 μg cytokine in PBS (pH=7.5), 17.5 molar equivalents of imidazole-1-sulfonyl azide per primary amine were added. The reaction was incubated overnight at 4° C. After overnight incubation the excess imidazole-1-sulfonyl azide was removed by using a PD minitrap G-25 desalting column (Cytiva) and the resulting product was analyzed by Q-ToF LC-MS (WatersMassLynx v4.1), using MagTran V1.03 for MS. A 5× molar excess of a maleimide-PEG4-DBCO linker was added to ApoA1 or a cysteine mutant thereof and reacted overnight to form the apoA1-PEG4-DBCO complex. The excess linker was removed using an Amicon Ultra-0.5 Centrifugal Filter Unit (Merck) with MWCO 10 kDa. ApoA1-PEG4-DBCO was then combined with the azide-containing cytokine and incubated for 4 hours at RT or overnight at 4° C. Resulting product was analyzed using SDS-page.

### HEK293 IL-4 Reporter Assay

HEK-Blue™ IL-4 cells (Invivogen) were seeded in T25 culture flasks and grown at 37° C. until a confluency of 80% was reached. Cell viability was checked using a microscope. The cells were washed with sterile PBS and harvested using trypsinization for 5 minutes at 37° C. The cells were then seeded into a 96 wells plate so that each well contained 50.000 cells (180 μL DMEM, 10% FBS, 1% Pen-Strep). For each condition a 2-fold dilution series was prepared. The dilution series were added to the plate containing cells in triplo. The cells were then incubated for 24 hours at 37° C. A QUANTI-Blue™ (Invivogen) solution was prepared according to manufacturer's instructions. In another 96 wells plate, 20 μL of conditioned cell medium was added together with 180 μL of QUANTI-Blue™ solution. This was then incubated for 3 hours at 37° C. The absorbance was analyzed using a Spark® multimode microplate reader (Tekan) at 635 nm.

### Effect of IL2 Constructs on T-Cell Proliferation

Human CD3 positive T cells were stained with CFSE (Thermofisher) according to manufacturer's protocol. After incubation 100.000 T cells were seeded in 96 well round bottom plates and stimulated with IL2 or IL2 constructs for 6 days. T cells were harvested, washed, and stained for CD3, CD4 and CD8 and measured on Cytoflex (Beckman Coulter Inc.). Flow cytometry data was analyzed using FlowJo software (BD). Commercial 1L2 was purchased from Sinobiological. Recombinant IL2 was prepared as described below.

### Bacterial Expression of Recombinant IL-2

The SUMO-IL2 construct was transformed into Shuffle T7 competent E. Coli. 40 mL of 2YT medium containing 50 μg/mL kanamycin, was inoculated with bacteria and grown overnight at 250 rpm and 37° C. to form a small culture. The following day, 2YT medium containing 50 μg/mL kanamycin was inoculated with the small culture to form a large culture. The culture was incubated at 150 rpm and 37° C. until an OD600 of 0.6-0.8 was reached. Then to induce protein expression, IPTG was added to a final concentration of 0.1 mM. The culture was further incubated at 20° C. at 150 rpm overnight. Bacterial pellets were then obtained by centrifugation at 10.000×g for 10 minutes at 4° C. The resulting supernatant was discarded. The obtained pellet obtained was resuspended in 10 mL lysis buffer (20 mM TRIS, 500 mM NaCl, pH 7.9) per gram cell pellet. 25 U Benzonase® Nuclease (Merck) was then added. One cOmplete™ Protease Inhibitor Cocktail Tablet was added per 50 mL of extraction buffer. The resulting solution was stirred at 4° C. for 30 minutes until no clumps remained. The solution was then homogenized three times using the Avestin Emulsiflex C3 at 15.000-20.000 psi while being kept on ice. The cell lysate was then centrifuged at 20.000×g for 30 minutes at 4° C. The resulting supernatant was applied to an IMAC column, at 4° C., which was previously charged with an 0.1 M NiSO4 solution. All flowthrough fractions were collected. The column was washed with 8 column volumes of buffer A (20 mM Tris, 500 mM NaCl, 10 mM imidazole, pH 7.9), then 8 column volumes of buffer A50 (20 mM Tris, 500 mM NaCl, 50 mM imidazole, pH 7.9). To elute SUMO-IL2, 8 column volumes of buffer A500 (20 mM Tris, 500 mM NaCl, 500 mM imidazole, pH 7.9) was applied to the column. Obtained fractions were analyzed using SDS-PAGE. The eluted fractions containing SUMO-IL2 were pooled. SUMO hydrolase was added to the pooled fractions of SUMO-IL2 in a ratio of 1 mg hydrolase/500 mg protein. The solution was then dialyzed to storage buffer (20 mM TRIS, 500 mM NaCl, pH 7.9) using a Snakeskin™ 10 kDa cutoff dialysis bag (Thermo Scientific) while gently stirring at 4° C. overnight. Then the resulting protein solution was centrifuged at 4000×g for 20 minutes and the supernatant was filtered using a 0.2 μM syringe filter to remove aggregated protein. The IMAC column protocol was repeated and the fractions were again analyzed using SDS-PAGE. If the fraction containing IL2 was contaminated with other protein, the sample was purified using Size Exclusion Chromatography (SEC). Otherwise the fraction containing IL2 was buffer exchanged to PBS (pH 7.9). SEC was performed a NGC 10 Medium-Pressure Chromatography System (Bio-Rad) with a GE Hiload 16/60 Superdex 75 μg column. The column was first equilibrated using filtered PBS (pH 7.9) after which the sample was applied. The collected fractions were analyzed using SDS-PAGE. Fractions containing IL2 were pooled and the final concentration was determined using a Nanodrop™ 1000 spectrophotometer. The protein was then snap-frozen in liquid nitrogen, and stored at −80° C. Analysis was performed using Q-tof, and SPR.

Cryo-transmission electron microscopy (cryo-TEM): performed as described in Example 2.

Determining aNP size and dispersity by DLS: performed as described in Example 2.

SDS page: performed as described in Example 2

### Results

Our fusion proteins can be made by recombinant expression, or by chemical conjugation. Thus far, we have managed to express four different fusion proteins recombinantly. These are apoA1-IL4, apoA1-IL1B, apoA1-IL38 and apoA1-IL2. All expressions were successful and resulted in pure protein, as can be observed in FIG. 3. The black rectangle indicates the elution fractions containing our fusion protein of interest.

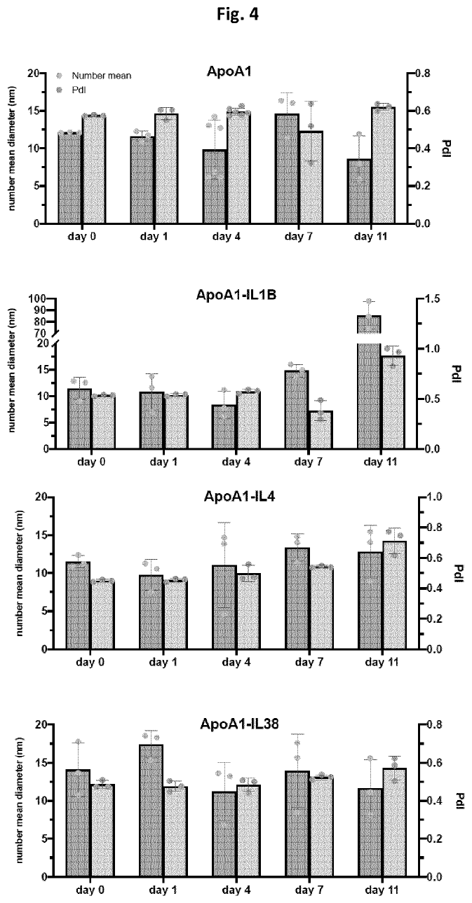

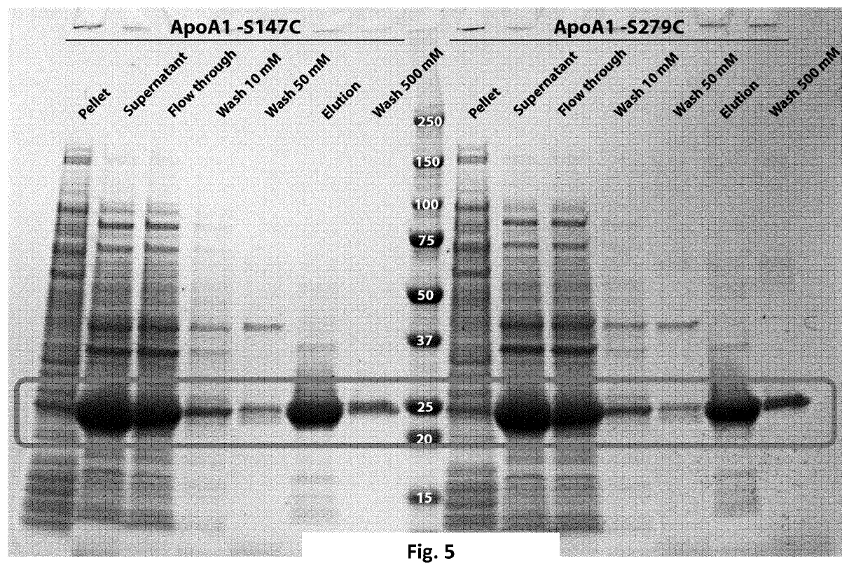

These recombinantly expressed proteins were then used to formulate nanobiologic discs (FIG. 4). It can be appreciated that apoA1-IL4 and apoA1-IL38 remain stable over 11 days and show similar diameters and PdI as apoA1. ApoA1-IL1b shows this as well, although after 11 days these nanobiologics appear to aggregate.

In order to chemically conjugate apoA1 to cytokines, nanobodies, or other biomolecules, a reactive handle is needed. Therefore, apoA1 mutants were created that contain a cysteine in the place of a serine at position 147 (referred to herein as the “S147C” or “S157C” mutant) or 279 (referred to herein as the “S279C” or “S239C” mutant). For example, an apoA1 mutant may be defined by a peptide sequence as set forth in SEQ ID NO. 9 or 11. Expression and purification of these proteins was successful, as can be seen in FIG. 5, and resulted in yields and purities similar to wild type apoA1. The obtained proteins were further characterized using quadrupole time-of-flight (Q-ToF), which showed that the protein was pure, and the correct mass was found (FIG. 6).

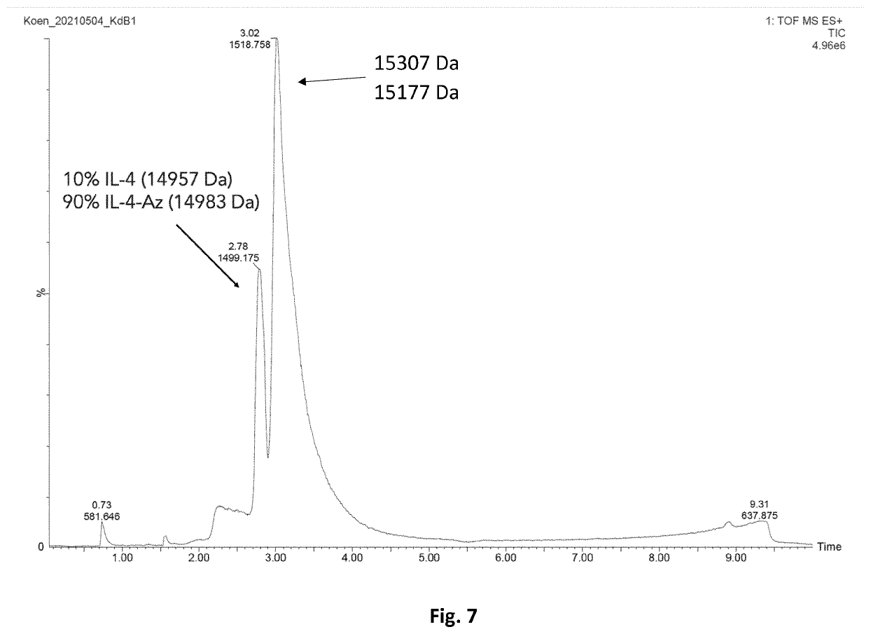

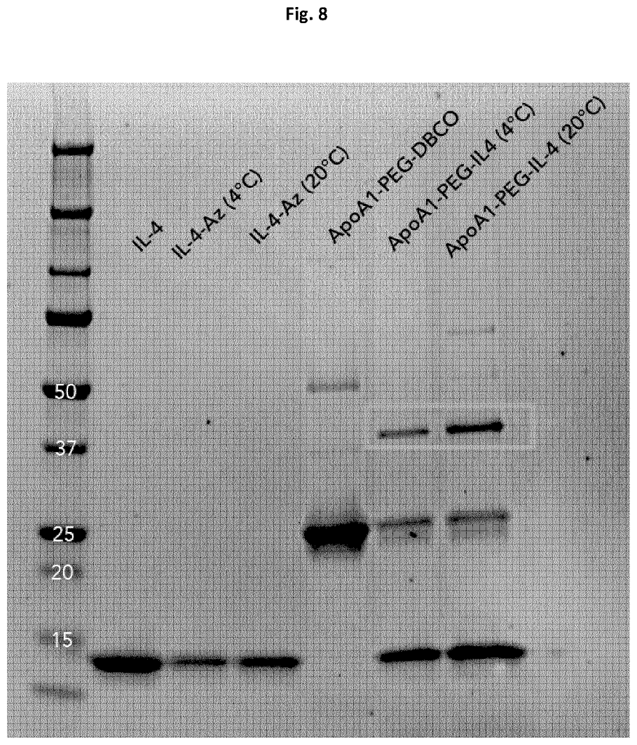

Conjugating cytokines to this apoA1 mutant can be done using a Maleimide-PEG-DBCO linker, where the maleimide couples to the apoA1 and the DBCO couples to an azide that we introduce in the cytokines. Introduction of a single azide at the N-terminus of IL-4 was optimized until ±90% of the product consisted of IL-4-Az (IL-4 with single azide incorporated). This was analyzed using Q-ToF (FIG. 7) and SDS-PAGE (FIG. 8). From the SDS-page it can be seen that upon conjugation of ApoA1S279C and IL4, a band appears around 40 kDa, corresponding to the expected molecular mass of the fusion protein. This confirms that we have chemically conjugated IL4 to apoA1 using a PEG-linker (FIG. 8). While this conjugation reaction is still being optimized, we have already got a yield of ±20%. The bioactivity of the recombinantly produced fusion proteins as well as the chemical apoA1-IL4 conjugate was then analyzed using a HEK293 IL-4 reporter assay. This cell line has a fully active STAT6 pathway and carries a STAT6-inducible secreted alkaline phosphatase (SEAP) reporter gene. Here, SEAP production is linked to binding of IL4 to its receptor. The HEK-Blue™ IL-4/IL-13 cells produce SEAP in response to IL-4 and IL-13. The IL-4 binding can be quantified by checking the enzymatic activity of SEAP using QUANTI-Blue colorimetric assay.

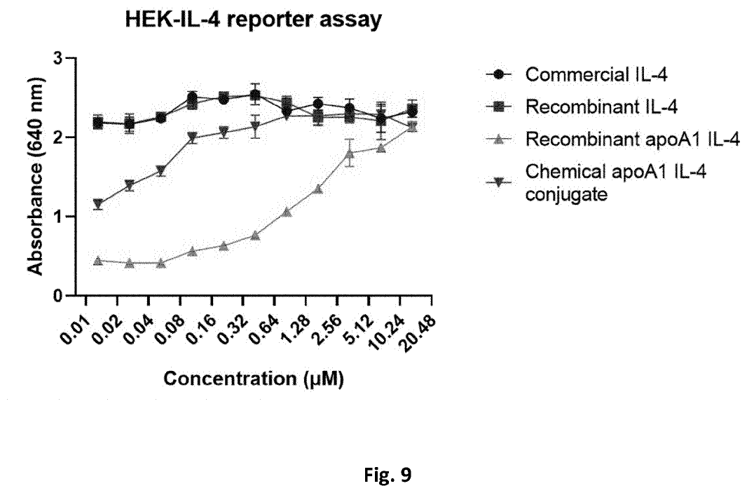

As can be seen from FIG. 9, both fusion proteins exert a dose-dependent effect and thus we can conclude that the IL4 in both fusion proteins is still functional. The chemically conjugated apoA1-IL4 preforms slightly less than the commercial and recombinant IL-4, which can be attributed to the fusion of apoA1 to IL-4. This likely hinders the binding of IL-4 to the receptor slightly. However, the chemically conjugated apoA1-IL4 performs better than the recombinant apoA1-IL4 protein, indicating that the affinity of the chemically conjugate apoA1-IL4 is likely higher than that of the recombinantly expressed apoA1-IL4.

### ApoA 1-IL2 Fusion Proteins

Besides the production of the apoA1-IL4 chemical and recombinant fusion proteins, we have also produced apoA1-IL2 fusion proteins. Here we have recombinantly fused wild-type IL2 or an IL2 mutant to apoA1. SDS-page analysis of the expressed and IMAC purified proteins indicated that the correct proteins are present in our elution fractions (FIG. 10, left panel). Additionally, we have also chemically conjugated wild-type IL2 to apoA1. This was also verified by SDS-page (FIG. 10, right panel).

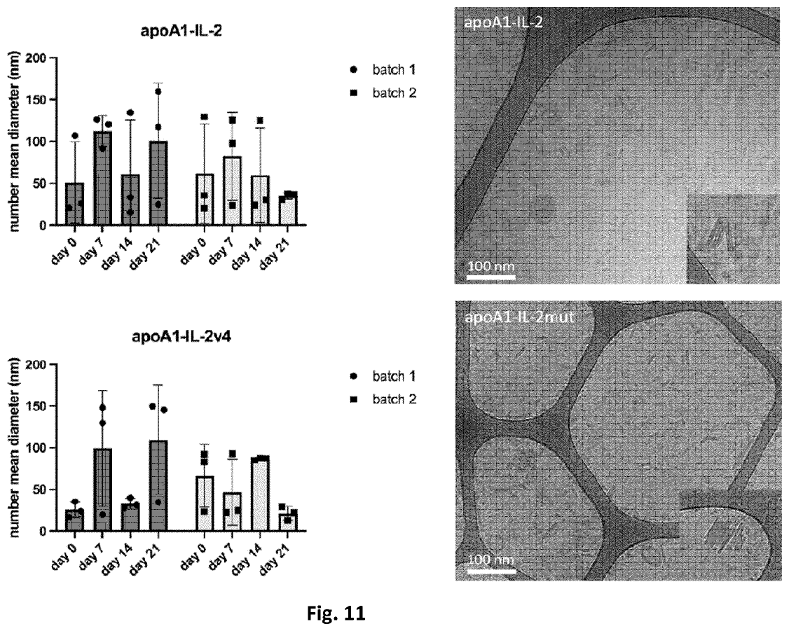

We have next integrated the apoA1-IL2 fusion proteins in discoidal lipid nanoparticles to yield IL2-aNPs and IL2v4-aNPs (FIG. 11). The successful formulation of discoidal nanoparticles was confirmed by cryogenic transmission electron microscopy (cryo-TEM) (FIG. 11, right panel). We additionally analyzed nanoparticle size and stability in PBS for 21 days using dynamic light scattering (DLS) (FIG. 11, left panel).

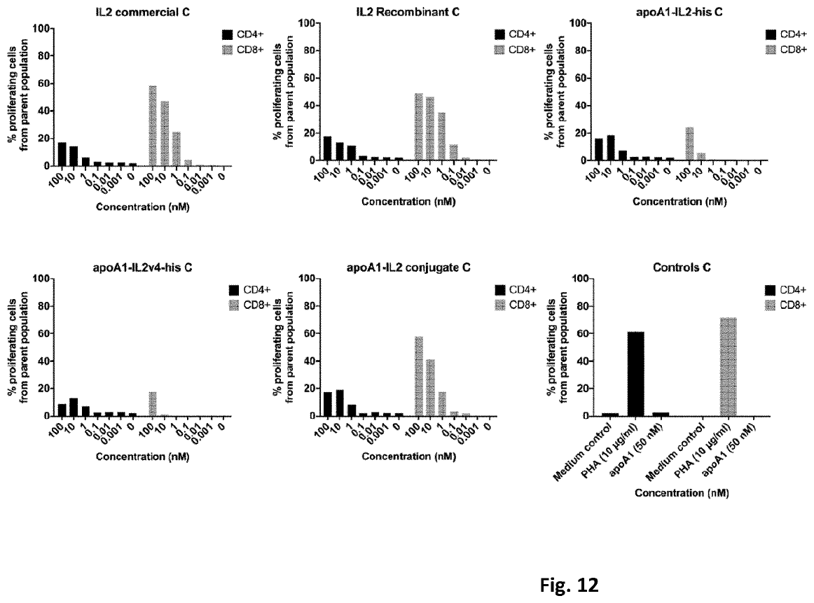

We assessed the ability of the apoA1-IL2 fusion proteins to stimulate T-cell proliferation. It can be seen that our fusion proteins are able to induce T-cell proliferation. Especially our apoA1-IL2 chemical conjugate (FIG. 12, middle bottom) can induce T-cell proliferation to a similar extend as commercially available IL2 (FIG. 12, top left).

### ApoA1-IL1β Fusion Proteins

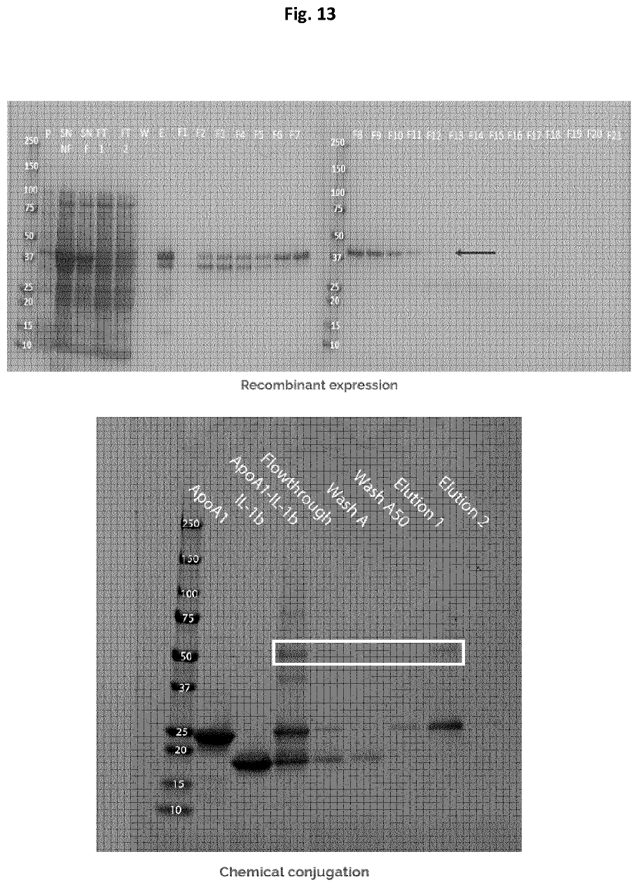

We further expanded our library by creating chemically conjugated and recombinantly expressed apoA1-IL1β fusion proteins. Here we have recombinantly fused IL1β to apoA1. SDS-page analysis of the expressed and IMAC purified protein showed that the correct protein is present in our elution fractions as indicated by the band at ˜40 kDa (FIG. 13, upper panel). Additionally, we have also chemically conjugated IL1β to apoA1. This was also verified by SDS-page (FIG. 13, lower panel).

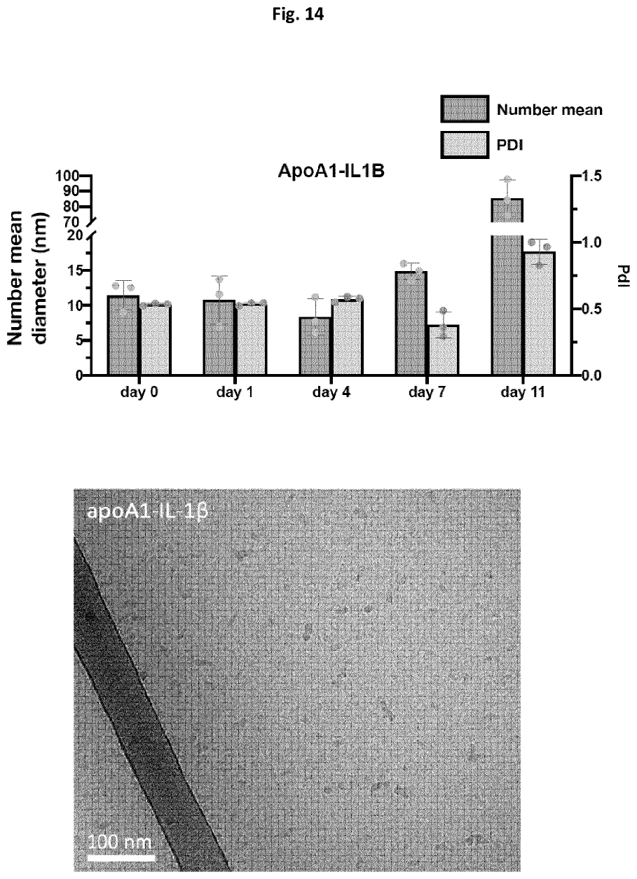

Next, we have integrated the apoA1-IL1β fusion protein in discoidal lipid nanoparticles to yield IL1β-aNPs. The successful formulation of discoidal nanoparticles was confirmed by cryogenic transmission electron microscopy (cryo-TEM) (FIG. 13, lower panel). We additionally analyzed nanoparticle size and stability in PBS for 21 days using dynamic light scattering (DLS) (FIG. 14, upper panel)

### ApoA 1-IL38 Fusion Proteins

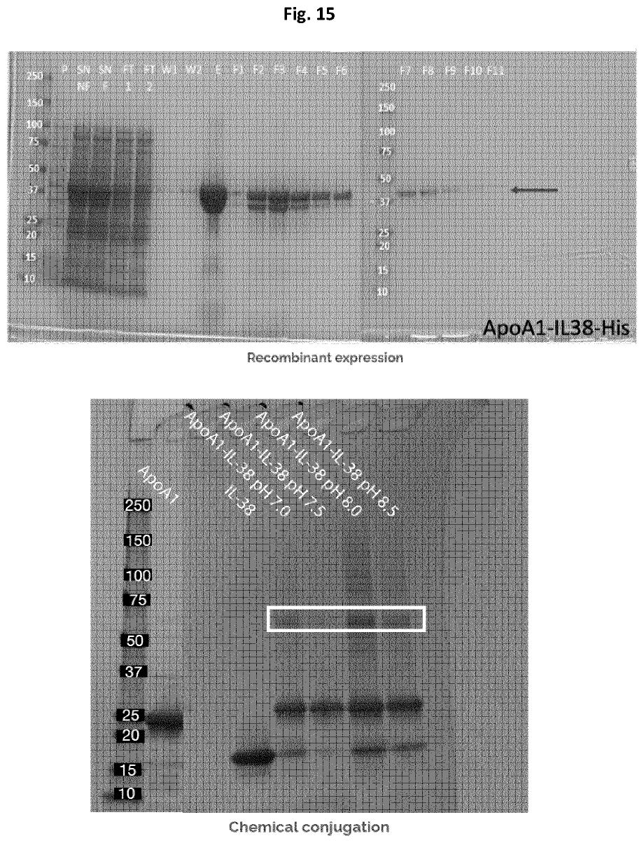

Additionally, we also chemically conjugated and recombinantly expressed apoA1-IL38 fusion proteins. Here we have recombinantly fused IL38 to apoA1. SDS-page analysis of the expressed and IMAC purified protein showed that the correct protein is present in our elution fractions as indicated by the band at ˜40 kDa (FIG. 15, upper panel). Additionally, we have also chemically conjugated IL38 to apoA1. This was also verified by SDS-page (FIG. 15, lower panel).

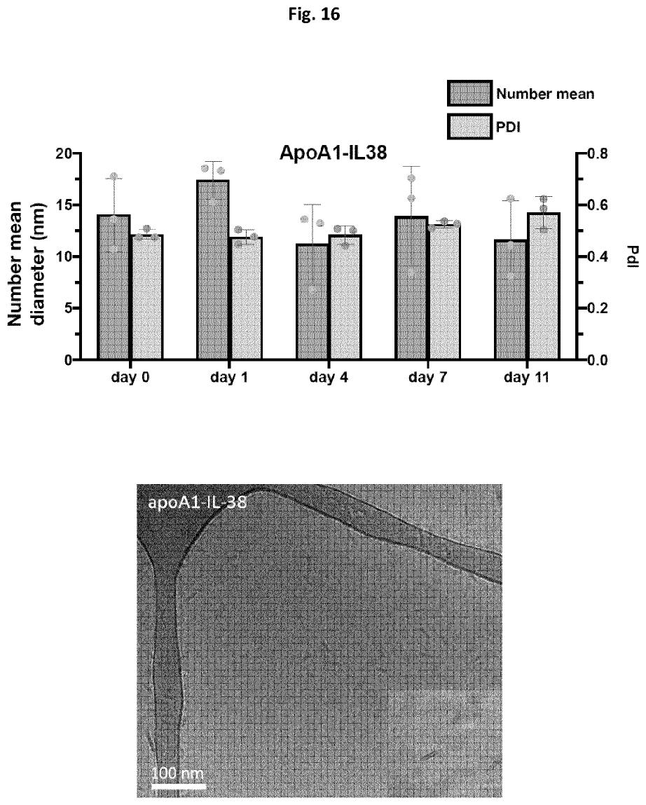

We have next integrated the apoA1-IL38 fusion protein in discoidal lipid nanoparticles to yield IL38-aNPs. The successful formulation of discoidal nanoparticles was confirmed by cryogenic transmission electron microscopy (cryo-TEM) (FIG. 16, lower panel)). We additionally analyzed nanoparticle size and stability in PBS for 21 days using dynamic light scattering (DLS) (FIG. 16, upper panel)

### Example 2. ApoA1-IL-4 Fusion Protein and Incorporation Thereof into Lipid Nanoparticles

### Materials and Methods

PBMC and monocyte isolation: Buffy coats (Sanquin) or EDTA whole blood from healthy volunteers was acquired after obtaining written informed consent. The material was diluted at least 1:1 with calcium/magnesium-free PBS (Lonza) and layered on top of Ficoll-Paque (GE Healthcare). Density-gradient centrifugation for 30 minutes at 615×g was used to separate the peripheral blood mononuclear cell (PBMC) interphase. Following 3-5 washes with cold PBS, PBMC yield and composition were assessed using a Sysmex hemoanalyzer (XN-450; Sysmex).

Negatively selected monocytes were obtained using MACS, according to the manufacturer's instructions (MACS Pan monocyte isolation kit, human; Miltenyi Biotec). Monocyte yield and purity was assessed on a Sysmex hemoanalyzer.

For some experiments (indicated in the text), monocytes were alternatively enriched from PBMCs by hyperosmotic density-gradient centrifugation over Percoll (Sigma-Aldrich). 150-200×106 PBMCs were layered on top of a hyperosmotic Percoll solution (48.5% v/v Percoll, 0.16 M NaCl, in sterile water) and centrifuged for 15 minutes at 580×g, RT. The interphase was collected, washed once with cold PBS, and resuspended in RPMI.

Primary human monocyte culture: All primary human monocytes/macrophages were cultured in RPMI-1640 with Dutch modifications (Invitrogen) which was further supplemented with GlutaMAX (2 mM; GIBCO), sodium pyruvate (1 mM; GIBCO), and gentamicin (50 μg/ml; Centrafarm). This medium is further referred to as RPMI+++. Additionally, 10% (v/v) human pooled serum was added to the medium during cell culture (also referred to as “cell culture medium”).

In vitro model of trained immunity in primary human monocytes: To induce trained immunity in primary human monocytes, a previously optimized and published method was used (Dominguez-Andres, J. et al. In vitro induction of trained immunity in adherent human monocytes. STAR Protoc 2, 100365, doi:10.1016/j.xpro.2021.100365 (2021); van Lier, D., Geven, C., Leijte, G. P. & Pickkers, P. Experimental human endotoxemia as a model of systemic inflammation. Biochimie 159, 99-106, doi:10.1016/j.biochi.2018.06.014 (2019)). Briefly: monocytes were adhered to a flat-bottom cell culture plate for 1 hour and washed with warm PBS to remove any non-adherent cells and cell debris. Then, they were stimulated (“trained”) for 24 hours with one of the stimuli detailed in Table 1, or medium only (“untrained” control).

TABLE 1
Stimuli for primary human monocytes and in vivo experiments.

| | Concentration in | | | |
|---|---|---|---|---|
| Stimulus | experiment | | Source | # |
| Recombinant human IL4 | 25 | ng/ml | R&D Systems | 6507-IL |
| (CHO-expressed) | | | | |
| C. albicans-derived β-glucan | 1 | μg/ml | Kindly provided by | — |
| | | | David Williams (TN, | |
| | | | USA) | |
| GM-CSF (premium grade) | 1000 | IU/ml | Miltenyi biotec | 130-093- |
| | | | | 864 |
| E. coli LPS serotype 055:B5; | 10 | ng/ml | Kindly prepared by | — |
| further purified as described | | | Heidi Lemmers, | |
| previously (Hirschfeld, M., Ma, | | | Radboudumc | |
| Y., Weis, J. H., Vogel, S. N. & | | | | |
| Weis, J. J. Cutting edge: | | | | |
| repurification of | | | | |
| lipopolysaccharide eliminates | | | | |
| signaling through both human | | | | |
| and murine toll-like receptor 2. J | | | | |
| Immunol 165, 618-622, | | | | |
| doi: 10.4049/jimmunol. 165.2.618 | | | | |
| (2000)) to specifically activate | | | | |
| TLR-4 | | | | |
| E. coli LPS serotype O55:B5 (for | 0.1 | mg/kg | Sigma | L2880- |
| in vivo experiments) | | | | 25MG |
| ApoA1-IL4 fusion protein | Molar equivalent of | | Prepared by D.P.S. | — |
| | 200 ng/ml IL4 | | | |
| IL4-aNP (discoidal and | Molar equivalent of | | Prepared by D.P.S. | — |
| spherical) | 200 ng/ml IL4 | | | |
| ApoA1-IL4ₘ fusion protein | | | Prepared by D.P.S. | |
| | | | and A.d.D. | |
| IL4ₘ-aNP (discoidal) | 200 | μg/mouse | Prepared by D.P.S. | |
| | | | and A.d.D. | |

For pharmacological inhibition experiments, the monocytes were pre-incubated for 1 hour with one of the inhibitors described in Table 2 before addition of the training stimulus.

TABLE 2
Inhibitors for primary human monocytes.

| | Concentration | | |
|---|---|---|---|
| Inhibitor (target) | in experiment | Source | # |
| Wortmannin (PI3K) | 100 nM | Invivogen | Tlrl-wtm |
| Torin-1 (mTOR) | 5 μM | Invivogen | Inh-tor1 |
| Cyproheptadine (SET7) | 100 μM | Selleckchem | S2044 |
| AS1517499 (STAT6) | 300 nM | Sigma | SML1906 |

Following the initial 24-hour stimulation, the cells were washed with warm PBS and warm cell culture medium was added. The monocytes were then allowed to rest and differentiate into macrophages for 5 days. On day 6, the induction of trained immunity was assessed. To this end, the cells were typically re-stimulated with LPS for an additional 24 hours to elicit cytokine production. The supernatants were collected and stored at −20° C. until further analysis, e.g. with ELISA for IL6 and TNF.

For most other trained immunity readout methods, the cells were harvested as follows: first, the cells were incubated in Versene cell dissociation reagent (Life technologies) for 30 minutes in a cell culture incubator. A cell scraper was then used to remove the cells from the culture plates. To maximize yield, the culture plates were scraped a second time after adding ice-cold PBS. The macrophages were centrifuged for 10 minutes at 300×g, 4° C. and counted before continuing to downstream applications.

In vitro inflammation inhibition: Monocytes were adhered to a flat-bottom cell culture plate for 1 hour and washed with warm PBS to remove any non-adherent cells and cell debris. Then, they were incubated with IL4 and LPS for 24 hours. Following the initial 24-hour stimulation, the supernatants were collected and stored at −20° C. until further analysis with ELISA for IL6 and TNF.

Primary monocyte-derived dendritic cell (moDC) generation: For experiments were moDCs were compared to macrophages (untrained control or IL4-trained), moDCs were differentiated as follows. First, negatively selected monocytes were obtained as described above. Following 1h adherence and a PBS wash, they were cultured in RPMI+++ with 10% HPS, further supplemented with IL4 (25 ng/ml) and GM-CSF (1000 IU/ml; premium grade, Miltenyi Biotec). The cells were differentiated until day 6, with one medium refresh on day 3. On day 6, the non-adherent cells were harvested in addition to the adherent cells (as described above). The moDCs and macrophages were then subjected to analysis by flow cytometry as described below.

In vivo experimental human endotoxemia model and ex vivo analyses: Eight healthy (as confirmed by medical history, physical examination, and routine laboratory testing) male volunteers provided written informed consent to participate in experimental endotoxemia experiments conducted at the research unit of the intensive care department of the Radboud university medical center. All study procedures were approved by the local ethics committee (CMO Arnhem-Nijmegen, registration numbers NL71293.091.19 and 2019-5730), and were conducted in accordance with the latest version of the declaration of Helsinki.

A continuous endotoxin infusion regimen was employed, as is described in detail elsewhere (van Lier, D., Geven, C., Leijte, G. P. & Pickkers, P. Experimental human endotoxemia as a model of systemic inflammation. Biochimie 159, 99-106, doi:10.1016/j.biochi.2018.06.014 (2019)). In short: subjects were admitted to the research unit and an antecubital vein and radial artery were cannulated to allow administration of fluids and endotoxin, and blood sampling and hemodynamic monitoring, respectively. A 3-lead ECG was recorded continuously throughout the experiment. After iso-osmolar pre-hydration (1.5 L NaCl 0.45%/glucose 2.5% administered intravenously in the hour before start of endotoxin infusion), volunteers were intravenously challenged with a loading dose of 1 ng/kg bodyweight endotoxin (E. coli lipopolysaccharide [LPS] type 0113, lot no. 94332B1; List Biological laboratories), directly followed by continuous infusion of 0.5 ng/kg/hour for 3 hours. Participants were monitored for 8 hours after the endotoxin loading dose after which they were discharged from the research unit.

For this project, blood samples were obtained at two timepoints: 1 hour before, and 4 hours after administration of the loading dose. Negatively selected monocytes were acquired as described above. The cells were adhered and stimulated for 24 hours with recombinant human IL4, discoidal IL4-aNPs, LPS (to assess initial immune tolerance), or medium only (as a control). Following a PBS wash, the cells were rested in culture medium for 48 hours and re-stimulated with LPS for an additional 24 hours. Supernatants were collected and stored at −20° C.

Cytokine and lactate measurements: TNF, IL6, and IL1Ra were measured in cell culture supernatants using duoset ELISA kits (R&D systems), according to the manufacturer's instructions. For lactate measurements, a fluorometric assay was used. 30 μl sample, medium control, or known standard was added to a black 96-well plate. Then, 30 μl reaction mix (PBS pH 7.4, horse radish peroxidase (0.2 U/ml), lactate oxidase (2 U/ml), Amplex red (100 μM; Fisher scientific)) was added and the reaction was incubated for 20 minutes in the dark at RT. Immediately thereafter, fluorescence was measured at 530/25 nm and 590/35 nm. Gen5 software (v3.03, BioTek) was used in conjunction with Microsoft Excel to calculate cytokine and lactate concentrations in the original samples.

Macrophage surface marker flow cytometry: Macrophages were harvested as described above and transferred to a v-bottom 96 well plate for staining. The cells were centrifuged at 1500 rpm, 5 minutes, 4° C. The supernatant was removed, and the cells were washed once with 200 μl PBA (PBS pH 7.4, 1% w/v BSA (Sigma)).

Fc-receptors were blocked by incubation in PBS supplemented with 10% human pooled serum for 15 minutes at 4° C. After washing once more, surface markers and viability were stained for in a volume of 50 μl for 30 minutes at 4° C., using the antibodies and viability dye described in table 3.

TABLE 3
Flow cytometry antibodies for experiments with primary human monocytes/macrophages
(and MLR experiments).

| Target | Clone | Fluorophore | Source | # | Assay (Figure) |
|---|---|---|---|---|---|
| CD80 | 2D10 | AF488 | Biolegend | 305214 | Surface markers |
| CD14 | M5E2 | PE | Biolegend | 301806 | Surface markers |
| CD206 | 15-2 | BV421 | Biolegend | 321126 | Surface markers |
| CD200R | OX108 | AF647 | Biorad | MCA2282T | Surface markers |
| Viability | Fixable | eFluor780 | Thermo Fisher | 65-0865 | Surface markers |
| | viability | | Scientific | | |
| | dye | | | | |
| CD45 | HI30 | BV510 | Biolegend | 304036 | Phagocytosis |
| Candida | — | FITC | Kindly provided | | Phagocytosis |
| albicans | | | by Dr. Martin | | |
| | | | Jaeger, | | |
| | | | Radboudumc | | |
| CD1c | L161 | BV421 | Biolegend | 331526 | moDC |
| | | | | | comparison |
| Viability | Fixable | eFluor506 | Thermo Fisher | 65-0866 | moDC |
| | viability | | Scientific | | comparison |
| | dye | | | | |
| IFNγ | B27 | BV421 | BD | 562988 | MLR (T cell) |
| IL4 | 7A3-3 | PE | Miltenyi | 130-091- | MLR (T cell) |
| | | | | 647 | |
| IL10 | JES3-9D7 | VioB515 | Miltenyi | 130-108- | MLR (T cell) |
| | | | | 100 | |
| IL17 | BL168 | AF647 | Biolegend | 512310 | MLR (T cell) |
| CD4 | RPA T4 | PerCP | Biolegend | 300528 | MLR (T cell) |
| CD8 | RPA T8 | FITC | BD | 555366 | MLR (T cell) |
| Granzyme | QA16A02 | PE | Biolegend | 377208 | MLR (T cell) |
| B | | | | | |
| Perforin | dG9 | AF647 | Biolegend | 308110 | MLR (T cell) |
| pSTAT6 | CHI2S4N | PE | eBioscience | 12-9013-42 | pSTAT6 |

Following two washes, the cells were resuspended in 150 μl PEA and measured on a Cytoflex flow cytometer (Eeckman Coulter) or ED FACSVerse system (ED Eiosciences). Compensation was performed using VersaComp compensation beads (Eeckman Coulter) for single antibody stains; a mixture of live and heat-killed cells was used for single stains of the viability dye (as per the manufacturer's recommendations). Data analysis was performed in Flowjo (v10.7.1, ED Eiosciences) Our gating strategy was as follows: first, a time gate was used if necessary. Then, single cell events were selected using subsequent FSC-A/SSC-A and FSC-A/FSC-H gates. Dead cells were removed from the analysis by selecting the viability dye-negative population. Geometric mean fluorescence intensities were calculated as a measure of surface marker expression.

T cell polarization readout: For MLR experiments, harvested macrophages were used for subsequent T cell polarization assays. Allogeneic naïve T cells were seeded with macrophages in a ratio of 10 T cells for every macrophage. The cells were cultured in flat-bottom 96 well plates for 7 days in standard cell culture medium. In this model, HLA mismatch causes non-specific activation of the T cell receptor. On the final day, the cells were stimulated with PMA (25 ng/mL)+ionomycin (0.5 μg/mL) for 4 hours in the presence of 100 ng/mL Brefeldin A, a ‘golgi-plug’. The cells were harvested and split over 2 flow-cytometry antibody panels (one for CD4 T cells and one for CD8; see also table 3). The cells were stained in a similar manner as described above, with an extra step for permeabilization of the T cells to allow for intracellular cytokine staining. This was performed using the Fix/Perm buffer set (eBioscience), according to the manufacturer's instructions. The gating strategy was similar to what is described above, with the addition of a selection for CD3-positive events. The percentage of cells positive for hallmark cytokines of T cell polarization were calculated to estimate T cell subset proportions.

Phospho-STAT6 measurement by flow cytometry: Monocytes were stimulated with RPMI, IL4, or different concentrations of IL4-aNPs (indicated in the figure) for 20 minutes at 37° C. The cells were transferred to a v-bottom 96 wells plate and kept on ice for the duration of the staining procedure. After staining for viability and CD14 (in the manner described above), the cells were fixed and permeabilized using the fix/perm buffer set (eBioscience) for 45 minutes at 4° C. in the dark. The cells were washed twice with perm buffer and incubated overnight in freezer-chilled absolute methanol at −20° C. overnight. Following two more washes in perm buffer the cells were stained for phospho-STAT6 using the antibody described in table 3, for 45 minutes at 4° C. in the dark. The cells were washed two more times in perm buffer and finally resuspended in PBA for acquisition on the Cytoflex cytometer. The gating strategy was largely similar to the one for macrophage surface marker with the addition of a selection for CD14-positive events.

Phagocytosis assay: Macrophages were harvested as described above and incubated at 37° C. for 1 hour with FITC-labeled Candida albicans (kindly provided by Dr. Martin Jaeger, Radboudumc) at an MOI of 1:5. The cells were washed 2 times with ice-cold PBA and kept on ice to halt the phagocytosis. The cells were stained for CD45 (table 3) during 30 minutes in the dark at 4° C. Following two washes, trypan blue was added to a final concentration of 0.01% to quench extracellular FITC-Candida. The cells were then acquired on a Cytoflex flow cytometer.

During data analysis, CD45+ events were first selected to remove Candida-only events. Single cells were then gated on as described above and the percentage of Candida-FITC positive macrophages in each sample was calculated.

Seahorse metabolic analyses: Macrophages were harvested as described above. The cells were resuspended in RPMI+++ and seeded into overnight-calibrated cartridges at 10⁵ cells per well. After adhering for 1 hour, the medium was changed to assay medium (Agilent; see below) and the cells were incubated for 1 hour at 37° C. in ambient C02 levels. Oxygen consumption rates (OCR) and extracellular acidification rates (ECAR) were measured as proxies for glycolytic and mitochondrial metabolism, using a Seahorse XF Glycolysis Stress Test kit or a Seahorse XF Cell Mito Stress Test kit (both Agilent; measurements performed according to manufacturer's instructions).

RNA isolation, sequencing, and analysis: Monocytes or macrophages were lysed in RLT buffer (Qiagen) and stored at −80° C. RNA extractions were performed using RNeasy mini columns (Qiagen) with on-column DNAse I treatment (RNase-free; Qiagen). Preliminary quality control and measurements of concentration were performed using a Nanodrop apparatus. Samples were sent to the Beijing Genome Institute (BGI Denmark) for RNA sequencing using the DNBseq platform.

To infer gene expression levels, RNA-seq reads were aligned to hg19 human transcriptome using Bowtie. Quantification of gene expression levels as RPKM was performed using MMSEQ. Reads/transcript were normalized using DEseq2 and pair-wise comparisons were performed. Differentially expressed genes were identified using DEseq2 with fold change >2 and p-value <0.05, with a mean RPKM >1. To identify genes that were upregulated or attenuated by IL4 training, RPMI-d6 and IL4-d6 macrophages were compared with RPMI-d6+LPS and IL4-d6+LPS samples, respectively. Gene lists were merged and ranked based on IL4-d6+LPS/RPMI-d6+LPS. Gene ontology and TF motif analysis was performed on gene promoters using the HOMER findMotifs tool.

Chromatin immunoprecipitation: Macrophages were harvested as described above and resuspended in RPMI+++. The cells were fixed for 10 minutes in 1% methanol-free formaldehyde. The reaction was then quenched for 3 minutes by adding 125 mM glycine. The fixed cells were washed three times with ice-cold PBS and lysed at approximately 15*10⁶ cells/ml in lysis buffer (20 mM HEPES pH 7.6, 1% SDS, 1× protease inhibitor cocktail (PIC; Roche)), sonicated (Bioruptor Pico, Diagenode), and centrifuged (10 minutes, 13000 rpm, RT).

Aliquots of chromatin were de-crosslinked in 0.5×TBE buffer (supplemented with 0.5 mg/ml proteinase K (Qiagen)) for 1 hour at 65° C. and run on a 1% agarose gel to confirm target fragment size of 200-800 bp.

The remaining chromatin was divided into ChIP and input samples. ChIP samples were diluted 10× in dilution buffer (16.7 mM Tris pH 8.0, 1.0% Triton, 1.2 mM EDTA, 167 mM NaCl, 1×PIC in MilliQ) and 1 μg of ChIP-grade antibody (Diagenode) was added. The samples were rotated overnight at 4° C.

Magnetic protein A/G beads (Dynabeads) were washed 2 times in dilution buffer supplemented with 0.15% SDS and 0.1% BSA. The washed beads were added to the ChIP samples and rotated at 4° C. for 1 hour. The bead-bound chromatin was subsequently washed (rotation for 5 minutes, 4° C.) as follows: 1× with low-salt washing buffer (20 mM Tris pH 8.0, 1.0% Triton, 0.1% SDS, 2 mM EDTA, 150 mM NaCl in MilliQ); 2× with high-salt washing buffer (same as low-salt washing buffer but with 500 mM NaCl); 2× with no-salt washing buffer (20 mM Tris pH 8.0, 1 mM EDTA, in MilliQ). Chromatin was eluted from the beads in elution buffer (0.1 M NaHCO₃, 1% SDS, in MilliQ) for 20 minutes, RT. Input samples were diluted 12× in elution buffer. After addition of NaCl (0.2 M) and proteinase K (0.1 mg/ml), all samples were decrosslinked for at least 4 hours on a shaking heatblock (65° C., 1000 rpm). Minelute PCR purification columns (Qiagen) were used to purify DNA fragments. DNA fragments were stored at 4° C. until downstream analysis by qPCR.

qPCR and analysis: qPCR analysis for ChIP samples and inputs was performed as follows. The SYBR green method was used to perform qPCR with the primers detailed in table 4. A comparative Ct method was used to compare ChIP against input samples and calculate relative abundance over a negative control region. GAPDH and the untranslated region of ZNF were respectively used as negative and positive controls for H3K9me3. TNF was interrogated using 6 primer pairs for AUC analysis as described previously (Bekkering, S. et al. Treatment with Statins Does Not Revert Trained Immunity in Patients with Familial Hypercholesterolemia. Cell Metabolism 30, 1-2, doi:10.1016/j.cmet.2019.05.014 (2019)).

TABLE 4
Primers for ChIP-qPCR analysis.

| Target | Forward (5′-3′) | Reverse (5′-3′) |
|---|---|---|
| ZNF UTR | AAGCACTTTGACAACCGTGA | GGAGGAATTTTGTGGAGCAA |
| | (SEQ ID NO. 62) | (SEQ ID NO. 63) |
| GAPDH | CCCCGGTTTCTATAAATGAGC | AAGAAGATGCGGCTGACTGT |
| | (SEQ ID NO. 64) | (SEQ ID NO. 65) |
| TNF (1) | AGAGGACCAGCTAAGAGGGA | AGCTTGTCAGGGGATGTGG |
| | (SEQ ID NO. 66) | (SEQ ID NO. 67) |
| TNF (2) | CAGGCAGGTTCTCTTCCTCT | GCTTTCAGTGCTCATGGTGT |
| | (SEQ ID NO. 68) | (SEQ ID NO. 69) |
| TNF (3) | GTGCTTGTTCCTCAGCCTCT | ATCACTCCAAAGTGCAGCAG |
| | (SEQ ID NO. 70) | (SEQ ID NO. 71) |
| TNF (4) | TGTCTGGCACACAGAAGACA | CCCTGAGGTGTCTGGTTTTC |
| | (SEQ ID NO. 72) | (SEQ ID NO. 73) |
| TNF (5) | AGCCAGCTGTTCCTCCTTTA | TTAGAGAGAGGTCCCTGGGG |
| | (SEQ ID NO. 74) | (SEQ ID NO. 75) |
| TNF (6) | TGATGGTAGGCAGAACTTGG | ACTAAGGCCTGTGCTGTTCC |
| | (SEQ ID NO. 76) | (SEQ ID NO. 77) |

Bacterial expression and protein purification: ClearColi BL21 (DE3) (Lucigen) were transformed with a pET20b(+)ApoA1-IL4 expression vector. Transformed bacteria were inoculated in 40 mL lysogeny broth (LB) (Sigma-Aldrich) supplemented with 100 μg/L ampicillin and grown overnight at 37° C. Subsequently, the overnight culture was inoculated in 2YT medium (16 g/L Peptone, 10 g/L yeast extract and 10 g/L NaCl) supplemented with 100 μg/L ampicillin and grown at 37° C. At the point that absorbance at 600 nm reached >1.5, 1.0 mM isopropyl β-d-thiogalacopyranoside (IPTG) was added to induce pET20b(+)ApoA1-IL4 expression, cells were incubated overnight at 20° C. Cells were harvested by centrifugation before preparation of lysates and purification.

Bacterial lysis and protein purification: ApoA1-IL4 fusion protein expressing ClearColi cells were harvested by centrifugation at 8000 rpm and 4° C. for 10 minutes. Harvested cells were resuspended in PBS and centrifuged at 4000 rpm and 4° C. for 15 minutes. Cells were lysed using 20 mL BugBuster® Protein Extraction Reagent (Merck) and 20 μL Benzonase® Nuclease (Merck) per liter culture on a shaker for 30 min at RT. Cell lysates were centrifuged at 18000 rpm and 4° C. for 30 minutes. Insoluble pellets were washed with 10 mL BugBuster per liter and centrifuged at 18000 rpm and 4° C. for 20 minutes. Pellet containing inclusion bodies was resuspended in extraction buffer (6 M guanidine hydrochloride, 50 mM potassium phosphate and 1 mM reduced glutathione) and incubated on a shaker for 15 min at RT. Suspension was centrifuged at 18000 rpm and 4° C. for 30 minutes to remove insoluble fraction. Filtered soluble fraction was loaded on a nickel column and washed with 15 column volumes IMAC wash buffer. ApoA1-IL4 was refolded on the nickel column using a linear gradient unfolding 60 mL (7 M urea, 1 mM reduced glutathione, 0.1 mM oxidized glutathione, 50 mM potassium phosphate and 100 mM NaCl pH 6.8) to refolding 60 mL (1 mM reduced glutathione, 0.1 mM oxidized glutathione, 50 mM potassium phosphate and 100 mM NaCl pH 6.8) 2.5 mL/minute. Refolded apoA1-IL4 was eluted from the column with 0.5 M imidazole, 20 mM Tris, 0.5 M NaCl pH 7.9. Eluate was collected, concentrated, and further purified and buffer-exchanged via size exclusion chromatography (HiLoad 16/600 Superdex 75 Increase; GE Healthcare) equilibrated with PBS storage buffer. Fractions were analyzed by SDS-PAGE, pooled, concentrated, and snap-frozen in liquid nitrogen before storing at −80° C. ApoA1-IL4 mass was confirmed by Q-ToF LC-MS (WatersMassLynx v4.1), using MagTran V1.03 for MS.

Mammalian expression and purification of apoA1-IL4m: HEK293T cells were co transfected with fuGENE (Promega) including transfer vector pHR-apoA1-IL4m, packaging pCMVR8.74 and envelop pMD2.G in Opti-MEM (GIBCO) at 37° C. for 24 hours. Cells were washed with DMEM+2% heat inactivated FBS and incubated for 48 hours. To obtain the lentivirus containing pHR-apoA1-IL4m, supernatant was centrifuged at 1000 rpm to remove cell debris filtered through 0.45 μm PES syringe filter and centrifuged at 50,000 g for 2 hours at 4° C. Pellet containing pHR-apoA1-IL4m lentivirus was resuspend in culture medium, snap frozen in liquid nitrogen and sorted at −80° C. HEK293F cells were transduced with pHR-apoA1-IL4m containing lentivirus in transfection medium (DMEM, 10% HI FBS, 1× Polybrene (Sigma-Aldrich) for 24 hours. Subsequently, cells were cultured in expression medium (50% EX-CELL® 293 Serum-Free Medium for HEK293 Cells (Merck) and 50% FreeStyle™ 293 Expression Medium (Thermo Fisher Scientific), supplemented with Glutamax, 1% Pen-Strep and 1 μg/mL doxycycline (Merck) on a shaker at 150 rpm for 3 days at 37° C. Culture supernatant containing apoA1-IL4m was centrifuged at 4000 rpm, 4° C. for 15 minutes and filtered through 0.22 μm PES syringe filter to remove cell debris. Filtered soluble fraction was loaded on a StrepTactin XT 4flow 5 mL column (Cytiva) and washed with 5 column volumes W-buffer (150 mM NaCl, 100 mM Tris, 1 mM EDTA pH 8) with flow rate 1-2 mL/minute. ApoA1-IL4m was eluted from the column with W-buffer supplemented with 50 mM biotin. Eluate was collected, concentrated, and snap-frozen in liquid nitrogen before storing at −80° C. Apoa1-IL4m mass was confirmed by Q-ToF LC-MS (WatersMassLynx v4.1), using MagTran V1.03 for MS.

SDS-PAGE and Western blot: To confirm fusion of apoA1 and IL4, 100 ng IL4 (BioLegend), apoA1 and apoA1-IL4 were loaded on a 4-20% polyacrylamide gel (Bio-Rad). After gel electrophoresis, samples were transferred to nitrocellulose membranes with blot buffer (10× TG buffer, 20% methanol). Subsequently, membranes were incubated with blocking buffer (5% milk, 0.1% tween in PBS (PBST)) overnight at 4° C. The blots were incubated with primary monoclonal antibodies monoclonal anti-IL4 (HIL41, 1:200; sc-12723, Santa Cruz Biotechnology) and anti-apoA1 (B10, 1:100; sc-376818, Santa Cruz Biotechnology) for 1 hour at 4° C. After incubation with primary antibodies, membranes were washed and incubated with rabbit anti-mouse IgG (H+L)-HRP conjugate (1:5000, 31457, Pierce). HRP-conjugated secondary antibodies were detected with TMB (Thermo Fisher Scientific) and visualized using the Image Quant gel imager (GE Healthcare).

Surface plasmon resonance: SPR measurements were performed using a Biacore X100 SPR system (GE Healthcare). Human IL4 receptor alpha-FC chimera (Biolegend) was immobilized on a protein G sensor chip (GE Healthcare). Log 2 dilution concentration series of apoA1-IL4 ranging from 200 nM to 6.25 nM and of human IL4 ranging from 20 nM to 0.65 nM. All samples were prepared in HPS-EP buffer (10 mM HEPES, 150 mM NaCl, 3 mM EDTA, 0.005% (v/v) P20 pH 7.4). Association was monitored for 180 seconds and dissociation for 180 seconds with a flow rate of 30 μL/minute. Sensor chip was regenerated with glycine 1.5 (10 mM glycine-HCl pH 1.5, GE Healthcare). Kinetics was determined by fitting the interaction SPR data for 1:1 binding.

Human embryonic kidney 293 IL4 reporter cell assay: HEK-Blue™ IL4/IL13 cells were purchased from InvivoGen. This cell line has a fully active STAT6 pathway and carries a STAT6-inducible SEAP reporter gene. The HEK-Blue™ IL4/IL13 cells produce SEAP in response to IL4 and IL13. The levels of secreted SEAP can be determined with QUANTI-Blue™ (Invivogen). 180 μL DMEM with 10% FBS and 1% Pen-Strep containing 5*104 cells was added per well in a 96-well-plate. Subsequently, 20 μL stimulus or vehicle was added and cells were incubated for 20-24 hours at 37° C. Subsequently, 180 μL QUANTI-Blue was added per well to a separate 96-well-plate (flat-bottom) and 20 μL of the cell supernatant was added. The plate was incubated for 1-3 hours at 37° C. and absorbance at 640 nm was measured on a Tecan Spark plate reader to determine SEAP levels.

Formulating nanoparticles: All phospholipids were purchased from Avanti Polar Lipids Inc.

Four different apoA1 based nanoparticles (aNPs) were formulated. For discoidal aNPs from stock solutions (10 mg/mL) in chloroform, DMPC (133.5 μL), cholesterol (Sigma-Aldrich) (7.5 μL) and for spherical aNPs, POPC (66.5 μL), PHPC (17.5 μL), cholesterol (4.5 μL), and tricaprylin (Sigma-Aldrich) (2.79 μL from 0.956 g/mL stock) were combined in a glass vial and dried under vacuum. The resulting film was redissolved in an acetonitrile/methanol mixture (95:5%, 800 μL total volume). For formulation based on apoA1, cholesterol (15 μL) was used. Separately, a solution of apoA1 protein in PBS (6 mL, 0.1 mg/mL), apoA1-IL4 protein in PBS (6 mL, 0.17 mg/mL) or apoA1-IL4m in PBS (6 mL, 0.18 mg/mL) was prepared.

Both solutions were simultaneously injected using a microfluidic pump fusion 100 (Chemyx Inc) into a Zeonor herringbone mixers (Microfluidic Chipshop, product code: 10000076) with a flow rate of 0.75 mL/minute for the lipid solution and a rate of 6 mL/minute for the apoA1 solution. The obtained solution was concentrated by centrifugal filtration using either a 10 kDa MWCO for discoidal and a 100 kDa MWCO for spherical aNPs Vivaspin tube at 4000 rpm to obtain a volume of 1 mL. PBS (5 mL) was added, and the solution was concentrated to 5 ml; this was repeated twice. The washed solution was concentrated to approximately 1.5 ml and filtered through a 0.22 μm PES syringe filter to obtain the finished aNPs. Protein concentration in aNP samples was quantified with the Pierce™ BCA Protein Assay Kit (Thermo Fisher Scientific). To formulated fluorescent aNPs, 0.5 mg of DiOC18(3) dye (DiO) (Thermo Fisher Scientific) was dissolved in the chloroform solution used to prepare the lipid film.

Determining aNP size and dispersity by DLS: Obtained aNP formulations in PBS were filtered through a 0.22 μm PES syringe filter and analyzed by dynamic light scattering on a Malvern Zetasizer Nano ZS analyzer. Values are reported as the mean number average size distribution.

Radiolabeling aNPs: IL4, apoA1-IL4 and IL4-aNPs were incubated with 2 molar excesses of DFO-p-NCS (5 mg/mL in DMSO) for 2 hours washed three times using a 10 kDa MWCO Vivaspin tubes to remove any unreacted DFO-p-NCS. For radiolabeling, DFO coupled proteins and aNPs were incubated with ⁸⁹Zr at 37° C. using a thermomixer at 600 rpm for 1 hour and washed three times using 10 kDa MWCO Vivaspin tubes to remove any unreacted ⁸⁹Zr.

Cryo-transmission electron microscopy (cryo-TEM) of/L4-aNPso: First, the surface of 200-mesh lacey carbon supported copper grids (Electron Microscopy Sciences) was plasma treated for 40 seconds using a Cressington 208 carbon coater. Subsequently, 3 ml of IL4-aNPs sample (˜1 mg protein/ml) was applied on a grid and vitrified into a thin film by plunge vitrification in liquid ethane by using an automated robot (FEI Vitrobot Mark IV). Cryo-TEM imaging was performed on the cryoTITAN (Thermo Fisher Scientific), equipped with a field emission gun (FEG), a post-column Gatan imaging filter (model 2002) and a post-GIF 2 k×2 k Gatan CCD camera (model 794).The images were acquired at 300 kV acceleration voltage in bright-field TEM mode with zero-loss energy filtering at either 6,500× (dose rate of 1.64 electrons/A²·s) or 24,000× magnification (dose rate of 11.8 electrons/A²·s), and 1s acquisition time.

Super-resolution fluorescence microscopy of IL4-aNPs' interactions with IL4 receptor in human monocytes: Human monocytes were isolated from a healthy donor's peripheral blood as described above. 100,000 monocytes were seeded per well on a cell culture-treated chambered coverslip (μ-Slide 8-well, IBID). After 2 hours of incubation at 37° C. (cell attachment), the cells were incubated for 2 h at 37° C. with Cy5-labeled variant of either bare apoA1 or apoA1-IL4, discoidal aNPs or IL4-aNPs, spherical aNPs or IL4-aNPs. Subsequently, the cells were washed with PBS and fixed with 4% PFA for 20 minutes. IL4 receptor was stained with a polyclonal rabbit IgG1 anti-human IL4R (Thermo Fisher Scientific; 1:100 dilution) primary antibody for 24 hours at 4° C., followed by a goat anti-rabbit Alexa Fluor 488-conjugated secondary antibody (Thermo Fischer Scientific; dilution 1:500) for 1 hour at room temperature. The stained cells were stored in PBS at 4° C. For the direct stochastic optical reconstruction microscopy (dSTORM), the cells were immersed in GLOXY imaging buffer (40 μg/ml catalase, 0.5 mg/ml glucose oxidase, 5% glucose and 0.01 M cysteamine in PBS, pH 8.0) for a few minutes before and during the imaging. The acquisition was performed in Total Internal Reflection Fluorescence (TIRF) mode, using ONI Nanoimager (ONI, Oxford, UK). It is equipped with a 100×/1.4NA oil immersion objective, a sCMOS camera, and, in this study, 488 nm (200 mW) and 640 nm (1000 mW) lasers were used. 10000 frames were acquired with a 10 ms exposure time with the field of view of 50×80 μm. Raw data were processed using the ThunderSTORM software, yielding images with a spatial resolution of 10 nm.

Animal models: Female C57BL/6 mice were purchased from The Jackson Laboratory. For nonhuman primate studies, two male cynomolgus monkeys (Macaca fascicularis) were used. All animals were cohoused in climate-controlled conditions with 12-hour light-dark cycles and provided water ad libitum. Mice were fed a standard chow diet and nonhuman primates were fed Teklad Global 20% Protein Primate Diet. Animal care and experimental procedures were based on approved institutional protocols from the Icahn School of Medicine at Mount Sinai. All mice were randomly assigned to experimental groups.

Pharmacokinetics and biodistribution in mice and non-human primates: C57BL/6 mice were intravenously injected with ⁸⁹Zr-labeled-IL4 variants, respectively, IL4 (53.6±6.6 μCi), apoA1-IL4 (30.1±0.9 μCi), discoidal IL4-aNPs (146.1±46.5 μCi), and spherical IL4-aNPs (108.6±16.9 μCi). Two non-human primates were injected with discoidal ⁸⁹Zr-labeled-IL4-aNPs (1079 μCi and 682 μCi). At predetermined time points, 1, 2, 5, 10, and 30 minutes, and 1, 2, 4, 8, and 24 hours for mice and 5, 30 and 90 minutes and 48 hours for non-human primates, after injection blood was drawn, weighed, and radioactivity was measured using a Wizard² 2480 automatic gamma counter (Perkin Elmer, Waltham, MA). Data was corrected for radioactive decay, percentage of injected dose per gram of blood (% ID/g) was calculated. Data was fitted using a non-linear two-phase decay regression in GraphPad Prism and weighted blood half-life was calculated via the equation (% fast×t_{1/2} fast+% slow×t_{1/2})/100. Biodistribution in mice was determined 24 hours post injection. After PBS perfusion, tissues of interest were harvested, weighed, and radioactivity was measured using a Wizard² 2480 automatic gamma counter (Perkin Elmer, Waltham, MA). Data was corrected for radioactive decay, percentage of injected dose per gram of tissue (% ID/g) was calculated.

PET/CT imaging of aNP biodistribution in mice: C57BL/6 mice were injected intravenously with ⁸⁹Zr-labeled-IL4 variants, respectively, IL4 (53.6±6.6 μCi), apoA1-IL4 (30.1±0.9 μCi), discoidal IL4-aNPs (146.1±46.5 μCi), and spherical IL4-aNPs (108.6±16.9 μCi). After 24 hours, mice were anesthetized using 1.0% isoflurane in O₂ at a flow rate of ˜1.0 liter/minute. PET/CT scans were acquired using a Mediso nanoScan PET/CT (Mediso, Budapest, Hungary). A whole-body CT scan was executed (energy, 50 kVp; current, 180 μAs; isotropic voxel size, 0.25 mm) followed by a 20-minute PET scan. Reconstruction was performed with attenuation correction using the TeraTomo 3D reconstruction algorithm from the Mediso Nucline software. The coincidences were excluded by an energy window between 400 and 600 keV. The voxel size was isotropic with 0.4-mm width, and the reconstruction was applied for four full iterations, six subsets per iteration.

Autoradiography: Tissues were placed in a film cassette against a phosphorimaging plate (BASMS-2325, Fujifilm) at −20° C. to determine the radioactivity distribution. The plates were read at a pixel resolution of 25 mm with a Typhoon 7000IP plate reader (GE Healthcare).

Cellular specificity flow cytometry: For cellular specificity, mice were intravenously injected with DiO labeled IL4-aNPs that was allowed to circulate for 24 hours. Subsequently, mice were sacrificed, and single cell suspensions were created from blood, spleen and bone marrow as previously described. Cell suspensions were incubated with anti-CD115, anti-CD11b, anti-Ly6C, anti-Ly6G, anti-CD19, anti-CD45, anti-CD11c, anti-CD3, anti-F4/80. Live/Dead Aqua was used as viability stain. Cells were subsequently washed and resuspended in FACS-buffer. All data were acquired on an Aurora 5 L flow cytometer (Cytek Biosciences). DiO-IL4-aNPs were detected in the FITC channel.

PET/MRI non-human primate biodistribution: After overnight fasting, non-human primates were anaesthetized using ketamine (5 mg/kg) and dexmedetomidine (0.0075-0.015 mg/kg). Non-human primates were injected with 1.114 mCi and 0.682 mCi discoidal ⁸⁹Zr-labeled-IL4-aNPs, at a dose of approximately 0.1 mg/kg. Dynamic PET imaging as performed for 60 minutes following infusion, and additional static PET/MRI scans were performed at 1 hour and 48 hours after injection. Furthermore, blood was drawn during imaging at 5, 30, and 120 minutes after injection. PET and MRI images were acquired using a 3T PE/MRI system (Biograph mMR, Siemens Healthineers). Beginning concurrently with the injection of aNPs, dynamic PET imaging was performed using one bed position covering the chest and abdomen. MR imaging parameters were as follows: acquisition plane, coronal; repetition time, 1,000 ms; echo time, 79 ms; number of slices, 144; number of averages, 4; spatial resolution of 0.5×0.5×1.0 mm³ and acquisition duration, 42 minutes and 42 seconds. After dynamic PET image acquisition, static whole-body PET images were acquired from the cranium to the pelvis, using 4 consecutive bed positions of 15 min each. Simultaneously with each bed, MR images were acquired as described above, except using only 1.4 signal averages, number of slices 160, and spatial resolution 0.6×0.6×1.0 mm³ (acquisition duration, 14 minutes 56 seconds per bed). Whole-body PET and MR imaging was also performed at 48 hours after injection, using 4 PET bed positions of 30 minutes each, with MR parameters as follows: acquisition plane, coronal; repetition time, 1,000 ms; echo time, 79 ms; number of slices, 224; number of averages, 2; spatial resolution of 0.6×0.6×1.0 mm³; acquisition duration, 29 minutes and 56 seconds. Whole-body MR images from each bed were automatically collated together by the scanner. After acquisition, PET raw data from each bed were reconstructed and collated together offline using the Siemens proprietary e7tools with an Ordered Subset Expectation Maximization (OSEM) algorithm with Point Spread Function (PSF) correction for 3 iterations and 24 subsets. Also, Gaussian filter of 4 mm was applied to the images. A three-compartment (soft tissue, lung and air) attenuation map was used for attenuation.

Imaging-based analysis of the IL4-aNP biodistribution in non-human primates: Image analysis was performed using Osirix MD, version 11.0. Whole-body MR images were fused with PET images and analyzed in a coronal plane. Regions of interest (ROIs) were drawn on tissues of interest including, spleen, liver, kidneys, lungs, heart, cerebellum, and cerebrum were traced in their entirety, and bone marrow uptake was determined using three vertebrae in the lumbar spine. For each ROI, mean standardized uptake values (SUVs) were calculated. Discoidal ⁸⁹Zr-labeled-IL4-aNP uptake per organ was expressed as the average of all mean SUV values per organ.

In vivo tolerance model: For in vivo tolerance model, 11-weeks-old female C57BL/6 mice were intraperitoneal tolerized with 0.1 mg/kg body weight LPS. At 24 and 48 hours, mice were treated intravenously with either 200 μg IL4m-aNPs or PBS. Subsequently, mice were rechallenged with intraperitoneal 0.1 mg/kg LPS injection at 72 hours. After 90 minutes, mice were sacrificed, blood collected for ELISA and single cell suspensions were created from blood, spleen and bone marrow. Staining protocol. Blood samples for ELISA were allowed to clot at RT for 30 minutes. Serum was taken after centrifugation at 1000×g for 10 minutes at 4° C. Mouse TNF and IL6 ELISAs (Biolegend) were performed according to manufacturer's protocols. Animal care and experimental procedures were based on approved institutional protocols from the Nijmegen Animal Experiments Committee.

Statistical analysis: Data are shown as mean+/−SD, unless otherwise indicated. Individual data points in graphs are biological replicates, not technical repeats. Wherever the number of data points cannot be clearly discerned from the figure, n is indicated in the figure legend. Unless otherwise indicated, statistical analyses were performed in Graphpad Prism (V9, Graphpad Software). For trained immunity- and acute stimulation experiments with primary human monocytes, (paired, non-parametric) Wilcoxon signed-rank tests were used. Statistical methods for RNA-sequencing analysis are described above. Two-sided P values under 0.05 were considered statistically significant. Statistical significance in figures is indicated as follows: *=P<0.05, **=P<0.01, ***=P<0.001, NS=P≥0.05.

Data and Code availability; Data are available upon request to the Lead Contact. Raw RNA sequencing data are deposited in the NCBI Gene Expression Omnibus under accession number: GSE185433.

### Results

### IL4 Inhibits Acute Inflammation, Yet Induces Trained Immunity

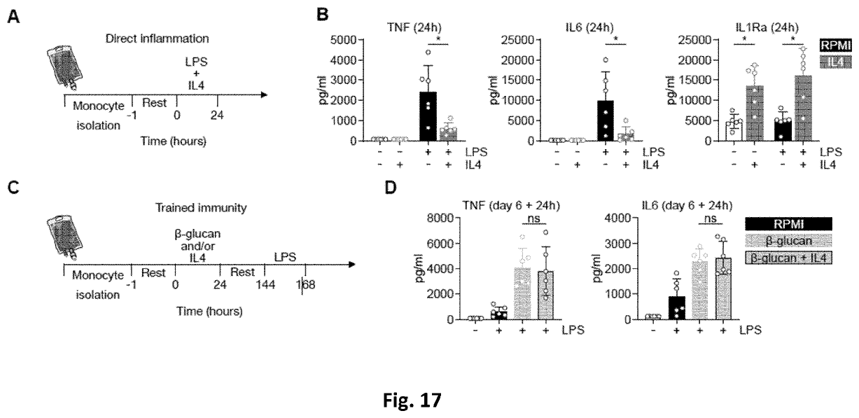

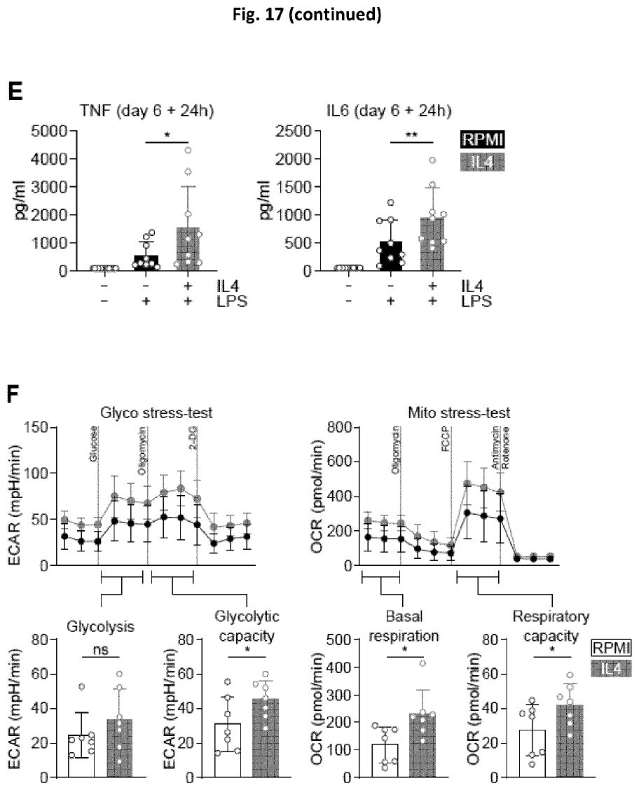

In the context of myeloid cell immunology, IL4 is known primarily for its anti-inflammatory properties. Therefore, present inventors first validated several known inhibitory effects of IL4 on inflammation in primary human monocytes (FIG. 17A). Present inventors stimulated Percoll-enriched monocytes with LPS for 24 hours, in the presence or absence of IL4 (25 ng/mL). As expected, IL4 potently inhibited the secretion of the pro-inflammatory cytokines tumor necrosis factor (TNF) and IL6 (FIG. 17B). Interestingly, IL4-treated cells secreted significantly more IL-1Ra compared to controls (FIG. 17B). As glycolysis is upregulated in activated myeloid cells, present inventors measured lactate production in otherwise unstimulated monocytes treated with IL4 or a medium control. Present inventors found IL4 to slightly, but significantly, lower baseline lactate production (data not shown), confirming its acute anti-inflammatory properties.

Based on these anti-inflammatory properties, present inventors hypothesized that IL4 might also inhibit the induction of trained immunity (FIG. 17C). To test this hypothesis, monocytes were trained with β-glucan, a prototypical trained immunity stimulus, for 24 hours, followed by washing the stimulus and a 5-day resting period in culture medium. On day 6, present inventors re-stimulated the cells with LPS for another 24 hours and measured TNF and IL6 (FIG. 17D). While β-glucan induced trained immunity as expected, addition of IL4 in the first 24 hours did not inhibit the training effect (FIG. 17D). Contrary to present inventors' initial hypothesis, exposing monocytes to IL4 alone for 24 hours induced a trained immunity phenotype on day 6 (FIG. 17E). Besides enhanced production of pro-inflammatory cytokines, IL4-trained cells produced more lactate at baseline (data not shown). IL4-trained cells were slightly less effective at phagocytosing heat-killed Candida albicans than untrained controls (data not shown). Collectively, the data of present inventors demonstrate that IL4 inhibits inflammation and induces trained immunity, at both metabolic and functional immunologic level.

Encouraged by these observations, present inventors comprehensively studied the metabolic alterations following IL4-induced trained immunity. To this aim, present inventors employed Seahorse metabolic flux analyses to probe glycolytic and oxidative metabolism of IL4-trained cells and unstimulated controls. IL4 training on day 0 had a marked effect on metabolic parameters measured on day 6 (FIG. 17F), with a trend towards higher basal glycolysis, and a significant increase of oligomycin-triggered maximum glycolytic capacity (FIG. 17F left panel). In addition, both baseline- and FCCP-triggered maximum respiration rates were significantly augmented by IL4 training (FIG. 17F right panel).

Present inventors then used flow cytometry to measure several parameters commonly associated with IL4 activation of monocytes/macrophages (data not shown). IL4 training caused a strong downregulation of CD14 expression on day 6. In contrast, CD200R and especially CD206 were significantly enhanced on day 6 subsequent to IL4-activation on day 0. CD80 was marginally increased by IL4 training, but overall expression was still low on these otherwise naive macrophages. It is known that monocyte-derived dendritic cells (moDCs, which are differentiated using IL4+GM-CSF) also downregulate CD14, whilst strongly upregulating CD1c. IL4-trained cells expressed slightly more CD1c than untrained cells, but far less than moDCs (data not shown). These results indicate IL4 induces a program of trained immunity which incorporates features known from classical IL4 immunological functions.

### Immune and Epigenetic Mechanisms Mediating IL4-Induced Trained Immunity

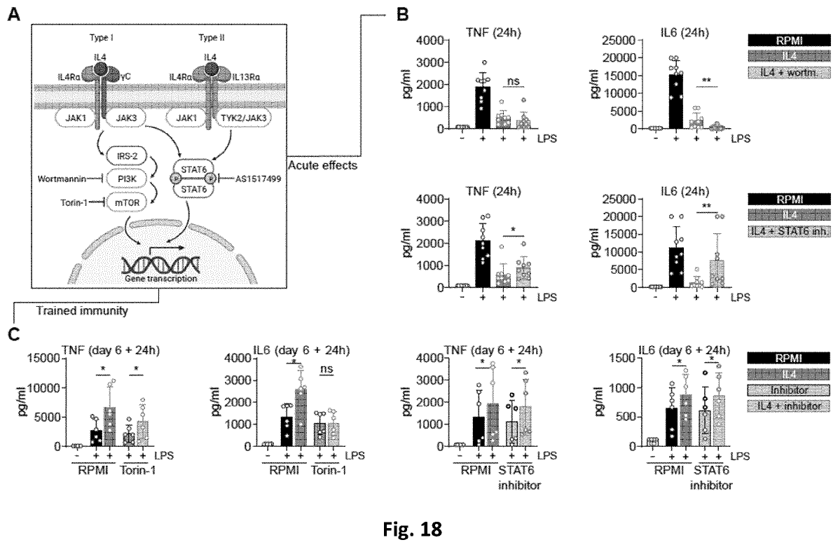

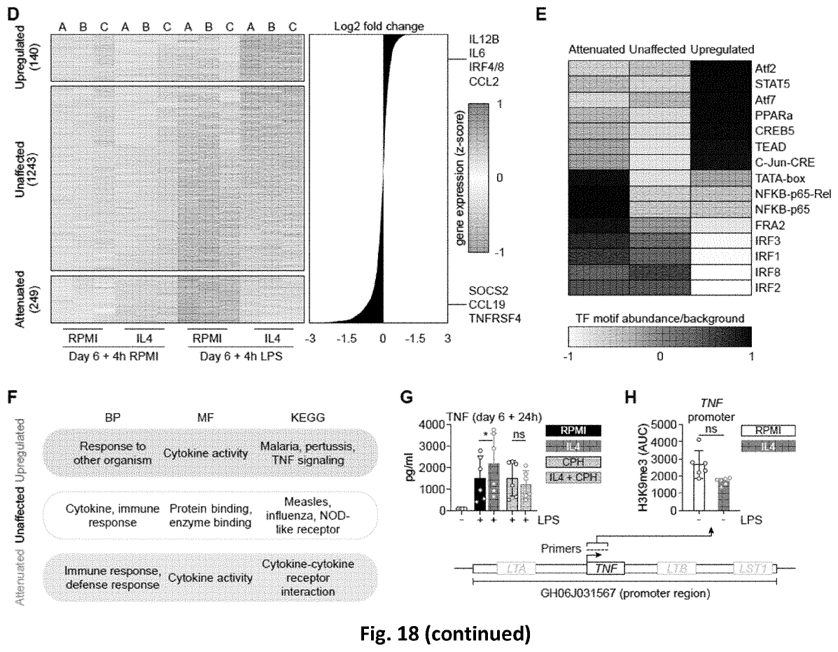

The signaling mechanisms of IL4 are well-described: the IRS-2/PI3K/mTOR axis and the STAT6 signaling pathway (FIG. 18A). Present inventors performed pharmacological inhibition experiments to investigate the role of these pathways for both inhibition of acute inflammation as well as trained immunity induction by IL4. Inhibition of PI3K or mTOR (using wortmannin or torin-1, respectively) did not abrogate the effect of IL4 on acute inflammation, but diminished the trained immunity responses (FIG. 18B, 18C and data not shown). IL4 training, as measured by an increased TNF and IL6 production, was significantly blunted in the presence of torin-1 (FIG. 18C and data not shown). In contrast, the STAT6 inhibitor AS1517499 partly restored cytokine production in acute inflammatory responses, but did not affect trained immunity induction by IL4 (FIGS. 18B and 18C). Thus, each of the signaling pathways induced downstream of IL4 engagement with its receptors has distinct functions: IL4 exerts its known acute anti-inflammatory function through STAT6, but simultaneously induces trained immunity via PI3K/mTOR, a previously unknown pro-inflammatory effect.

To gain insight into the molecular program induced by IL4 training, present inventors performed transcriptomics analysis on naïve and IL4-trained macrophages, both before and after LPS re-stimulation on day 6. Overall, 140 genes were more strongly induced (‘upregulated’) in IL4-trained macrophages, whereas 249 genes were attenuated (FIG. 18D). Amongst the top upregulated genes were pro-inflammatory cytokines such as IL6 and IL12B, that are known to be involved in trained immunity. Among the prominent attenuated genes were CCL19 and SOCS2, which are important for lymphocyte trafficking and suppression of cytokine signaling, respectively.

Present inventors next performed transcription factor (TF) motif enrichment analysis (FIG. 18E) and gene ontology/pathway enrichment analyses (FIG. 18F) to gain further insight into the transcriptome profiles. Promoters of genes upregulated in IL4-trained macrophages were highly enriched for motifs recognized by TFs such as ATF2/ATF7, PPARα, and STAT5, whereas interferon regulatory factor (IRF) motifs were especially depleted. This pattern was mostly reversed for unaffected- and attenuated genes, except for TATA-box, NFκB-p65, NFκB-p65-Rel, and FRA2: these motifs were highly enriched in attenuated gene promoters, but decreased in both unaffected- and upregulated genes (FIG. 18E). Gene ontology (biological process; BP, and molecular function; MF) and KEGG pathway enrichment showed that immunological activities were present in both upregulated (e.g. BP “Response to organism”, KEGG “TNF signaling”) and attenuated (e.g. BP “Immune response”, MF “Cytokine activity”) gene sets (FIG. 18F). Present inventors performed a similar transcriptome analysis on monocytes stimulated immediately after isolation with IL4, LPS, or IL4 and LPS combined, which confirmed an acute anti-inflammatory transcriptomic response to IL4 (data not shown). Together, these data reveal specific transcriptional programs in both the acute anti-inflammatory effects and the long-term trained immunity responses invoked by IL4.

Present inventors subsequently investigated the importance and presence of epigenetic reprogramming, specifically histone 1 modifications. Addition of the anti-allergy drug cyproheptadine, a SET7 (also known as SET9) histone 2 methyltransferase inhibitor, abrogated the induction of trained immunity by IL4 (FIG. 18G). SET7 has been described earlier as an important epigenetic mediator of trained immunity22. Furthermore, present inventors evaluated H3K9me3-mediated repression of TNF using chromatin-immunoprecipitation (ChIP)-qPCR analyses in IL4 induced trained immunity. Using an area under the curve (AUC) analysis of primer pairs showed a decrease of H3K9me3 6 in IL4-induced trained immunity, although this did not reach statistical significance (FIG. 18H and data not shown). Together, these data indicate epigenetic reprogramming is crucial for, and characteristic of, IL4-induced trained immunity.

Developing an apoA1-IL4 Fusion Protein that Integrates in Lipid Nanoparticles

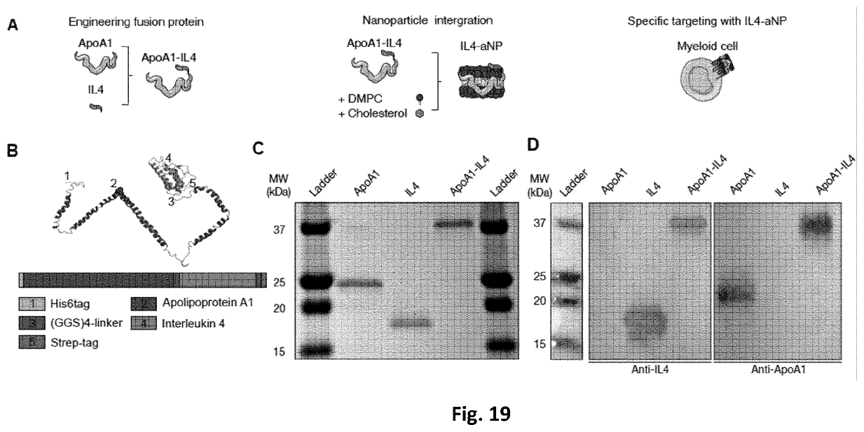

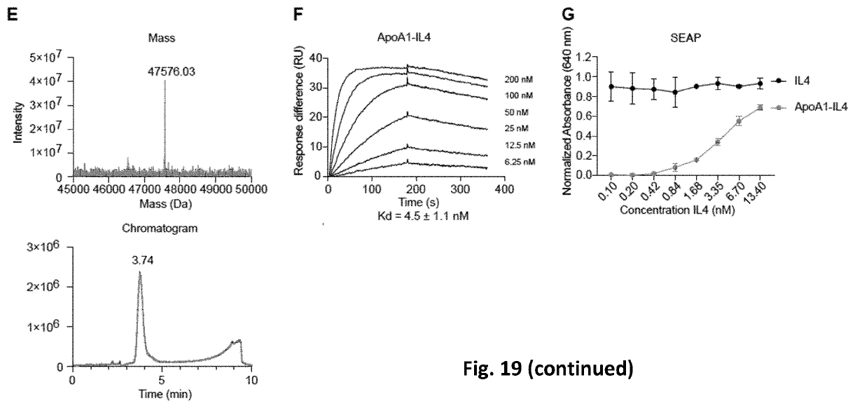

Despite its unique ability to inhibit acute inflammation while simultaneously inducing trained immunity, recombinant IL4's clinical translation is hampered by its unfavorable pharmacokinetic properties. To overcome this limitation, present inventors developed an apoA1-based fusion protein that readily integrates in lipid nanoparticles to yield IL4-containing nanoparticles (IL4-aNPs). ApoA1-based nanoparticles (aNPs) inherently accumulate in hematopoietic organs and efficiently target myeloid cells and their progenitors (Schrijver, D. P. et al. in Advanced Therapeutics Vol. 4 2100083-2100083 (John Wiley & Sons, Ltd, 2021; van Leent, M. M. T. et al. Regulating trained immunity with nanomedicine. Nature Reviews Materials 7, 46 465-481, doi:10.1038/s41578-021-00413-w (2022)) (FIG. 19A). Specifically, present inventors designed a fusion protein consisting of human apoA1 and human IL4 (apoA1-IL4) connected via a flexible linker and flanked by two purification tags, a 6his-tag located at the N-terminus and a strep-tag at the C-terminus (FIG. 19B). Present inventors used molecular characterization techniques to confirm the nature and purity of apoA1-IL4. By performing sodium dodecyl sulfate-polyacrylamide gel electrophoresis (SDS-PAGE) on each purified protein sample, present inventors confirmed the presence of protein with molecular weights of 25 kDa (apoA1), 18 kDa (IL4) and 37 kDa (apoA1-IL4) (FIG. 19C), while Western-blots indicated the presence of apoA1 and IL4 (FIG. 19D). Due to its amphophilic properties, proteins consisting of apoA1 run lower on the gel than expected. These observations were corroborated by quadrupole time-of-flight (Q-TOF) mass spectrometry showing a single mass peak at 47576.03 Da corresponding to apoA1's expected molecular weight of 47582.57 Da (FIG. 19E).

Before integrating apoA1-IL4 in lipid nanoparticles, biophysical and cellular analysis—using surface plasmon resonance (SPR) and HEK-Blue™ IL4/IL13 (HEK-IL4) reporter cells—were performed to determine the preservation of biological activity after the purification and refolding process. Present inventors determined the equilibrium dissociation constants Kd of apoA1-IL4 against the human IL4 receptor alpha (IL4Rα) using SPR to be 4.5 nM±1.1 nM (FIG. 19F). HEK-IL4 cells possess an IL4Rα/STAT6-inducible reporter gene coding for secreted alkaline phosphatase (SEAP), which they prominently produced upon introduction of apoA1-IL4 to their culture wells. This indicates biological activity of apoA1-IL4 (FIG. 19G). Although fusion with apoA1 significantly altered IL4's biophysical properties, enabling integration into lipid nanoparticles, binding to its receptor was preserved with a kd of 0.28±0.1 nM (data not shown). To summarize, present inventors have developed an apoA1-IL4 fusion protein that preserves IL4's biological activities after extraction, purification, and refolding, and contains the desirable physicochemical features for its integration into lipid nanoparticles through apoA1.

Integrating apoA1-IL4 in Lipid Nanoparticles

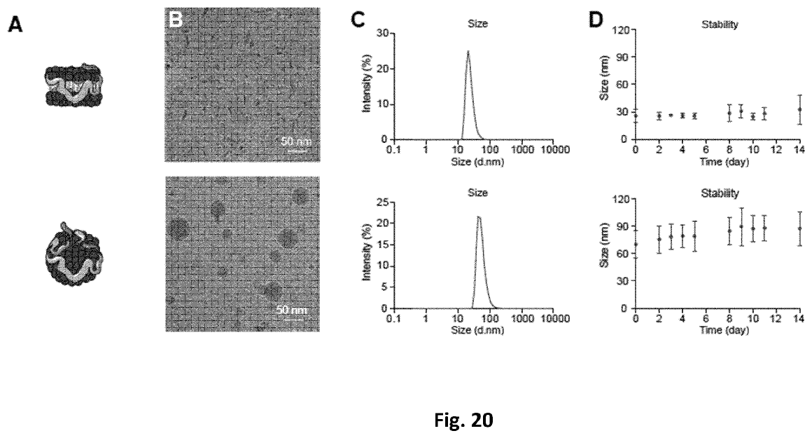

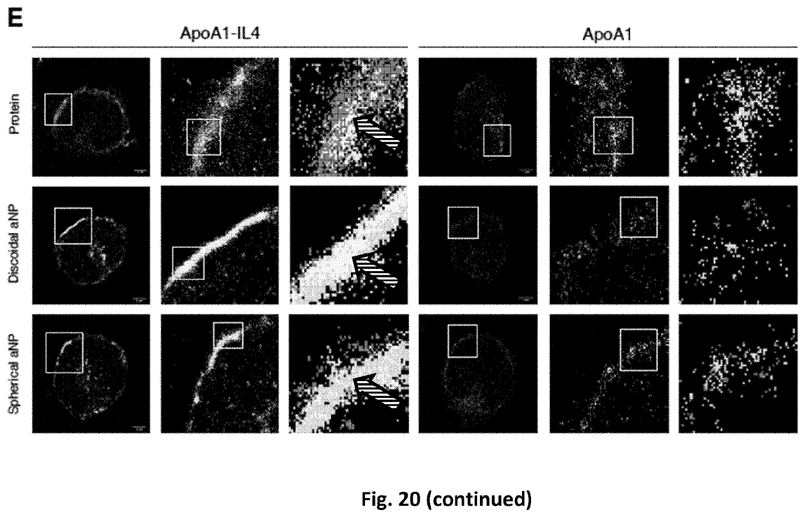

In order to improve IL4's pharmacokinetic properties and bioavailability to myeloid cells, present inventors integrated the apoA1-IL4 fusion protein in lipid nanoparticles to yield IL4-aNPs. Nanoparticles of different size and morphology were obtained by varying the nanomaterials' compositions (FIG. 20A). The successful formulation of discoidal and spherical nanoparticles was confirmed by cryogenic transmission electron microscopy (cryo-TEM) (FIG. 20B). Present inventors additionally analyzed nanoparticle size and stability in PBS for 14 days using dynamic light scattering (DLS) (FIGS. 20C and 20D). IL4-aNPs remain stable for 14 days, and have a similar size and stability compared to present inventors' conventional aNPs (data not shown). Conventional aNPs comprise apolipoproteins such as described in Schrijver, D. P. et al. in Advanced Therapeutics Vol. 4 2100083-2100083).

Next, present inventors investigated the interaction of IL4-aNPs with primary human monocytes. Direct stochastic optical reconstruction microscopy (dSTORM) analysis revealed the expression of IL4Rα on the membrane. Furthermore, binding of bare apoA1, bare apoA1-IL4, and IL4-aNPs were confirmed by the rim covering the cell surface. When focusing on a zoomed in section of the membrane, present inventors found that bare apoA1-IL4 and IL4-aNPs were associated with the IL4Rα, forming enriched co-clusters on the cell surface, which we did not observe for bare apoA1 and conventional aNPs (FIG. 20E). Together, present inventors' DLS size stability assays, cryoTEM, and dSTORM analyses revealed that the integrating the apoA1-IL4 fusion protein in lipid nanoparticles yields biologically functional IL4-aNPs.

Studying IL4, apoA1-IL4 and IL4-aNP Formulations in Mice

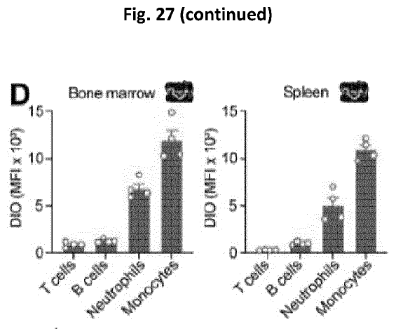

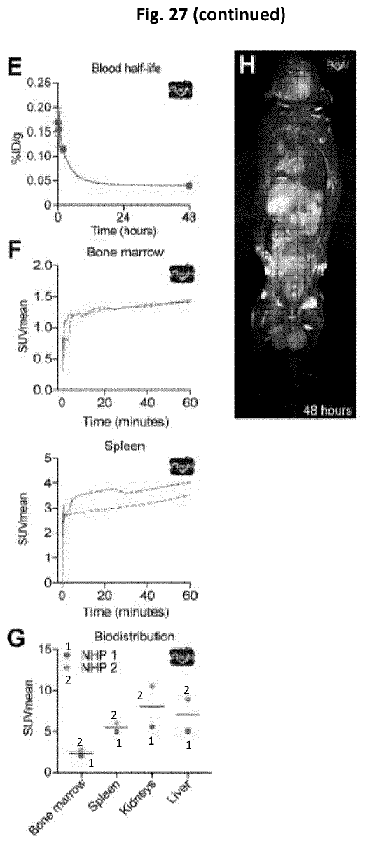

To investigate pharmacokinetics and biodistribution in C57BL/6 mice, present inventors radiolabeled the protein component of four different IL4 therapeutics, namely recombinant IL4; the bare apoA1-IL4 fusion protein; as well as discoidal and spherical IL4-aNPs, with zirconium-89 (⁸⁹Zr). Note that these experiments were performed with human variant of IL4 that do not display biologic activity in mice. Positron emission tomography with computed tomography (PET/CT) imaging at 24 hours post intravenous administration showed that ⁸⁹Zr-IL4 and ⁸⁹Zr-apoA1-IL4 accumulated mostly in the kidney and liver. In contrast, besides accumulating in the liver and kidney, ⁸⁹Zr-IL4-aNPs accumulated in relatively higher amounts in immune cell-rich organs, including the spleen and bone marrow (FIG. 27A). Present inventors performed ex-vivo gamma counting to determine their nanomaterials' blood half-lives and uptake in major organs (FIGS. 27B and 27C), which present inventors corroborated by autoradiography (data not shown). Comparison of uptake ratios by target organs (bone marrow+spleen) divided by clearance organs (kidney+liver) showed a significant increase in uptake ratio for IL4-aNP formulations compared to unformulated fusion protein and bare IL4 (data not shown). Next, present inventors used flow cytometry to measure cell type-specific biodistribution in target organs. DiO-labeled discoidal IL4-aNPs accumulate in myeloid cells, most notably monocytes and neutrophils, in both spleen and bone marrow, while they do not (or only marginally) interact with lymphocytes (FIG. 27D). Based on its favorable (and myeloid-specific) uptake in hematopoietic organs, present inventors selected the discoidal IL4-aNP formulation for further studies in non-human primates and translational models of inflammation and sepsis.

### IL4-aNP Immunotherapy Displays Favorable Uptake Profile in Non-Human Primates

To evaluate the clinical translatability of IL4-aNP immunotherapy, present inventors determined their biodistribution and safety in non-human primates. Two non-human primates were injected intravenously with ⁸⁹Zr-IL4-aNPs. Their in vivo behavior was studied in vivo using fully integrated three-dimensional PET combined with magnetic resonance imaging (PET/MRI). After injection, dynamic PET/MRI (data not shown) demonstrated rapid accumulation of IL4-aNPs in the liver, kidney (data not shown), spleen and bone marrow (FIG. 27E-H). In accordance with present inventors' mouse data, no undesirable uptake of IL4-aNPs was observed in non-target organs including the brain and heart (data not shown). Together, these results that demonstrate IL4-aNP's favorable biodistribution and safety profile is retained across species, corroborating this immunotherapy's translational potential.

### IL4-aNP Therapy Resolves Immunoparalysis In Vitro and In Vivo

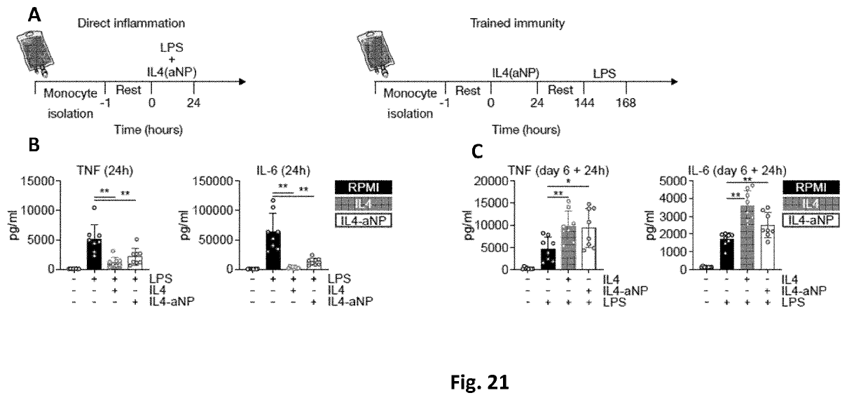

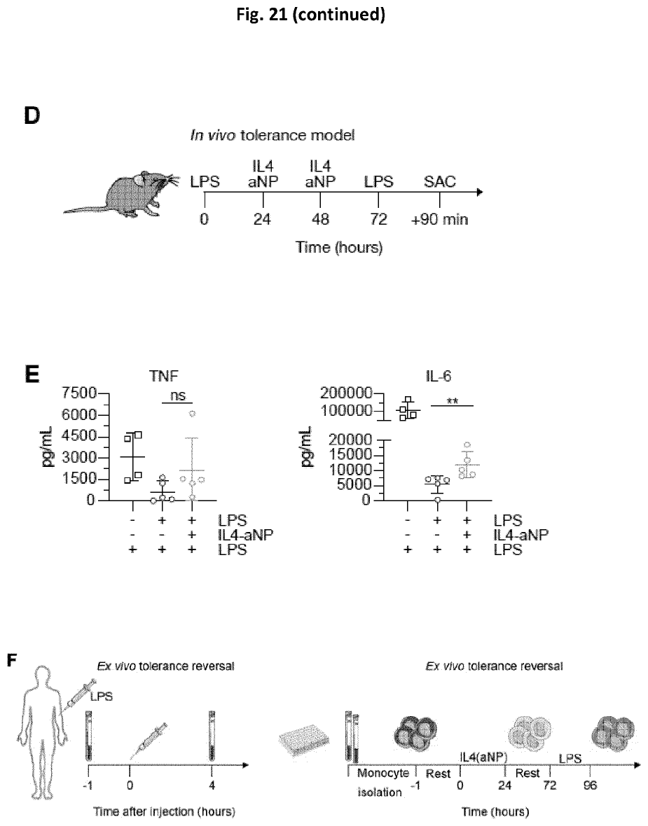

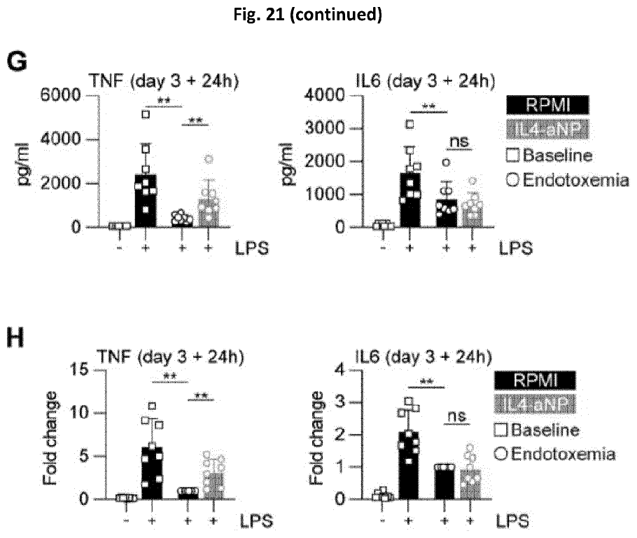

After establishing that natural IL4 simultaneously dampens acute inflammatory responses and induces a program of trained immunity, present inventors assessed IL4-aNPs' effects on monocytes in vitro (FIG. 21A). Present inventors based the dose for in vitro experiments on efficiency of phospho-STAT6 induction in primary human monocytes, relative to bare IL4 (data not shown). Indeed, IL4-aNPs (molar equivalent of 200 ng/mL bare IL4) significantly reduced TNF and IL6 production of LPS-stimulated monocytes (FIG. 21B), while enhancing long-term responsiveness of monocytes on day 6 (FIG. 21C). These data indicate that IL4-aNPs, similarly to IL4, suppress acute inflammation and induce trained immunity in vitro. While present inventors' in vivo biodistribution data shows that IL4-aNPs specifically target myeloid cells (FIG. 27D), IL4-induced trained immunity changes surface marker expression of macrophages, which are antigen-presenting cells (data not shown). This in turn might affect polarization signals during T cell activation. In order to investigate these potential indirect effects of IL4-aNPs on T cells, allogenic naïve T cells were cultured in the presence of IL4-trained macrophages. In this model, HLA-mismatch causes antigen-unspecific T cell activation and polarization. No significant differences in the abundance of T cell subtypes, Th1 (CD4+IFNγHigh+), Th2 (CD4+IL4+), Treg (CD4+IL10+), Th17 (CD4+IL17+) and cytotoxic T cells (CD8+Granzyme B+Perforin+) were observed between trained macrophages and controls (data not shown). Together, these findings indicate that IL4 training does not exhibit the ability to skew T cells responses indirectly, suggesting a predominantly myeloid-specific effect.

Septic patients may experience both hyperinflammatory responses and immunoparalysis, creating a therapeutic paradox. Induction of trained immunity can theoretically be used to reverse immune tolerance, but this has not been translated to in vivo models. One reason is that human IL4 does not display biologic activity in mice. Present inventors therefore designed and produced a chimeric fusion protein, consisting of human apoA1 and murine IL4 for formulation with lipids to yield IL4m-aNPs. Here, present inventors investigated if IL4m-aNPs can reverse LPS-induced tolerance in mice. To that end, present inventors intraperitoneally injected C57B/6 mice with LPS (0.1 mg/kg) to induce immunoparalysis or PBS (as a control). Present inventors intravenously administered IL4m-aNPs (200 μg per dose) at 24 and 48 hours after LPS treatment. Present inventors re-challenged the mice with another intraperitoneal injection of LPS (0.1 mg/kg) 72 hours after the first challenge (FIG. 21D). Indeed, treatment with IL4m-aNPs improved innate immune responses as signified by significantly (p=0.0079) increased serum IL6 concentrations following LPS re-challenge of tolerized mice (FIG. 21E). While TNF levels in some mice were clearly elevated, statistical significance was not achieved (p=0.1508) due to heterogeneity in the therapeutic response (FIG. 21E). Collectively, present inventors' in vitro and in vivo data demonstrate that IL4-aNPs can reduce tolerance.

### Human Endotoxemia Model

After present inventors observed tolerance-reversal in their mouse model, present inventors substantiated these results with a model that more closely mimics human clinical immunoparalysis. Present inventors obtained blood from healthy individuals undergoing experimental human endotoxemia, a standardized controlled model of systemic inflammation capturing hallmarks of both hyperinflammatory and immunoparalytic phenotypes of sepsis (FIG. 21F). In this controlled human model, LPS is intravenously administered to healthy volunteers, leading to a systemic inflammatory response which is followed by tolerization of circulating monocytes, a phenomenon also observed in sepsis-induced immunoparalysis. Blood was collected before and 4 hours after start of LPS administration. Monocytes isolated after LPS administration showed deficient cytokine production upon immediate re-exposure to LPS, indicating tolerization (data not shown). When the tolerant monocytes from the LPS-challenged volunteers were exposed ex vivo to either IL4 or IL4-aNPs for 24 hours, they showed significantly improved production of TNF, but not IL6, upon re-stimulation with LPS on day 3 (FIG. 21G (concentration), 21H (fold change), and data not shown). In contrast, untreated monocytes remained completely tolerant. Together, these data highlight the ability of IL4 and IL4-aNPs to at least partially reverse LPS-tolerance ex vivo.

### Discussion

IL4 is generally considered to be an anti-inflammatory cytokine, while its long-term effects on monocyte/macrophage function are unclear. Present inventors initially expected IL4 would inhibit trained immunity, similarly to IL37 and IL38. Surprisingly, in addition to its known inhibitory effects on acute inflammation, present inventors observed that IL4 induces trained immunity as assessed by increased cytokine production responsiveness. While the induction of trained immunity by IL4 was unexpected, present inventors' observations are in line with IL4's activation of the mTOR 5 signaling cascade, a central mechanism in trained immunity. Present inventors subsequently investigated the long-term effect of IL4 pre-exposure, as well as the capacity of IL4 to reverse immune tolerance induced by experimental endotoxemia. In this context, the results demonstrate that an anti-inflammatory STAT6-dependent cellular program dominates during the acute exposure of cells to IL4, but that this shifts over time towards an mTOR-driven program of long-term trained immunity. IL4-training fits all parameters that are typically used to describe trained immunity, including enhanced cytokine production, epigenetic rewiring, increased metabolic activity, and altered transcriptomic responses upon re-stimulation. These observations are in line with the growing evidence for a dynamic- and timing-dependent model of monocyte differentiation, such as the one proposed in 2017 by Sander et al. (Sander, J. et al. Cellular Differentiation of Human Monocytes Is Regulated by Time-Dependent 7 Interleukin-4 Signaling and the Transcriptional Regulator NCOR2. Immunity 47, 1051-1066 e1012, 8 doi:10.1016/j.immuni.2017.11.024 (2017))

IL4's unique ability to simultaneously suppress acute inflammation while inducing a trained immunity program, which has been reported to improve host defense, can be potentially used to treat severe infections. For example, both sepsis and COVID-19 are characterized by a dysregulated immune response, creating a therapeutic paradox that requires both managing hyperinflammatory responses and improving host defense responses against (opportunistic) secondary infections. In order to take advantage of IL4's unique features, present inventors developed a nanoparticle protein engineering strategy, thereby overcoming this cytokine's unfavorable in vivo pharmacokinetic properties. Present inventors demonstrated that their aNP strategy favorably alters IL4's blood half-life and biodistribution profile, resulting in elevated, neutrophil- and monocyte-specific accumulation in myeloid cell-rich organs such as bone marrow and spleen, as earlier observed in models of trained immunity. Whereas biodistribution was studied with the human IL4-aNPs, Present inventors further developed the murine variant IL4m-aNP to evaluate in vivo efficacy. Indeed, present inventors demonstrated IL4-nanoparticles' immunoparalysis-reverting effect in a LPS-induced cytokine storm mouse model. While the data demonstrate restored innate immune responses, with a significantly increased in IL6 levels and a clear trend towards increased TNF concentrations in serum of mice treated with IL4m-aNP, full blown dose range finding studies are required to expand the potential of IL4-nanoparticle therapy in a range of immune-mediated diseases that are characterized by concurrent hyperinflammation and immunoparalysis. Encouragingly, using the human fusion protein, present inventors showed that IL4-aNPs can reverse immune tolerance of cells acquired from a human model mimicking clinical immunoparalysis.

Present inventors anticipate that their cytokine-nanoparticle platform may find use in immunoparalysis after sepsis induced hyperinflammation, as well as other myeloid-directed applications. Immuno-oncological applications might be considered since cancer is also characterized by local pro-tumor inflammation and simultaneous suppression of anti-tumor responses (often mediated by myeloid cells). Myocardial infarction and stroke are also characterized by sterile inflammation followed by immunoparalysis, and severe trauma patients suffer from a similar immune-paralytic condition. In all these situations, reducing inflammation and overcoming immunoparalysis may be beneficial for patient recovery and preventing secondary infections. Present inventors' IL4-aNP platform harbors the potential to develop into a pivotal therapeutic modality for treatment of all these conditions.

### Example 3. Fusion Proteins of an Apolipoprotein Fused to a Rerouting Molecule and Incorporation Thereof into Lipid Nanoparticles

### Materials and Methods

Expression and purification of VHHCD8-apoA1 fusion protein: A small culture of ClearColi cells transformed with pET20b-VHHCD8-apoA1 plasmid and pDiscoTune plasmid was started in LB medium with 100 μg/mL ampicillin. The next day, 40 mL of small culture was diluted in 1 liter of 2YT medium to start large cultures and rhamnose was added at a final concentration of 50 μM to induce T7 lysozyme on the pDiscoTune plasmid. The culture was grown at 37° C. and 150 rpm until an OD600 of 0.6-0.8, then isopropyl β-d-1-thiogalactopyranoside (IPTG) was added at a final concentration of 0.1 mM to induce expression. The induced culture was incubated overnight at 18° C. and 150 rpm. Induced bacterial cultures were pelleted and cells were resuspended in lysis buffer (20 mM Tris, 500 mM NaCl, pH 7.9). Benzonase Nuclease (Merck Millipore) and one cOmplete™ EDTA-free Protease Inhibitor Cocktail tablet (Roche) per 50 mL cell suspension was added and the cell suspension was incubated at 4° C. while stirring. The suspension was subsequently homogenized three times at 15000-20000 psi using the Avestin Emulsiflex C3. The cell lysate was kept on ice at all times. After lysis, cell lysate was centrifuged to pellet insoluble cell debris and supernatant was flown through an Immobilized Metal Chelate Affinity Chromatography (IMAC) column containing immobilized nickel ions. The column was washed with 8 column volumes of buffer A (20 mM Tris, 500 mM NaCl, 10 mM imidazole, pH 7.9), then 8 column volumes of buffer A50 (20 mM Tris, 500 mM NaCl, 50 mM imidazole, pH 7.9). To elute VHHCD8-apoA1, 8 column volumes of buffer A500 (20 mM Tris, 500 mM NaCl, 500 mM imidazole, pH 7.9) was applied to the column. All fractions of the purification steps were collected and analyzed with SDS-PAGE. The buffer of fractions containing purified VHHCD8-apoA1 was changed to PBS using Amicon Ultracentrifugal Filters (Amicon). To store VHHCD8-apoA1, aliquots were snap-frozen in liquid nitrogen and stored at −70° C.

The VHHCD8-apoA1 fusion protein has a sequence as defined by SEQ ID NO: 54 or is encoded by a sequence as defined by SEQ ID NO: 55, which comprises a linker between apoA1 and VHHCD8 comprising a cysteine.

SDS-page: performed as described in Example 2.

aNP formulation: performed as described in section “formulating nanoparticles” of Example 2.

DLS: performed as described in Example 2.

Cryo-TEM: performed as described in Example 2.

In vitro binding of VHHCD8-apoA1 in mice splenocytes: Spleens were obtained from mice, cut into pieces, and strained through a 70 μm strainer (Corning) multiple times to obtain a splenocyte suspension. Cells were spun down at 1500 rpm for 10 minutes, the supernatant was removed and the cells were dissolved in 2 mL 1× red blood cell lysis buffer (Thermofisher). Suspension was incubated at room temperature for 5 minutes, 10 mL Roswell Park Memorial Institute (RPMI) medium (Thermofisher) was added, and cells were again spun down at 1500 rpm for 10 minutes. Cells were then redissolved in RPMI medium and plated in a 96 wells plate at 150.000 cells/well.

The proteins were labeled by adding sulfo-cyanine5-maleimide (Lumiprobe) in dimethyl sulfoxide (DMSO) at a 5× molar excess. This mixture was incubated at room temperature for 2 hours. Excess dye was removed using a PD minitrap G-25 desalting column (Cytiva). Fluorescently labeled aNPs were formulated by adding 6.4 μg Dil for discoidal formulations and 21 μg Dil for spherical formulations (see section “formulating nanoparticles” of Example 2). Fluorescently labeled fusion proteins or aNPs and controls were added to the wells and incubated for 30 minutes at 4° C. (for proteins) or 37° C. (for aNPs) after which cells were harvested, washed, and stained for CD3 and CD4 and measured on Cytoflex (Beckman Coulter Inc.). Flow cytometry data was analyzed using FlowJo software (BD).

### Results

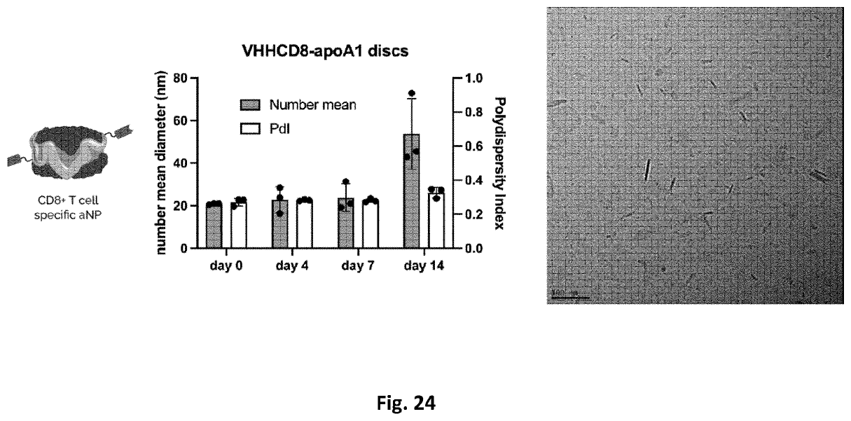

The VHHCD8-apoA1 fusion protein was successfully expressed in Clearcoli cells. Minor protein contaminants were present after IMAC purification [lane E1] (FIG. 23). The most prominent band corresponds to the fusion protein with a molecular weight of 43.3 kDa (FIG. 23). The correct mass was later confirmed via mass spectrometry (data not shown). Discoidal apolipoprotein nanoparticles were formulated incorporating VHHCD8-apoA1. Using Dynamic Light Scattering (DLS), the particles' size and poly dispersity index (PDI) were determined. The size of the particles remained stable for 7 days (FIG. 24 (left panel)). On day 14 the size had increased slightly (FIG. 24 (left panel)). The PDI remained stable for 14 days (FIG. 24 (left panel)). Cryo-TEM images of the nanoparticles showed the expected discoidal shape (FIG. 24 (right panel))

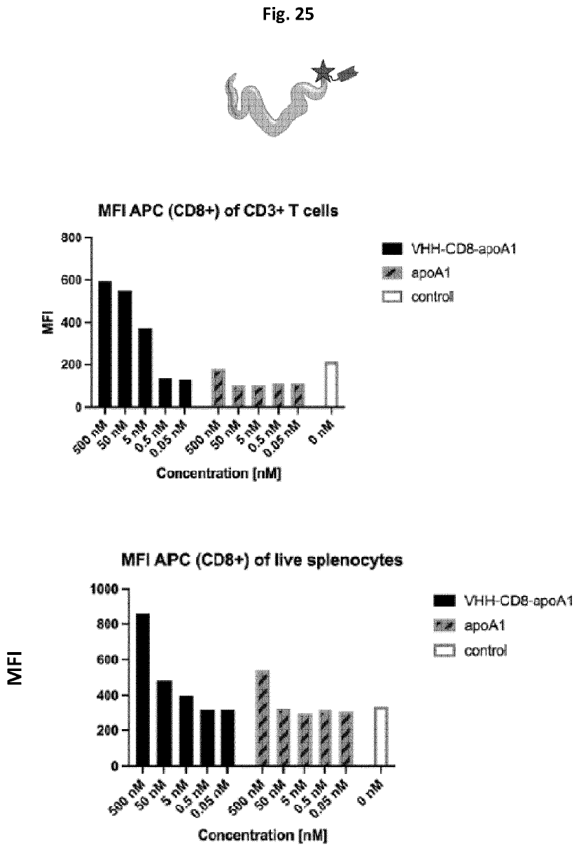

VHHCD8-apoA1 and apoA1 were fluorescently labeled, and subsequently added to mouse splenocytes. For the VHHCD8-apoA1 fusion protein, a dose dependent increase of Mean Fluorescence Intensity (MFI) was observed, indicating the binding of the fusion protein to the CD8 receptor (FIG. 25; lower panel). The apoA1 condition did not show this dose dependent behavior and had a similar MFI to the control sample.

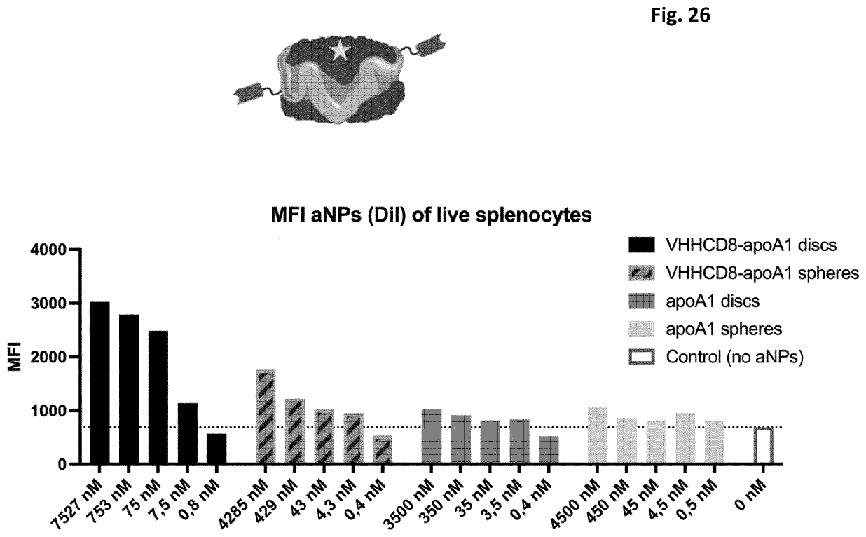

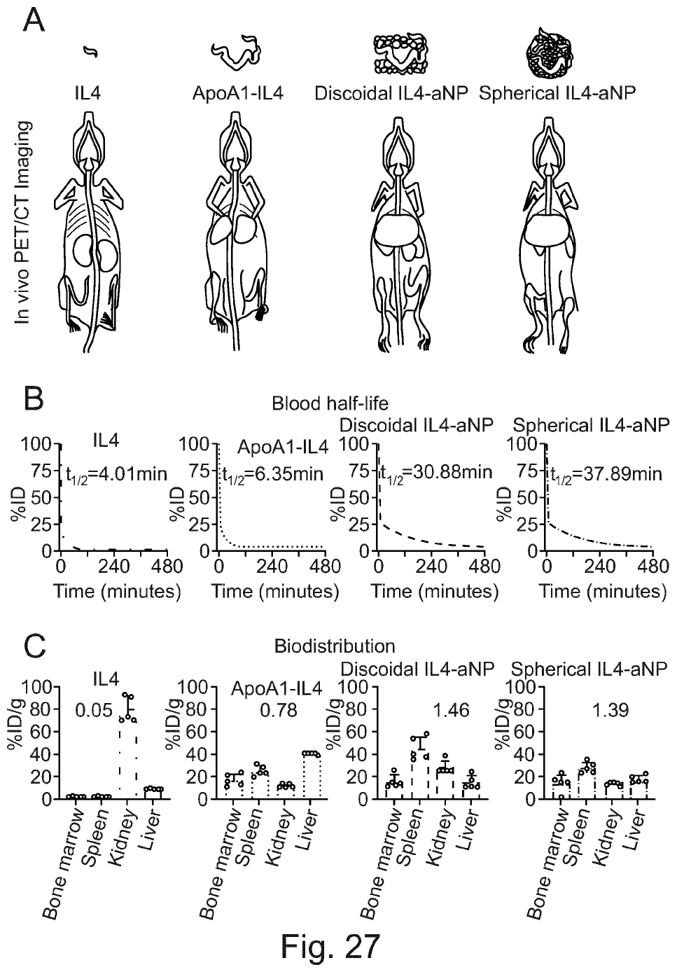

Discoidal and spherical aNPs were formulated with VHHCD8-apoA1 and apoA1. A fluorescent dye was integrated in the lipid structure of the particle. Mouse splenocytes were incubated with the nanoparticles. Both VHHCD8-apoA1 particles showed a dose dependent increase of MFI, indicating the binding of the nanoparticle to the CD8 receptor (FIG. 26). The discoidal nanoparticles showed a greater increase than the spherical nanoparticles. No increase in MFI was observed for the apoA1 nanoparticle conditions.

### SEQUENCE LISTING

The patent application is filed with corresponding sequence listing, which is incorporated in its entirety by reference. Below is an overview of the sequences and their brief description

| SEQ ID | | |
|---|---|---|
| NO: | Type: | Description: |
| 1 | Peptide | Human ApoA1 |
| 2 | Polynucleotide | Human ApoA1 |
| 3 | Peptide | ApoA 1 |
| 4 | Polynucleotide | ApoA 1 |
| 5 | Peptide | His tagged ApoA1 |
| 6 | Polynucleotide | His tagged ApoA1 |
| 7 | Peptide | ApoA1 S14C mutant |
| 8 | Polynucleotide | ApoA1 S14C mutant |
| 9 | Peptide | ApoA1 S157C mutant |
| 10 | Polynucleotide | ApoA1 S157C mutant |
| 11 | Peptide | ApoA1 S239C mutant |
| 12 | Polynucleotide | ApoA1 S239C mutant |
| 13 | Peptide | ApoE |
| 14 | Polynucleotide | ApoE |
| 15 | Peptide | His tagged ApoE |
| 16 | Polynucleotide | His tagged ApoE |
| 17 | Peptide | ApoE S10C mutant |
| 18 | Polynucleotide | ApoE S10C mutant |
| 19 | Peptide | ApoE A292C mutant |
| 20 | Polynucleotide | ApoE A292C mutant |
| 21 | Peptide | ApoA1-IL-1β fusion |
| 22 | Polynucleotide | ApoA1-IL-1β fusion |
| 23 | Peptide | ApoA1-IL2 fusion |
| 24 | Polynucleotide | ApoA1-IL2 fusion |
| 25 | Peptide | ApoA1-IL2 F42A fusion |
| 26 | Polynucleotide | ApoA1-IL2 F42A fusion |
| 27 | Peptide | ApoA1-IL2 F42A Y45A fusion |
| 28 | Polynucleotide | ApoA1-IL2 F42A Y45A fusion |
| 29 | Peptide | ApoA1-IL2 F42A Y45A L72G fusion |
| 30 | Polynucleotide | ApoA1-IL2 F42A Y45A L72G fusion |
| 31 | Peptide | ApoA1-IL2 F42A Y45A L72G C125A fusion |
| 32 | Polynucleotide | ApoA1-IL2 F42A Y45A L72G C125A fusion |
| 33 | Peptide | GFPnb-ApoA1-IL2 fusion |
| 34 | Polynucleotide | GFPnb-ApoA1-IL2 fusion |
| 35 | Peptide | ApoA1-IL4 fusion (human) |
| 36 | Polynucleotide | ApoA1-IL4 fusion (human) |
| 37 | Peptide | ApoA1-IL4 fusion (murine) |
| 38 | Polynucleotide | ApoA1-IL4 fusion (murine) |
| 39 | Peptide | ApoA1-IL4-CP fusion |
| 40 | Polynucleotide | ApoA 1-IL4-CP fusion |
| 41 | Peptide | ApoA1-IL38 fusion |
| 42 | Polynucleotide | ApoA1-IL38 fusion |
| 43 | Peptide | IL-4 (human) |
| 44 | Polynucleotide | IL-4 (human) |
| 45 | Peptide | IL-1β (human) |
| 46 | Polynucleotide | IL-1β (human) |
| 47 | Peptide | IL-2 (human) |
| 48 | Polynucleotide | IL-2 (human) |
| 49 | Peptide | IL-38 (human) |
| 50 | Polynucleotide | IL-38 (human) |
| 51 | Peptide | ApoA1 mimetic 18A |
| 52 | Peptide | ApoA1 mimetic 2F |
| 53 | Peptide | ApoA1 mimetic 37pA |
| 54 | Peptide | VHHCD8-apoA1 |
| 55 | Polynucleotide | VHHCD8-apoA1 |
| 56 | Peptide | VHHCD8-apoA1 cysteine mutant |
| 57 | Polynucleotide | VHHCD8-apoA1 cysteine mutant |
| 58 | Peptide | apoA1-IL2-HIS |
| 59 | Polynucleotide | apoA 1-IL2-HIS |
| 60 | Peptide | apoA1-IL2v4-HIS |
| 61 | Polynucleotide | apoA 1-IL2v4-HIS |
| 62 | Polynucleotide | ZNF UTR forward primer |
| 63 | Polynucleotide | ZNF UTR reverse primer |
| 64 | Polynucleotide | GAPDH forward primer |
| 65 | Polynucleotide | GAPDH reverse primer |
| 66 | Polynucleotide | TNF (1) forward primer |
| 67 | Polynucleotide | TNF (1) reverse primer |
| 68 | Polynucleotide | TNF (2) forward primer |
| 69 | Polynucleotide | TNF (2) reverse primer |
| 70 | Polynucleotide | TNF (3) forward primer |
| 71 | Polynucleotide | TNF (3) reverse primer |
| 72 | Polynucleotide | TNF (4) forward primer |
| 73 | Polynucleotide | TNF (4) reverse primer |
| 74 | Polynucleotide | TNF (5) forward primer |
| 75 | Polynucleotide | TNF (5) reverse primer |
| 76 | Polynucleotide | TNF (6) forward primer |
| 77 | Polynucleotide | TNF (6) reverse primer |
| 78 | Peptide | Human ApoA1 (without signal peptide) |
| 79 | Peptide | Thrombin cleavage site |
| 80 | Peptide | ApoA1-IL38 fusion |
| 81 | polynucleotide | ApoA1-IL38 fusion |
| 82 | Peptide | ApoA 1-IL-1β-HIS |
| 83 | polynucleotide | ApoA 1-IL-1β-HIS |
| 84 | Peptide | ApoA1-IL38-HIS |
| 85 | polynucleotide | ApoA1-IL38-HIS |

1. An apolipoprotein lipid nanoparticle comprising
a fusion protein comprising an apolipoprotein or an apolipoprotein mimetic and an immunomodulatory biomolecule; and
phospholipids;
wherein the immunomodulatory biomolecule is a protein that enhances or suppresses an immune response.

2. An apolipoprotein lipid nanoparticle comprising
a fusion protein comprising an apolipoprotein or an apolipoprotein mimetic and a rerouting molecule; and
phospholipids;
wherein the rerouting molecule is a molecule that allows that fusion protein to bind to a different target than to which the apolipoprotein or apolipoprotein mimetic would have bound and/or to bind to its intended target with a higher affinity.

3. An apolipoprotein lipid nanoparticle comprising
a fusion protein comprising an apolipoprotein or an apolipoprotein mimetic, an immunomodulatory biomolecule, and a rerouting molecule; and
phospholipids;
wherein the immunomodulatory biomolecule is a protein that enhances or suppresses an immune response; and
wherein the rerouting molecule is a molecule that allows that fusion protein to bind to a different target than to which the apolipoprotein or apolipoprotein mimetic would have bound and/or to bind to its intended target with a higher affinity.

4. An apolipoprotein lipid nanoparticle comprising
the fusion protein as defined in claim 1;
the fusion protein as defined in claim 2; and
phospholipids.

5. The apolipoprotein lipid nanoparticle according to any one of claims 1 to 4, wherein the apolipoprotein lipid nanoparticle further comprises sterols.

6. The apolipoprotein lipid nanoparticle according to any one of claims 1 to 5, wherein the apolipoprotein lipid nanoparticle further comprises lipids, preferably triglycerides.

7. The apolipoprotein lipid nanoparticle according to any one of claims 1 to 6, wherein said apolipoprotein lipid nanoparticle is a sphere, a ribbon or a disc.

8. The apolipoprotein lipid nanoparticle according to any one of claims 1 to 7, wherein at least a part of said fusion protein is exposed to the environment surrounding said apolipoprotein lipid nanoparticle, preferably wherein said immunomodulatory biomolecule and/or said rerouting molecule is exposed to the environment surrounding said apolipoprotein lipid nanoparticle.

9. The apolipoprotein lipid nanoparticle according to any one of claims 1 or 3 to 8, wherein the immunomodulatory biomolecule is selected from the group consisting of a cytokine, a chemokine, a hormone, a growth factor, a hematopoietic growth factor, and combinations thereof.

10. The apolipoprotein lipid nanoparticle according to claim 9, wherein the cytokine is selected from the group consisting of the IL-2 subfamily, the interferon subfamily, the IL-10 subfamily, the IL-1 family, the TGFbeta family, or the IL-17 family, and combinations thereof, more preferably wherein the cytokine is selected from the group consisting of IL-1p, IL-2, IL-4, IL-38, and combinations thereof; and/or
wherein the chemokine is selected from the group consisting of a CC chemokine, a CXC chemokine, a C chemokine, a CX₃C chemokine, and combinations thereof; and/or
wherein the growth factor is selected from the group consisting of VEGF, EGF, CNTF, LIF, Ephrins, FGF, GDNF, HDF, HDGF, IGF, KGF, MSF, NRG, BDNF, NGF, Neurotrophin, PGF, PDGF, RNLS, TCGF, TGF, TNF and WNT, and combinations thereof; and/or
wherein the hematopoietic growth factor is selected from the group consisting of IL-3, CSF-1 (M-CSF), GM-CSF, G-CSF, a member of the IL-12 family of interleukins or erythropoietin, and combinations thereof.

11. The apolipoprotein lipid nanoparticle according to claim 9 or 10, wherein the cytokine is IL-4.

12. The apolipoprotein lipid nanoparticle according to any one of claims 2 to 11, wherein the rerouting molecule is selected from an antibody or an antigen binding fragment thereof, a rerouting peptide or a rerouting protein, preferably wherein the rerouting peptide or rerouting protein is a ligand of a receptor present on the target.

13. The apolipoprotein lipid nanoparticle according to claim 12, wherein the antibody or antigen binding fragment thereof is selected from the group consisting of a Fab, a Fab₂, a scFv, a scFv-Fc, a dAb-Fc, a free light chain antibody, a half antibody, a bispecific Fab2, a Fab₃, a trispecific Fab3 a diabody, a bispecific diabody, a triabody, a trispecific triabody, a minibody, an IgG, an IgNAR, a monovalent IgG, a VₕH, and a variable new antigen receptor (VNAR).

14. The apolipoprotein lipid nanoparticle according to any one of claims 2 to 13, wherein the rerouting molecule is capable of binding to a hematopoietic stem and progenitor cell (HSPC), such as a hematopoietic stem cell (HSC), a multipotent progenitor (MPP), or a common myeloid progenitor cell (CMP).

15. The apolipoprotein lipid nanoparticle according to any one of claims 2 to 13, wherein the rerouting molecule is capable of binding to a myeloid cell selected from the group consisting of megakaryocyte, eosinophil, basophil, erythrocyte, monocyte such as dendritic cell or macrophage, and a neutrophil.

16. The apolipoprotein lipid nanoparticle according to claim 15, wherein the rerouting peptide is SIRPα.

17. The apolipoprotein lipid nanoparticle according to any one of claims 2 to 13, wherein the rerouting molecule is capable of binding to a non-myeloid cell, such as a non-myeloid immune cell or an endothelial cell.

18. The apolipoprotein lipid nanoparticle according to claim 17, wherein the rerouting molecule is capable of binding to lymphocytes, preferably T cells, more preferably CD8+ T cells.

19. The apolipoprotein lipid nanoparticle according to claim 17, wherein the rerouting molecule is an antibody or antigen binding fragment thereof specifically binding to CD8 or wherein the rerouting peptide is PD1, CD40L or GP120.

20. The apolipoprotein lipid nanoparticle according to any one of claims 1 to 19, wherein the apolipoprotein is an ApoA1, ApoA-1 Milano, ApoA4, ApoC3, ApoD, ApoE, ApoL1, ApoL3 or the apolipoprotein mimetic is a mimetic of an ApoA1, ApoA-1 Milano, ApoA4, ApoC3, ApoD, ApoE, ApoL1, ApoL3.

21. The apolipoprotein lipid nanoparticle according to any one of claims 1 to 20, wherein the apolipoprotein lipid nanoparticle comprises a payload, preferably wherein the payload is selected from a nucleic acid or a nucleic acid analog, a therapeutic, a biologic or combinations thereof.

22. Method of manufacturing an apolipoprotein lipid nanoparticle as defined in any one of claims 1 to 21, the method comprising the steps of:
a1) expressing and isolating one or more apolipoprotein fusion proteins to obtain one or more isolated apolipoprotein fusion proteins,
wherein the one or more apolipoprotein fusion proteins are selected from the group consisting of: an apolipoprotein or apolipoprotein mimetic fused to an immunomodulatory biomolecule;
an apolipoprotein or apolipoprotein mimetic fused to a rerouting molecule;
an apolipoprotein or apolipoprotein mimetic fused to an immunomodulatory biomolecule and a rerouting molecule; and combinations thereof; and/or
a2) chemically conjugating one or more apolipoproteins or apolipoprotein mimetics and isolating the one or more conjugated apolipoproteins to obtain one or more isolated conjugated apolipoproteins,
wherein the one or more conjugated apolipoproteins are selected from the group consisting of: an apolipoprotein or apolipoprotein mimetic conjugated to an immunomodulatory biomolecule;
an apolipoprotein or apolipoprotein mimetic conjugated to a rerouting molecule;
an apolipoprotein or apolipoprotein mimetic conjugated to an immunomodulatory biomolecule and a rerouting molecule; and combinations thereof; and
b) combining the one or more isolated apolipoprotein fusion proteins obtained in step a1 and/or the one or more isolated conjugated apolipoproteins obtained in step a2 with phospholipids, and optionally sterols and/or lipids, to obtain an apolipoprotein lipid nanoparticle.

23. An apolipoprotein lipid nanoparticle obtained by or obtainable by the method of claim 22.

24. A pharmaceutical composition comprising the apolipoprotein lipid nanoparticle according to any one of claims 1 to 21 or 23, and a pharmaceutically acceptable carrier.

25. The apolipoprotein lipid nanoparticle according to any one of claims 1 to 21 or 23 or the pharmaceutical composition according to claim 24 for use as a medicament.

26. The apolipoprotein lipid nanoparticle according to any one of claims 1 to 21 or 23 or the pharmaceutical composition according to claim 24 for use in the treatment of an immune related disorder.

27. The apolipoprotein lipid nanoparticle for use according to claim 26 or the pharmaceutical composition for use according to claim 26, wherein the immune related disorder is selected from the group consisting of cancer, inflammation, an infectious disease, an autoimmune disorder, allergy, organ transplant rejection, and graft-versus-host disease (GVH).

28. The apolipoprotein lipid nanoparticle for use according to claim 26 or the pharmaceutical composition for use according to claim 26, wherein the immunomodulatory biomolecule is IL-4 and wherein the immune related disorder is a state of hyperinflammation followed by immune paralysis, preferably wherein the hyperinflammation and/or the immune paralysis is caused by an infectious disease such as COVID-19, by sepsis, myocardial infarction, stroke, cancer, or multiple sclerosis.

29. The apolipoprotein lipid nanoparticle according to any one of claims 1, 3 to 21 or 23 or the pharmaceutical composition according to claim 24 for use in targeting said immunomodulatory biomolecule to a target cell.

30. The apolipoprotein lipid nanoparticle according to any one of claims 1, 3 to 13, 15, 16, 20, 21 or 23 or the pharmaceutical composition according to claim 24 for use in targeting said immunomodulatory biomolecule to a myeloid cell.

31. Use of the apolipoprotein lipid nanoparticle according to any one of claims 1, 3 to 21 or 23 for delivering an immunomodulatory biomolecule to a target, preferably wherein the target is a cell, tissue, and/or organ.

32. A fusion protein comprising an apolipoprotein or an apolipoprotein mimetic and an immunomodulatory biomolecule, wherein the immunomodulatory biomolecule is a protein that enhances or suppresses an immune response, for use in targeting said immunomodulatory biomolecule to a myeloid cell.

33. The fusion protein for use according to claim 32, wherein the fusion protein further comprises a rerouting molecule, wherein the rerouting molecule is a molecule that allows that fusion protein to bind to a different target than to which the apolipoprotein or apolipoprotein mimetic would have bound and/or to bind to its intended target with a higher affinity, preferably wherein the rerouting molecule is a rerouting molecule as defined in claim 15.

34. A fusion protein comprising an apolipoprotein or an apolipoprotein mimetic and a rerouting molecule, wherein the rerouting molecule is a molecule that allows that fusion protein to bind to a different target than to which the apolipoprotein or apolipoprotein mimetic would have bound and/or to bind to its intended target with a higher affinity.

35. The fusion protein according to claim 34, wherein the rerouting molecule is a rerouting molecule as defined in any one of claims 12 to 19.

36. The fusion protein according to claim 34 or 35, wherein the fusion protein further comprises an immunomodulatory biomolecule, wherein the immunomodulatory biomolecule is a protein that enhances or suppresses an immune response, preferably wherein the immunomodulatory biomolecule is an immunomodulatory biomolecule as defined in claim 9 or 10.

37. A nucleic acid encoding the fusion protein according to any one of claims 34 to 36.

38. A pharmaceutical composition comprising the fusion protein according to any one of claims 34 to 36 or the nucleic acid according to claim 37, and a pharmaceutically acceptable carrier.

39. The fusion protein according to any one of claims 34 to 36, the nucleic acid according to claim 37 or the pharmaceutical composition according to claim 38 for use as a medicament.

40. The fusion protein according to any one of claims 34 to 36, the nucleic acid according to claim 37 or the pharmaceutical composition according to claim 38 for use in the treatment of an immune related disorder, preferably wherein the immune related disorder is an immune related disorder selected from the group consisting of cancer, inflammation, an infectious disease, an autoimmune disorder, allergy, organ transplant rejection, and graft-versus-host disease (GVH).

41. The fusion protein according to claim 36, the nucleic acid encoding the fusion protein according to claim 36 or the pharmaceutical composition according to claim 38 when being dependent from claim 36 for use in targeting said immunomodulatory biomolecule to a target cell.

42. A fusion protein comprising an apolipoprotein or an apolipoprotein mimetic and interleukin-4 (IL-4).

43. The fusion protein according to claim 42, wherein the fusion protein further comprises a rerouting molecule, wherein the rerouting molecule is a molecule that allows that fusion protein to bind to a different target than to which the apolipoprotein or apolipoprotein mimetic would have bound and/or to bind to its intended target with a higher affinity, preferably wherein the rerouting molecule is a rerouting molecule as defined in any one of claims 12 to 19.

44. The fusion protein according to claim 42 or 43, wherein the apolipoprotein or apolipoprotein mimetic is as defined in claim 20.

45. A nucleic acid encoding the fusion protein according to any one of claims 42 to 44.

46. A pharmaceutical composition comprising the fusion protein according to any one of claims 42 to 44 or the nucleic acid according to claim 45, and a pharmaceutically acceptable carrier.

47. The fusion protein according to any one of claims 42 to 44, the nucleic acid according to claim 45, or the pharmaceutical composition according to claim 46 for use as a medicament.

48. The fusion protein according to any one of claims 42 to 44, the nucleic acid according to claim 45, or the pharmaceutical composition according to claim 46 for use in the treatment of an immune related disorder.

49. The fusion protein for use according to claim 48, the nucleic acid for use according to claim 48, or the pharmaceutical composition for use according to claim 48, wherein the immune related disorder is a state of hyperinflammation followed by immune paralysis, preferably wherein the hyperinflammation and/or the immune paralysis is caused by an infectious disease such as COVID-19, by sepsis, myocardial infarction, stroke, cancer, or multiple sclerosis.

50. The fusion protein according to any one of claims 42 to 44, the nucleic acid according to claim 45, or the pharmaceutical composition according to claim 46 for use in targeting IL-4 to a target cell.

51. The fusion protein according to any one of claims 42 to 44, the nucleic acid according to claim 45, or the pharmaceutical composition according to claim 46 for use in targeting IL-4 to a myeloid cell.

52. A fusion protein comprising a myeloid-targeting molecule and IL-4, wherein the myeloid-targeting molecule is capable of targeting the IL-4 to a myeloid cell.

53. The fusion protein according to claim 52, wherein the IL-4 is a polypeptide comprising an amino acid sequence at least 90%, 91%, 92%, 93%, 94%, 95%, 96%, 97%, 98%, 99% or 100% identical to SEQ ID NO. 43, or a circular permutation thereof.

54. The fusion protein according to claim 52 or 53, wherein the myeloid-targeting molecule is selected from an antibody or an antigen binding fragment thereof, a myeloid-targeting peptide or a myeloid-targeting protein, preferably wherein the myeloid-targeting peptide or myeloid-targeting protein is a ligand of a receptor present on the target.

55. The fusion protein according to claim 54, wherein the antibody or antigen binding fragment thereof is selected from a Fab, a Fab₂, a scFv, a scFv-Fc, a dAb-Fc, a free light chain antibody, a half antibody, a bispecific Fab2, a Fab₃, a trispecific Fab3 a diabody, a bispecific diabody, a triabody, a trispecific triabody, a minibody, an IgG, an IgNAR, a monovalent IgG, a VₕH or a VNAR.

56. A nucleic acid encoding the fusion protein according to any one of claims 52 to 55.

57. A nucleic acid comprising a nucleic acid sequence at least 70%, 75%, 80%, 85%, 90%, 91%, 92%, 93%, 94%, 95%, 96%, 97%, 98%, 99% or 100% identical to SEQ ID NO. 44 or comprising a nucleic acid sequence encoding a polypeptide having a sequence at least 90%, 91%, 92%, 93%, 94%, 95%, 96%, 97%, 98%, 99% or 100% identical to SEQ ID NO. 43 and further comprising means for targeted expression in a myeloid cell, wherein said mean are selected from:
a promoter for selective or inducible expression in said myeloid cell operatively linked to said nucleic acid; or
a viral expression vector comprising said nucleic acid capable of stably expressing said nucleic acid in said myeloid cell; or
a lipid nanoparticle comprising one or more apolipoproteins, phospholipids, said nucleic acid, and optionally sterol.

58. A pharmaceutical composition comprising the fusion protein according to any one of claims 52 to 55 or the nucleic acid according to claim 56 or 57, and a pharmaceutically acceptable carrier.

59. The fusion protein according to any one of claims 52 to 55, the nucleic acid according to claim 56 or 57, or the pharmaceutical composition according to claim 58 for use as a medicament.

60. The fusion protein according to any one of claims 52 to 55, the nucleic acid according to claim 56 or 57, or the pharmaceutical composition according to claim 58 for use in the treatment of an immune related disorder.

61. The fusion protein for use according to claim 60, the nucleic acid for use according to claim 60, or the pharmaceutical composition for use according to claim 60, wherein the immune related disorder is a state of hyperinflammation followed by immune paralysis, preferably wherein the hyperinflammation and/or the immune paralysis is caused by an infectious disease such as COVID-19, by sepsis, myocardial infarction or stroke.

62. In vivo, in vitro or ex vivo use of IL-4 in stimulating or promoting trained immunity in a cell, organ, tissue or an organism.


SEQUENCE LISTING (85 sequences)

SEQ ID NO: 1 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - DNA, 804 bp, Homo sapiens
  source 1..804: mol_type = unassigned DNA
atgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtga

SEQ ID NO: 3 - protein, 251 aa, synthetic construct
GLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 4 - DNA, 756 bp, synthetic construct
  source 1..756: mol_type = other DNA
ggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagtga

SEQ ID NO: 5 - protein, 257 aa, synthetic construct
HHHHHHGLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 6 - DNA, 774 bp, synthetic construct
  source 1..774: mol_type = other DNA
catcaccatcaccatcacggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagtga

SEQ ID NO: 7 - protein, 251 aa, synthetic construct
GLVPRGCIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 8 - DNA, 756 bp, synthetic construct
  source 1..756: mol_type = other DNA
ggcctggtgccgcgcggctgcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagtga

SEQ ID NO: 9 - protein, 251 aa, synthetic construct
GLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLCPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 10 - DNA, 756 bp, synthetic construct
  source 1..756: mol_type = other DNA
ggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgtgcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagtga

SEQ ID NO: 11 - protein, 251 aa, synthetic construct
GLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLCALEEYTKKLNTQ

SEQ ID NO: 12 - DNA, 756 bp, synthetic construct
  source 1..756: mol_type = other DNA
ggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgtgcgctctggaagagtacactaagaagcttaacacccagtga

SEQ ID NO: 13 - protein, 313 aa, Homo sapiens
GLTGSSTRLVPRGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 14 - DNA, 942 bp, Homo sapiens
  source 1..942: mol_type = unassigned DNA
ggcctgaccggttcttcaacgcgtctggtgccgcgcggcagcaaagtcgagcaagccgtagaaaccgagcccgagcctgagttgcgtcagcaaaccgaatggcagagcggtcaacgctgggaactggcattgggccgcttctgggattatctgcgctgggtgcagaccttatcagagcaagtacaagaggagttattgtcgtcgcaagtgacgcaggagcttcgtgccttaatggacgagacgatgaaagaactgaaggcatacaaatcagagcttgaggaacaattaacccccgtggccgaggagacacgcgcgcgtttatcaaaggagttgcaggctgctcaggctcgtttgggagctgacatggaagatgttcgcggtcgtcttgttcagtaccgtggcgaagtccaggctatgctgggccagtcaacggaggaacttcgcgtccgccttgcctcgcatttacgcaagttgcgtaaacgtcttttgcgcgacgctgatgaccttcaaaagcgtttggcagtataccaggctggtgcgcgtgaaggcgctgaacgtggtttgtctgcaatccgtgagcgtttgggtccactggtcgagcaaggccgcgtacgcgcggctactgttgggtcacttgcggggcaacctttgcaagagcgtgcccaagcgtggggggaacgtcttcgtgcccgcatggaagaaatgggaagccgcacgcgcgatcgtctggacgaagtgaaagaacaagttgcagaggttcgcgcaaaactggaagagcaggcccaacaaattcgtttgcaagctgaggcgtttcaggcgcgcttgaagtcatggttcgagccattggtagaggacatgcagcgtcaatgggcagggttagtggagaaagtgcaggcggcagttggtacttctgcttggccagtgccatccgacaatcattaa

SEQ ID NO: 15 - protein, 319 aa, synthetic construct
HHHHHHGLTGSSTRLVPRGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 16 - DNA, 960 bp, synthetic construct
  source 1..960: mol_type = other DNA
catcaccatcaccatcacggcctgaccggttcttcaacgcgtctggtgccgcgcggcagcaaagtcgagcaagccgtagaaaccgagcccgagcctgagttgcgtcagcaaaccgaatggcagagcggtcaacgctgggaactggcattgggccgcttctgggattatctgcgctgggtgcagaccttatcagagcaagtacaagaggagttattgtcgtcgcaagtgacgcaggagcttcgtgccttaatggacgagacgatgaaagaactgaaggcatacaaatcagagcttgaggaacaattaacccccgtggccgaggagacacgcgcgcgtttatcaaaggagttgcaggctgctcaggctcgtttgggagctgacatggaagatgttcgcggtcgtcttgttcagtaccgtggcgaagtccaggctatgctgggccagtcaacggaggaacttcgcgtccgccttgcctcgcatttacgcaagttgcgtaaacgtcttttgcgcgacgctgatgaccttcaaaagcgtttggcagtataccaggctggtgcgcgtgaaggcgctgaacgtggtttgtctgcaatccgtgagcgtttgggtccactggtcgagcaaggccgcgtacgcgcggctactgttgggtcacttgcggggcaacctttgcaagagcgtgcccaagcgtggggggaacgtcttcgtgcccgcatggaagaaatgggaagccgcacgcgcgatcgtctggacgaagtgaaagaacaagttgcagaggttcgcgcaaaactggaagagcaggcccaacaaattcgtttgcaagctgaggcgtttcaggcgcgcttgaagtcatggttcgagccattggtagaggacatgcagcgtcaatgggcagggttagtggagaaagtgcaggcggcagttggtacttctgcttggccagtgccatccgacaatcattaa

SEQ ID NO: 17 - protein, 313 aa, synthetic construct
GLTGCSTRLVPRGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQACVGTSAAPVPSDNH

SEQ ID NO: 18 - DNA, 942 bp, synthetic construct
  source 1..942: mol_type = other DNA
ggcctgaccggttgttcaacgcgtctggtgccgcgcggcagcaaagtcgagcaagccgtagaaaccgagcccgagcctgagttgcgtcagcaaaccgaatggcagagcggtcaacgctgggaactggcattgggccgcttctgggattatctgcgctgggtgcagaccttatcagagcaagtacaagaggagttattgtcgtcgcaagtgacgcaggagcttcgtgccttaatggacgagacgatgaaagaactgaaggcatacaaatcagagcttgaggaacaattaacccccgtggccgaggagacacgcgcgcgtttatcaaaggagttgcaggctgctcaggctcgtttgggagctgacatggaagatgttcgcggtcgtcttgttcagtaccgtggcgaagtccaggctatgctgggccagtcaacggaggaacttcgcgtccgccttgcctcgcatttacgcaagttgcgtaaacgtcttttgcgcgacgctgatgaccttcaaaagcgtttggcagtataccaggctggtgcgcgtgaaggcgctgaacgtggtttgtctgcaatccgtgagcgtttgggtccactggtcgagcaaggccgcgtacgcgcggctactgttgggtcacttgcggggcaacctttgcaagagcgtgcccaagcgtggggggaacgtcttcgtgcccgcatggaagaaatgggaagccgcacgcgcgatcgtctggacgaagtgaaagaacaagttgcagaggttcgcgcaaaactggaagagcaggcccaacaaattcgtttgcaagctgaggcgtttcaggcgcgcttgaagtcatggttcgagccattggtagaggacatgcagcgtcaatgggcagggttagtggagaaagtgcaggcggcagttggtacttctgcttgcccagtgccatccgacaatcattaa

SEQ ID NO: 19 - protein, 313 aa, synthetic construct
GLTGSSTRLVPRGSKVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQACVGTSAAPVPSDNH

SEQ ID NO: 20 - DNA, 942 bp, synthetic construct
  source 1..942: mol_type = other DNA
ggcctgaccggttcttcaacgcgtctggtgccgcgcggcagcaaagtcgagcaagccgtagaaaccgagcccgagcctgagttgcgtcagcaaaccgaatggcagagcggtcaacgctgggaactggcattgggccgcttctgggattatctgcgctgggtgcagaccttatcagagcaagtacaagaggagttattgtcgtcgcaagtgacgcaggagcttcgtgccttaatggacgagacgatgaaagaactgaaggcatacaaatcagagcttgaggaacaattaacccccgtggccgaggagacacgcgcgcgtttatcaaaggagttgcaggctgctcaggctcgtttgggagctgacatggaagatgttcgcggtcgtcttgttcagtaccgtggcgaagtccaggctatgctgggccagtcaacggaggaacttcgcgtccgccttgcctcgcatttacgcaagttgcgtaaacgtcttttgcgcgacgctgatgaccttcaaaagcgtttggcagtataccaggctggtgcgcgtgaaggcgctgaacgtggtttgtctgcaatccgtgagcgtttgggtccactggtcgagcaaggccgcgtacgcgcggctactgttgggtcacttgcggggcaacctttgcaagagcgtgcccaagcgtggggggaacgtcttcgtgcccgcatggaagaaatgggaagccgcacgcgcgatcgtctggacgaagtgaaagaacaagttgcagaggttcgcgcaaaactggaagagcaggcccaacaaattcgtttgcaagctgaggcgtttcaggcgcgcttgaagtcatggttcgagccattggtagaggacatgcagcgtcaatgggcagggttagtggagaaagtgcaggcggcagttggtacttctgcttgcccagtgccatccgacaatcattaa

SEQ ID NO: 21 - protein, 437 aa, synthetic construct
GLTGSSTRDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSSGSGSGSGSGSGASWSHPQFEK

SEQ ID NO: 22 - DNA, 1314 bp, synthetic construct
  source 1..1314: mol_type = other DNA
ggcctgaccggttcttcaacgcgtgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcaccagtgcgctccttgaactgtactctccgcgactcgcagcagaagtccttggtgatgagcgggccatacgagttgaaggcgttacatttacaggggcaggacatggagcaacaggtggtattctctatgtcgtttgtgcagggcgaagaaagcaatgataagatccctgtggctttgggtttaaaagagaaaaatctctatctctcatgtgtactcaaggatgataaacctactttacagttagaaagcgtagatcctaagaattacccaaagaaaaaaatggaaaagcggtttgtgtttaataaaattgagattaataataaattagagtttgaaagtgcccaattccctaactggtatatttccaccagtcaggctgagaatatgccggtgttcttaggcggcaccaagggtggtcaggacattacagactttaccatgcaattcgtatcatcgggctctggtagcggctctggttctggttctggtgctagctggagccatccgcagtttgagaaataa

SEQ ID NO: 23 - protein, 417 aa, synthetic construct
GLTGSSTRDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSGSGSGSGSGASWSHPQFEK

SEQ ID NO: 24 - DNA, 1254 bp, synthetic construct
  source 1..1254: mol_type = other DNA
ggcctgaccggttcttcaacgcgtgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcgccgaccagcagcagcaccaagaagacccagctgcaactggaacacctgctgctggacctgcaaatgattctgaacggtatcaacaactacaagaacccgaaactgacccgtatgctgaccttcaagttttatatgccgaagaaagcgaccgagctgaagcacctgcaatgcctggaggaagagctgaaaccgctggaagaggtgctgaacctggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcaccttttgccagagcatcattagcaccctgaccggctctggtagcggctctggttctggttctggtgctagctggagccatccgcagtttgagaaataa

SEQ ID NO: 25 - protein, 417 aa, synthetic construct
GLTGSSTRDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGSGSGSGSGSGASWSHPQFEK

SEQ ID NO: 26 - DNA, 1254 bp, synthetic construct
  source 1..1254: mol_type = other DNA
ggcctgaccggttcttcaacgcgtgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcgccgaccagcagcagcaccaagaagacccagctgcaactggaacacctgctgctggacctgcaaatgattctgaacggtatcaacaactacaagaacccgaaactgacccgtatgctgaccgcgaagttttatatgccgaagaaagcgaccgagctgaagcacctgcaatgcctggaggaagagctgaaaccgctggaagaggtgctgaacctggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcaccttttgccagagcatcattagcaccctgaccggctctggtagcggctctggttctggttctggtgctagctggagccatccgcagtttgagaaataa

SEQ ID NO: 27 - protein, 417 aa, synthetic construct
GLTGSSTRDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTAKFAMPKKATELKHLQCLEEELKPLEEVLNLAQSKNFHLRPRDLISNINVIVLELKGSETTFMCEYADETATIVEFLNRWITFCQSIISTLTGSGSGSGSGSGASWSHPQFEK

SEQ ID NO: 28 - DNA, 1254 bp, synthetic construct
  source 1..1254: mol_type = other DNA
ggcctgaccggttcttcaacgcgtgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcgccgaccagcagcagcaccaagaagacccagctgcaactggaacacctgctgctggacctgcaaatgattctgaacggtatcaacaactacaagaacccgaaactgacccgtatgctgaccgcgaagtttgctatgccgaagaaagcgaccgagctgaagcacctgcaatgcctggaggaagagctgaaaccgctggaagaggtgctgaacctggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcaccttttgccagagcatcattagcaccctgaccggctctggtagcggctctggttctggttctggtgctagctggagccatccgcagtttgagaaataa

SEQ ID NO: 29 - protein, 417 aa, synthetic construct
GLTGSSTRDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFCQSIISTLTGSGSGSGSGSGASWSHPQFEK

SEQ ID NO: 30 - DNA, 1254 bp, synthetic construct
  source 1..1254: mol_type = other DNA
ggcctgaccggttcttcaacgcgtgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcgccgaccagcagcagcaccaagaagacccagctgcaactggaacacctgctgctggacctgcaaatgattctgaacggtatcaacaactacaagaacccgaaactgacccgtatgctgaccgcgaagtttgctatgccgaagaaagcgaccgagctgaagcacctgcaatgcctggaggaagagctgaaaccgctggaagaggtgctgaacggggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcaccttttgccagagcatcattagcaccctgaccggctctggtagcggctctggttctggttctggtgctagctggagccatccgcagtttgagaaataa

SEQ ID NO: 31 - protein, 417 aa, synthetic construct
GLTGSSTRDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGSGSGSGSGSGASWSHPQFEK

SEQ ID NO: 32 - DNA, 1272 bp, synthetic construct
  source 1..1272: mol_type = other DNA
catcaccatcaccatcacggcctgaccggttcttcaacgcgtgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcgccgaccagcagcagcaccaagaagacccagctgcaactggaacacctgctgctggacctgcaaatgattctgaacggtatcaacaactacaagaacccgaaactgacccgtatgctgaccgcgaagtttgctatgccgaagaaagcgaccgagctgaagcacctgcaatgcctggaggaagagctgaaaccgctggaagaggtgctgaacggggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcacctttgcccagagcatcattagcaccctgaccggctctggtagcggctctggttctggttctggtgctagctggagccatccgcagtttgagaaataa

SEQ ID NO: 33 - protein, 525 aa, synthetic construct
GLVPRGSQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKASGSGSGSGSGSGDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLCPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTTR

SEQ ID NO: 34 - DNA, 1578 bp, synthetic construct
  source 1..1578: mol_type = other DNA
ggcctggtgccgcgcggcagccaggttcaactggtggaaagcggcggtgctctggtacaaccgggcggtagtctgcgcctgagctgtgccgcaagcggtttcccagtcaaccgctactctatgcgttggtatcgccaggcgcctggtaaagaacgtgaatgggttgccggcatgagcagtgcgggcgatcgttctagttacgaggactctgttaaaggtcgttttacaattagccgtgatgatgcgcgcaataccgtgtatctgcaaatgaacagtctgaagccggaggacaccgcagtatattattgcaatgtcaacgtggggtttgaatattggggccaggggactcaggtgacggtgagctctaaagctagcggctcgggtagcggttctggttcgggtagcggtgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgtgcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttcaggtagtggttcaggcagtggttcaggtaccgcgcctacttcatcaagcaccaaaaaaactcaattacaattagaacatttattgttagatctgcaaatgattctgaacggtatcaacaactacaagaacccgaaactgacccgtatgctgaccttcaagttttatatgccgaagaaagcgaccgagctgaagcacctgcaatgcctggaggaagagctgaaaccgctggaagaggtgctgaacctggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcaccttttgccagagcatcattagcaccctgaccacgcgttaa

SEQ ID NO: 35 - protein, 419 aa, synthetic construct
HHHHHHGLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTASHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSASGGSGGSGGSWSHPQFEK

SEQ ID NO: 36 - DNA, 1260 bp, synthetic construct
  source 1..1260: mol_type = other DNA
catcaccatcaccatcacggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgctagccataaatgtgatattaccctgcaggaaattattaaaaccctgaatagcctgaccgaacagaaaaccctgtgtaccgaactgaccgttaccgatatttttgcagcaagcaaaaataccaccgaaaaagaaaccttttgtcgtgcagcaaccgttctgcgtcagttttatagccatcatgaaaaagatacccgttgtctgggtgcaaccgcacagcagtttcatcgtcataaacagctgattcgttttctgaaacgtctggatcgtaatctgtggggtctggcaggtctgaatagctgtccggttaaagaagcaaatcagagcaccctggaaaattttctggaacgtctgaaaaccattatgcgtgaaaaatatagcaaatgtagcagcgctagcggtggttctggtggttctggtggttcttggagccatccgcagtttgagaaataa

SEQ ID NO: 37 - protein, 410 aa, synthetic construct
HHHHHHGLVPRGSIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTASHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSASGGSGGSGGSWSHPQFEK

SEQ ID NO: 38 - DNA, 1233 bp, synthetic construct
  source 1..1233: mol_type = other DNA
catcaccatcaccatcacggcctggtgccgcgcggcagcatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgctagccacatccatggctgcgacaagaaccacctgcgtgaaatcattggcatcctgaacgaagtgaccggcgagggcaccccgtgcaccgagatggacgtgccgaacgttctgaccgcgaccaagaacaccaccgagagcgaactggtgtgccgtgcgagcaaagttctgcgtatcttctacctgaagcacggtaaaaccccgtgcctgaagaaaaacagcagcgtgctgatggagctgcagcgtctgttccgtgcgtttcgttgcctggatagcagcattagctgcaccatgaacgaaagcaagagcaccagcctgaaagacttcctggagagcctgaaaagcattatgcagatggattacagcgctagcggtggttctggtggttctggtggttcttggagccatccgcagtttgagaaataa

SEQ ID NO: 39 - protein, 407 aa, synthetic construct
MIDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTASNTTEKETFCRAATVLRQFYSHHEKDTRCLGATAQQFHRHKQLIRFLKRLDRNLWGLAGLNSCPVKEANQSTLENFLERLKTIMREKYSKCSSGGNGGHKCDITLQEIIKTLNSLTEQKTLCTELTVTDIFAASKASLVPRGSHHHHHH

SEQ ID NO: 40 - DNA, 1224 bp, synthetic construct
  source 1..1224: mol_type = other DNA
atgatcgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgctagcaataccaccgaaaaagaaaccttttgtcgtgcagcaaccgttctgcgtcagttttatagccatcatgaaaaagatacccgttgtctgggtgcaaccgcacagcagtttcatcgtcataaacagctgattcgttttctgaaacgtctggatcgtaatctgtggggtctggcaggtctgaatagctgtccggttaaagaagcaaatcagagcaccctggaaaattttctggaacgtctgaaaaccattatgcgtgaaaaatatagcaaatgtagcagcggtggcaatggtggccataaatgtgatattaccctgcaggaaattattaaaaccctgaatagcctgaccgaacagaaaaccctgtgtaccgaactgaccgttaccgatatttttgcagcaagcaaagctagcctggtgccgcgcggcagccatcaccatcaccatcactaa

SEQ ID NO: 41 - protein, 437 aa, synthetic construct; identical to SEQ ID NO: 21

SEQ ID NO: 42 - DNA, 1314 bp, synthetic construct; identical to SEQ ID NO: 22
  source 1..1314: mol_type = other DNA

SEQ ID NO: 43 - protein, 153 aa, Homo sapiens [sequence omitted]

SEQ ID NO: 44 - DNA, 462 bp, Homo sapiens
  source 1..462: mol_type = unassigned DNA
atgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctga

SEQ ID NO: 45 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 46 - DNA, 809 bp, Homo sapiens
  source 1..809: mol_type = unassigned DNA
atggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttccta

SEQ ID NO: 47 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 48 - DNA, 462 bp, Homo sapiens
  source 1..462: mol_type = unassigned DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 49 - protein, 152 aa, Homo sapiens
MCSLPMARYYIIKYADQKALYTRDGQLLVGDPVADNCCAEKICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSW

SEQ ID NO: 50 - DNA, 459 bp, Homo sapiens
  source 1..459: mol_type = unassigned DNA
atgtgttccctccccatggcaagatactacataattaaatatgcagaccagaaggctctatacacaagagatggccagctgctggtgggagatcctgttgcagacaactgctgtgcagagaagatctgcatacttcctaacagaggcttggcccgcaccaaggtccccattttcctggggatccagggagggagccgctgcctggcatgtgtggagacagaagaggggccttccctacagctggaggatgtgaacattgaggaactgtacaaaggtggtgaagaggccacacgcttcaccttcttccagagcagctcaggctccgccttcaggcttgaggctgctgcctggcctggctggttcctgtgtggcccggcagagccccagcagccagtacagctcaccaaggagagtgagccctcagcccgtaccaagttttactttgaacagagctggtag

SEQ ID NO: 51 - protein, 18 aa, synthetic construct
DWLKAFYDKVAEKLKEAF

SEQ ID NO: 52 - protein, 18 aa, synthetic construct
FAEKLKEAVKDYFAKLWD

SEQ ID NO: 53 - protein, 37 aa, synthetic construct
DWLKAFYDKVAEKLKEAFPDWLKAFYDKVAEKLKEAF

SEQ ID NO: 54 - protein, 389 aa, synthetic construct
MTGQVQLQESGGGLVQAGGSLRLSCAASGFTFDDYAIGWFRQAPGKEREGVSCINNSDGSGYVADSVKGRFTISSDNAKNTVYLQMNSLKPEDTAVYYCAAAGDGGCYYGLDYWGKGTQVTVSSGGLEVTRSGSGSGSDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGDHHHHHH

SEQ ID NO: 55 - DNA, 1170 bp, synthetic construct
  source 1..1170: mol_type = other DNA
atgaccggtcaggttcagttacaggagtccggtgggggactggtgcaggccgggggttctctgcgccttagctgcgctgcctccggtttcaccttcgatgattacgctattggctggttccgtcaggctcctggcaaggagcgtgaaggagtatcctgtatcaacaattcggacgggagtgggtatgtagcggacagtgttaaaggtcgctttacgatttcctccgacaacgccaagaacaccgtctaccttcaaatgaactccctgaagccagaagataccgctgtttattactgtgccgcagcgggtgacggtggttgttactacggcttggactactggggtaaaggaacccaggttactgtctcatccgggggactggaagtgacgcgttcagggtcaggtagtggatctgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggtgatcatcatcatcatcatcattaa

SEQ ID NO: 56 - protein, 389 aa, synthetic construct
MTGQVQLQESGGGLVQAGGSLRLSCAASGFTFDDYAIGWFRQAPGKEREGVSCINNSDGSGYVADSVKGRFTISSDNAKNTVYLQMNSLKPEDTAVYYCAAAGDGGCYYGLDYWGKGTQVTVSSGGLECKLSGSGSGSDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGDHHHHHH

SEQ ID NO: 57 - DNA, 1170 bp, synthetic construct
  source 1..1170: mol_type = other DNA
atgaccggtcaggttcagttacaggagtccggtgggggactggtgcaggccgggggttctctgcgccttagctgcgctgcctccggtttcaccttcgatgattacgctattggctggttccgtcaggctcctggcaaggagcgtgaaggagtatcctgtatcaacaattcggacgggagtgggtatgtagcggacagtgttaaaggtcgctttacgatttcctccgacaacgccaagaacaccgtctaccttcaaatgaactccctgaagccagaagataccgctgtttattactgtgccgcagcgggtgacggtggttgttactacggcttggactactggggtaaaggaacccaggttactgtctcatccgggggactggaatgcaagctttcagggtcaggtagtggatctgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggtgatcatcatcatcatcatcattaa

SEQ ID NO: 58 - protein, 408 aa, synthetic construct
MDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSGSGSGSGSGASHHHHHH

SEQ ID NO: 59 - DNA, 1227 bp, synthetic construct
  source 1..1227: mol_type = other DNA
atggatgatccacctcaaagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggaaatggagttatatcgccaaaaggtcgagccgctgcgcgcagagctgcaggagggcgcgcgccagaagctgcacgagctgcaagagaagctgagcccattaggtgaagaaatgcgtgaccgcgcgcgcgcacatgtcgacgcattacgcacacatttagcgccgtactctgacgagctgcgccagcgcttagcggcgcgccttgaggctctcaaggagaacggtggagctcgcttagcagaatatcacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcgccgaccagcagcagcaccaagaagacccagctgcaactggaacacctgctgctggacctgcaaatgattctgaacggtatcaacaactacaagaacccgaaactgacccgtatgctgaccttcaagttttatatgccgaagaaagcgaccgagctgaagcacctgcaatgcctggaggaagagctgaaaccgctggaagaggtgctgaacctggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcaccttttgccagagcatcattagcaccctgaccggctctggtagcggctctggttctggttctggtgctagccatcaccatcaccatcattaa

SEQ ID NO: 60 - protein, 408 aa, synthetic construct
MDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLTGSGSGSGSGSGASHHHHHH

SEQ ID NO: 61 - DNA, 1227 bp, synthetic construct
  source 1..1227: mol_type = other DNA
atggatgatccacctcaaagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggaaatggagttatatcgccaaaaggtcgagccgctgcgcgcagagctgcaggagggcgcgcgccagaagctgcacgagctgcaagagaagctgagcccattaggtgaagaaatgcgtgaccgcgcgcgcgcacatgtcgacgcattacgcacacatttagcgccgtactctgacgagctgcgccagcgcttagcggcgcgccttgaggctctcaaggagaacggtggagctcgcttagcagaatatcacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcgccgaccagcagcagcaccaagaagacccagctgcaactggaacacctgctgctggacctgcaaatgattctgaacggtatcaacaactacaagaacccgaaactgacccgtatgctgaccgcgaagtttgctatgccgaagaaagcgaccgagctgaagcacctgcaatgcctggaggaagagctgaaaccgctggaagaggtgctgaacggggcgcaaagcaagaacttccacctgcgtccgcgtgacctgatcagcaacattaacgtgatcgttctggaactgaaaggcagcgagaccacctttatgtgcgaatatgcggatgagaccgcgaccattgttgagttcctgaaccgttggatcacctttgcccagagcatcattagcaccctgaccggctctggtagcggctctggttctggttctggtgctagccatcaccatcaccatcattaa

SEQ ID NO: 62 - DNA, 20 bp, synthetic construct
  source 1..20: mol_type = other DNA
aagcactttgacaaccgtga

SEQ ID NO: 63 - DNA, 20 bp, synthetic construct
  source 1..20: mol_type = other DNA
ggaggaattttgtggagcaa

SEQ ID NO: 64 - DNA, 21 bp, synthetic construct
  source 1..21: mol_type = other DNA
ccccggtttctataaatgagc

SEQ ID NO: 65 - DNA, 20 bp, synthetic construct
  source 1..20: mol_type = other DNA
aagaagatgcggctgactgt

SEQ ID NO: 66 - DNA, 20 bp, synthetic construct
  source 1..20: mol_type = other DNA
agaggaccagctaagaggga

SEQ ID NO: 67 - DNA, 19 bp, synthetic construct
  source 1..19: mol_type = other DNA
agcttgtcaggggatgtgg

SEQ ID NO: 68 - DNA, 20 bp, synthetic construct
  source 1..20: mol_type = other DNA
caggcaggttctcttcctct

SEQ ID NO: 69 - DNA, 20 bp, synthetic construct
  source 1..20: mol_type = other DNA
gctttcagtgctcatggtgt

SEQ ID NO: 70 - DNA, 20 bp, synthetic construct
  source 1..20: mol_type = other DNA
gtgcttgttcctcagcctct

SEQ ID NO: 71 - DNA, 20 bp, synthetic construct
  source 1..20: mol_type = other DNA
atcactccaaagtgcagcag

SEQ ID NO: 72 - DNA, 20 bp, synthetic construct
  source 1..20: mol_type = other DNA
tgtctggcacacagaagaca

SEQ ID NO: 73 - DNA, 20 bp, synthetic construct
  source 1..20: mol_type = other DNA
ccctgaggtgtctggttttc

SEQ ID NO: 74 - DNA, 20 bp, synthetic construct
  source 1..20: mol_type = other DNA
agccagctgttcctccttta

SEQ ID NO: 75 - DNA, 20 bp, synthetic construct
  source 1..20: mol_type = other DNA
ttagagagaggtccctgggg

SEQ ID NO: 76 - DNA, 20 bp, synthetic construct
  source 1..20: mol_type = other DNA
tgatggtaggcagaacttgg

SEQ ID NO: 77 - DNA, 20 bp, synthetic construct
  source 1..20: mol_type = other DNA
actaaggcctgtgctgttcc

SEQ ID NO: 78 - protein, 249 aa, Homo sapiens
RHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 79 - protein, 10 aa, synthetic construct
GLVPRGSIDD

SEQ ID NO: 80 - protein, 442 aa, synthetic construct
MHHHHHHGLTGSSTRDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTCSLPMARYYIIKYADQKALYTRDGQLLVGDPVADNCCAEKICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSWGSGSGSGSGSGASWSHPQFEK

SEQ ID NO: 81 - DNA, 1329 bp, synthetic construct
  source 1..1329: mol_type = other DNA
atgcatcaccatcaccatcacggcctgaccggttcttcaacgcgtgatgatccgccgcagagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggagatggagctctaccgccagaaggtggagccgctgcgcgcagagctgcaggagggcgcgcgccagaagctgcacgagctgcaagagaagctgagcccactgggcgaggagatgcgcgaccgcgcgcgcgcccatgtcgacgcgctgcgcacgcatctggcgccgtacagcgacgagctgcgccagcgcttagcggcgcgccttgaggctctcaaggagaacggcggggcccgcctggccgagtaccacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtacctgtagtttgccaatggcgcgctactatattatcaaatacgccgatcagaaagccttatatacacgggacgggcagttgttagtaggtgacccagtcgcggataactgctgtgctgaaaagatttgcatcttaccaaatcgcgggctggcgcgcacgaaagtgcccatcttcctgggaatccagggcgggagtcggtgtcttgcgtgtgtcgagacagaagagggtccctcgttacagttggaagatgtaaatatcgaggaactgtataaggggggcgaagaggccactagatttacgttctttcaaagttcatcggggagtgcctttagacttgaggcagcggcctggccaggctggttcctttgcggccccgctgagcctcaacagcctgttcaattaacaaaggaatccgaaccatcagccagaacgaaattctattttgaacagtcatggggctctggtagcggctctggttctggttctggtgctagctggagccatccgcagtttgagaaataa

SEQ ID NO: 82 - protein, 428 aa, synthetic construct
MDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSSGSGSGSGSGSGASHHHHHH

SEQ ID NO: 83 - DNA, 1284 bp, synthetic construct
  source 1..1284: mol_type = other DNA
atggatgatccacctcaaagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggaaatggagttatatcgccaaaaggtcgagccgctgcgcgcagagctgcaggagggcgcgcgccagaagctgcacgagctgcaagagaagctgagcccattaggtgaagaaatgcgtgaccgcgcgcgcgcacatgtcgacgcattacgcacacatttagcgccgtactctgacgagctgcgccagcgcttagcggcgcgccttgaggctctcaaggagaacggtggagctcgcttagcagaatatcacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtaccgcaccagtgcgctccttgaactgtactctccgcgactcgcagcagaagtccttggtgatgagcgggccatacgagttgaaggcgttacatttacaggggcaggacatggagcaacaggtggtattctctatgtcgtttgtgcagggcgaagaaagcaatgataagatccctgtggctttgggtttaaaagagaaaaatctctatctctcatgtgtactcaaggatgataaacctactttacagttagaaagcgtagatcctaagaattacccaaagaaaaaaatggaaaagcggtttgtgtttaataaaattgagattaataataaattagagtttgaaagtgcccaattccctaactggtatatttccaccagtcaggctgagaatatgccggtgttcttaggcggcaccaagggtggtcaggacattacagactttaccatgcaattcgtatcatcgggctctggtagcggctctggttctggttctggtgctagccatcaccatcaccatcat

SEQ ID NO: 84 - protein, 421 aa, synthetic construct
MDDPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGSGSGSGSGSGTCSLPMARYYIIKYADQKALYTRDGQLLVGDPVADNCCAEKICILPNRGLARTKVPIFLGIQGGSRCLACVETEEGPSLQLEDVNIEELYKGGEEATRFTFFQSSSGSAFRLEAAAWPGWFLCGPAEPQQPVQLTKESEPSARTKFYFEQSWGSGSGSASHHHHHH

SEQ ID NO: 85 - DNA, 1263 bp, synthetic construct
  source 1..1263: mol_type = other DNA
atggatgatccacctcaaagtccatgggatcgcgtgaaggacctggccactgtgtacgtggatgtgctcaaagacagcggcagagactatgtgtctcagtttgaaggatccgccttgggcaaacaattgaaccttaagctgctggacaactgggacagcgtgacgtccaccttcagcaagctgcgcgaacagctcggccctgtgacccaggaattctgggataacctggaaaaggagacagagggcctgcgccaggagatgagcaaggatctggaggaggtgaaggccaaggtgcagccgtacctggacgacttccagaagaagtggcaggaggaaatggagttatatcgccaaaaggtcgagccgctgcgcgcagagctgcaggagggcgcgcgccagaagctgcacgagctgcaagagaagctgagcccattaggtgaagaaatgcgtgaccgcgcgcgcgcacatgtcgacgcattacgcacacatttagcgccgtactctgacgagctgcgccagcgcttagcggcgcgccttgaggctctcaaggagaacggtggagctcgcttagcagaatatcacgccaaggccaccgagcatctgagcacgctcagcgagaaggccaagccggcgctcgaggacctgcgccaaggcctgctgccggtgctggagagcttcaaggtcagcttcctgagcgctctggaagagtacactaagaagcttaacacccagggttctggtagcggctctggttctggttctggtacctgtagtttgccaatggcgcgctactatattatcaaatacgccgatcagaaagccttatatacacgggacgggcagttgttagtaggtgacccagtcgcggataactgctgtgctgaaaagatttgcatcttaccaaatcgcgggctggcgcgcacgaaagtgcccatcttcctgggaatccagggcgggagtcggtgtcttgcgtgtgtcgagacagaagagggtccctcgttacagttggaagatgtaaatatcgaggaactgtataaggggggcgaagaggccactagatttacgttctttcaaagttcatcggggagtgcctttagacttgaggcagcggcctggccaggctggttcctttgcggccccgctgagcctcaacagcctgttcaattaacaaaggaatccgaaccatcagccagaacgaaattctattttgaacagtcatggggctctggtagcggatctgctagccatcaccatcaccatcat